(19) United States
(12) Patent Application Publication
(10) Pub. No.: US 2026/0066042 A1
(43) Pub. Date: Mar. 5, 2026

(54) METHODS AND SYSTEMS FOR HIGH-THROUGHPUT MOLECULAR ANALYSIS

(71) Applicant: OPENTRONS LABWORKS INC., W Long Island City, NY (US)

(72) Inventors: Henry H. LEE, Brookline, NY (US); Michael J. HAMMERLING, Long Island City, NY (US); Jon LAURENT, New York, NY (US); Haiping HAO, New York, NY (US); Melissa HOPKINS, New Rochelle, NY (US); Cybill DEL CASTILLO, Astoria, NY (US); Pradeep BUGGA, Charlestown, MA (US); Shinyoung KANG, Cambridge, MA (US)

(21) Appl. No.: 18/730,349

(22) PCT Filed: Jan. 30, 2023
(86) PCT No.: PCT/US2023/061601

§ 371 Date: Jul. 19, 2024

Related U.S. Application Data
(60) Provisional application No. 63/305,124, filed on Jan. 31, 2022.

(51) Int. Cl. G16B 20/50; C12N 15/10; C12Q 1/6806; C12Q 1/6851; C12Q 1/6869; G16B 30/00

(52) U.S. Cl. CPC G16B 20/50; C12N 15/1065; C12N 15/1096; C12Q 1/6806; C12Q 1/6851; C12Q 1/6869; G16B 30/00

(57) ABSTRACT

Provided herein are methods and systems for high throughput sequencing of nucleic acids for the surveillance of pathogens in a population and efficient identification of new pathogen variants of concern.

### CROSS REFERENCE

This application claims the benefit of U.S. Provisional Application No. 63/305,124, filed on Jan. 31, 2022, which is incorporated herein by reference in its entirety.

### BACKGROUND

Rapid and accurate diagnostic methods and systems for detecting and monitoring pathogens are important for timely patient diagnosis and intervention for infectious diseases. Polymerase chain reaction (PCR)-based methods are currently the most well-developed molecular techniques with a wide range of clinical applications including specific or broad-spectrum pathogen detection, evaluation of emerging novel infections, surveillance, early detection of biothreat agents, and antimicrobial resistance profiling. However, the outbreak of severe acute respiratory syndrome coronavirus 2 (SARS-CoV-2) and ensuing global pandemic have placed an overwhelming burden on healthcare institutions across the world and have revealed a lack of infrastructure capable of scaling quickly to counter pandemic-scale public health crises. Despite the success of increased testing infrastructure, new variants of SARS-CoV-2 have continually emerged throughout the pandemic. Existing technologies do not possess sufficient power, responsiveness, or scalability to be track and identify new variants efficiently in large population centers.

### SUMMARY

Disclosed herein are systems and methods for efficient analysis of nucleic acid molecules from patient samples and scalable surveillance of pathogens within patient populations of various sizes and demographics. The speed of analysis of patient-derived samples can be critical to monitoring and responding to the presence of an infectious pathogen within a patient population. For example, rapid identification of individuals having an infectious pathogen at ports of entry (e.g., airports, water-based vessel terminals or docks, and/or land-based checkpoints) to a monitored patient population can prevent or reduce the opportunity for the pathogen to spread within the patient population. Rapid identification and/or characterization of novel pathogen (e.g., by reconstruction of the pathogen's genome via molecular analysis of patient samples) within a community can aid with efforts to mitigate the exposure of the community to the novel pathogen and can be crucial to preventing the novel pathogen from escaping the patient population. For example, when a pathogen (e.g., a novel pathogen or a new instance of a known pathogen) is detected within a community, it can be important to rapidly assess the extent to which the pathogen has been spread to other individuals within the community, both with respect to geographical spread of the pathogen within the community and with respect to spread of the pathogen among individuals in the community belonging to demographic groups of interest, such as demographic groups at risk of severe illness. To this end, the establishment of scalable biosurveillance systems and methods for monitoring patient populations (e.g., as described herein) presents tremendous value to governance agencies tasked with monitoring and effecting policy for public health and for commercial efforts to study and/or respond to pathogens (e.g., via the development of vaccines, antibiotics, or medical stratagem for combating the pathogens), for instance, in place of systems and methods developed for a specific segment of a patient population community or that are limited by the available resources in individual segments of the patient population community, which can be unevenly distributed, heterogeneous in efficacy, and/or fraught by inefficiencies in mobilization or integration with other segments of the community.

Furthermore, the incorporation of improvements to individual components or steps of such systems and methods, for example as described herein, can drastically affect the efficacy, speed, scalability, and, ultimately, the feasibility of such systems and methods, especially when such systems and methods are applied to large patient populations, such as major metropolitan communities.

In various aspects, a method for reconstructing a genome of a pathogen comprises: determining a Ct value for a plurality of RNA molecules isolated from a sample; transporting the plurality of RNA molecules at a temperature no greater than −20° C. from a first location to a second location; performing a RT-PCR protocol on RNA from a biological sample to generate a plurality of cDNA molecules; and sequencing the modified nucleic acid at the second location to determine a nucleotide sequence of all or a portion of the plurality of cDNA molecules. In some aspects, the RT-PCR protocol is a one-step RT-PCR protocol. In some aspects, the input volume of RNA for the one-step RT-PCR protocol is at least 5 microliters. In some aspects, the elongation temperature of the one-step RT-PCR protocol is 60.5 degrees Celsius. In some aspects, the elongation time of the one-step RT-PCR protocol is 3 minutes. In some aspects, performing the one-step RT-PCR protocol comprises the use of an RMv1 or RMv2 primer set. In some aspects, performing the one-step RT-PCR protocol comprises the use of a primer, the primer comprising a 5′ end modification. In some aspects, the primer is biotinylated at the 5′ end of the primer. In some aspects, the method further comprises tagmenting the plurality of cDNA molecules. In some aspects, each cDNA molecule of the plurality of cDNA molecules is tagmented with polyethylene glycol (PEG). In some aspects, the method further comprises performing hybrid capture on at least a portion of the tagmented cDNA molecules. In some aspects, the method further comprises performing hybrid capture on a portion of the tagmented cDNA molecules derived from RNA molecules with a Ct value greater than 27, as determined using an RT-qPCR assay. In some aspects, performing the one-step RT-PCR protocol comprises the use of an exonuclease with a processivity of at least 60 nucleotides per second. In some aspects, performing the one-step RT-PCR protocol comprises the use of Taq polymerase. In some aspects, performing the one-step RT-PCR protocol comprises the use of a longer tiling primer than an A400 primer. In some aspects, performing the one-step RT-PCR protocol comprises the use of an A1200 primer.

In various aspects, a system for a high-throughput nucleic acid sequencing and analysis, the system comprises: a first analysis module configured to determine a plurality of respective amplification cycle threshold (Ct) values for each of a first plurality of nucleic acid populations; a second analysis module configured to perform a one-step amplification protocol on a subset of the first plurality of nucleic acid populations; a third analysis module comprising a molecular sequencer configured to determine a first plurality of nucleic acid sequences corresponding to each population of the subset of the first plurality of nucleic acid populations; and a computer system configured to determine a rate of mutation in a pathogen based on the first plurality of nucleic acid sequences. In some aspects, the RT-PCR protocol is a one-step RT-PCR protocol. In some aspects, the system further comprises a processing device configured to prepare the subset of the first plurality of nucleic acid populations for analysis by the second analysis module based on the respective Ct values determined by the first analysis module. In some aspects, preparing the subset of the first plurality of nucleic acid populations for analysis comprises automatically transferring the subset to respective wells of a processing container based on the respective Ct values determined by the first analysis module. In some aspects, the processing device is configured to prepare a subset of a second plurality of nucleic acid populations for analysis by the second analysis module based on respective Ct values of the second plurality of nucleic acid populations determined by a fourth analysis module, wherein each population of the subset of the second plurality of nucleic acid populations has a Ct value less than a threshold Ct value. In some aspects, the third analysis module is configured to determine a second plurality of nucleic acid sequences corresponding to each population of the subset of the second plurality of nucleic acid populations. In some aspects, each of the first plurality of nucleic acid populations is derived from a respective subject of a first population of subjects, and wherein each of the second plurality of nucleic acid populations is derived from a respective subject of a second population of subjects, at least one of the first population of subjects and at least one of the second population of subjects having an unknown risk of exposure to a pathogen of interest. In some aspects, the subjects of the first population of subjects are located in a different geographical area than the subjects of the second population of subjects. In some aspects, the computer system is further configured to determine a rate of transmission of the pathogen based on the first plurality of nucleic acid sequences and the second plurality of nucleic acid sequences. In some aspects, the threshold Ct value is 37. In some aspects, the system further comprises a computer system configured to determine a rate of mutation in a pathogen based on the first plurality of nucleic acid sequences. In some aspects, the rate of mutation is based on the first plurality of nucleic acid sequences and the second plurality of nucleic acid sequences. In some aspects, the input volume of first plurality of nucleic acid molecules for the one-step amplification protocol is at least 5 microliters. In some aspects, the elongation temperature of the one-step amplification protocol is 60.5 degrees Celsius. In some aspects, the elongation time of the one-step amplification protocol is 3 minutes. In some aspects, performing the one-step amplification protocol comprises the use of an RMv1 or RMv2 primer set. In some aspects, performing the one-step amplification protocol comprises the use of a primer, the primer comprising a 5′ end modification. In some aspects, the primer is biotinylated at the 5′ end of the primer. In some aspects, the system is configured to tagment the plurality of nucleic acid molecules. In some aspects, each cDNA molecule of the plurality of nucleic acid molecules is tagmented with polyethylene glycol (PEG). In some aspects, the system is configured to perform hybrid capture on at least a portion of the tagmented nucleic acid molecules. In some aspects, performing the one-step amplification protocol comprises the use of an exonuclease with a processivity of at least 60 nucleotides per second. In some aspects, performing the one-step amplification protocol comprises the use of Taq polymerase. In some aspects, performing the one-step amplification protocol comprises the use of a longer tiling primer than an A400 primer. In some aspects, performing the one-step amplification protocol comprises the use of an A1200 primer.

In some aspects, provided herein is a method for reconstructing a genome of a pathogen, comprising: determining an amplification cycle threshold (Ct) value for a plurality of ribonucleic acid (RNA) molecules isolated from a biological sample; transporting the plurality of RNA molecules at a temperature no greater than −20° C. from a first location to a second location; performing a reverse transcription polymerase chain reaction (RT-PCR) protocol on at least a portion of the plurality of RNA molecules to generate a plurality of complementary deoxyribonucleic acid (cDNA) molecules; and sequencing all or a portion of the plurality of cDNA molecules at the second location to determine nucleotide sequences of the all or the portion of the plurality of cDNA molecules.

In some aspects, provided herein, is a system for high-throughput nucleic acid sequencing and analysis, the system comprising: a first analysis module configured to determine a plurality of respective amplification cycle threshold (Ct) values for each of a first plurality of nucleic acid populations; a second analysis module configured to perform a one-step amplification protocol on a subset of the first plurality of nucleic acid populations; a third analysis module comprising a molecular sequencer configured to determine a first plurality of nucleic acid sequences corresponding to each population of the subset of the first plurality of nucleic acid populations; and a computer system configured to determine a rate of mutation in a pathogen based at least in part on the first plurality of nucleic acid sequences.

In some aspect, provided herein is a system for high-throughput nucleic acid sequencing and analysis, the system comprising: a first analysis module configured to determine a plurality of respective amplification cycle threshold (Ct) values for each of a first plurality of nucleic acid populations; a second analysis module configured to select a subset of the first plurality of nucleic acid populations; a third analysis module configured to perform a one-step amplification protocol on the subset of the first plurality of nucleic acid populations; a fourth analysis module comprising a molecular sequencer configured to determine a first plurality of nucleic acid sequences corresponding to each population of the subset of the first plurality of nucleic acid populations; and a computer system configured to determine a mutation in a pathogen based at least in part on the first plurality of nucleic acid sequences.

### INCORPORATION BY REFERENCE

All publications, patents, and patent applications mentioned in this specification are herein incorporated by reference to the same extent as if each individual publication, patent, or patent application is specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a diagram illustrating systems for high-throughput nucleic acid molecule analysis, in accordance with embodiments.

FIG. 1B is a diagram illustrating additional configurations of systems for high-throughput nucleic acid molecule analysis, in accordance with embodiments.

FIG. 1C is a diagram illustrating further embodiments of systems for high-throughput nucleic acid molecule analysis, in accordance with embodiments.

FIG. 2A shows a comparison of RT-PCR Ct values for RNA samples analyzed at a first site and a second site using unmodified methods, in accordance with embodiments. FIG. 2B shows a comparison of RT-PCR Ct values for RNA samples analyzed at a first site and a second site using modified methods, in accordance with embodiments.

FIG. 3A shows an evaluation of a one-step RT-PCR protocol with respect to production of genomic cDNA for a positive sample, in accordance with embodiments. FIG. 3B shows an evaluation of a one-step RT-PCR protocol with respect to production of genomic cDNA for a positive sample, in accordance with embodiments. FIG. 3C shows an evaluation of a one-step RT-PCR protocol with respect to production of genomic cDNA for a positive sample, in accordance with embodiments.

FIG. 4A shows an electrophoresis gel illustrating differences between one-step and two-step protocols, in accordance with embodiments. FIG. 4B shows a comparison of one-step and two-step protocols with respect to uncalled bases, in accordance with embodiments. FIG. 4C shows a comparison of one-step and two-step protocols with respect to average genome coverage data, in accordance with embodiments. FIG. 4D shows a comparison of one-step and two-step protocols with respect to genome length data, in accordance with embodiments.

FIG. 5A shows an analysis of nucleic acid analysis protocols on uncalled bases, in accordance with embodiments. FIG. 5B shows an analysis of nucleic acid analysis protocols on uncalled bases, in accordance with embodiments. FIG. 5C shows an analysis of nucleic acid analysis protocols on uncalled bases, in accordance with embodiments. FIG. 5D shows an analysis of nucleic acid analysis protocols on uncalled bases, in accordance with embodiments.

FIG. 6A shows an analysis of nucleic acid analysis protocols on average genome coverage, in accordance with embodiments. FIG. 6B shows an analysis of nucleic acid analysis protocols on average genome coverage, in accordance with embodiments. FIG. 6C shows an analysis of nucleic acid analysis protocols on average genome coverage, in accordance with embodiments. FIG. 6D shows an analysis of nucleic acid analysis protocols on average genome coverage, in accordance with embodiments.

FIG. 7A shows an analysis of nucleic acid analysis protocols on genome length, in accordance with embodiments. FIG. 7B shows an analysis of nucleic acid analysis protocols on genome length, in accordance with embodiments. FIG. 7C shows an analysis of nucleic acid analysis protocols on genome length, in accordance with embodiments. FIG. 7D shows an evaluation of nucleic acid analysis protocols on genome length, in accordance with embodiments.

FIG. 8A shows an evaluation of a one-step RT-PCR master mix, in accordance with embodiments. FIG. 8B shows an evaluation of a one-step RT-PCR master mix, in accordance with embodiments. FIG. 8C shows an evaluation of a one-step RT-PCR master mix, in accordance with embodiments. FIG. 8D shows a comparison of two-step RT-PCR systems with a random primer, in accordance with embodiments. FIG. 8E shows a comparison of two-step RT-PCR systems with a gene-specific primer, in accordance with embodiments. FIG. 8F shows an evaluation of an assay master mix, in accordance with embodiments. FIG. 8G shows an evaluation of an assay master mix, in accordance with embodiments.

FIG. 9 shows an evaluation of polymerase chain reaction conditions, in accordance with embodiments.

FIG. 10 shows an evaluation of polymerase chain reaction conditions, in accordance with embodiments.

FIG. 11A shows an evaluation of polymerase chain reaction parameters on genome length, in accordance with embodiments. FIG. 11B shows an evaluation of polymerase chain reaction parameters on average genome coverage, in accordance with embodiments. FIG. 11C shows an evaluation of polymerase chain reaction parameters on genome length, in accordance with embodiments.

FIG. 12A shows an evaluation of an equimolar primer mix, in accordance with embodiments. FIG. 12B shows an evaluation of an equimolar primer mix, in accordance with embodiments. FIG. 12C shows an evaluation of a remixed primer mix, in accordance with embodiments. FIG. 12D shows an evaluation of a remixed primer mix, in accordance with embodiments.

FIG. 13A shows a graph of read depth for nucleic acid analysis protocols that do not use 5′-biotinylated primers, in accordance with embodiments. FIG. 13B shows a graph of read depth for nucleic acid analysis protocols that use 5′-biotinylated primers, in accordance with embodiments.

FIG. 13C shows a comparison of the effects of 5′-biotinylation of primers on amplification representation, in accordance with embodiments.

FIG. 14 shows a graph of the impact of touchdown PCR on undetermined bases in nucleic acid analysis, in accordance with embodiments.

FIG. 15 shows a graph of genome coverage against read depth for amplicons of various length, in accordance with embodiments.

FIG. 16A shows effects of presence or absence of PEG in tegmented nucleic acid molecules, in accordance with embodiments. FIG. 16B shows a graph of reconstructed genomes with respect to the presence or absence of PEG in tegmented nucleic acid molecules, in accordance with embodiments. FIG. 16C shows a graph of uncalled bases for samples comprising nucleic acids tegmented with or without PEG, in accordance with embodiments.

FIG. 17A shows a graph of sequencing read depth for un-normalized sequencing libraries, in accordance with embodiments. FIG. 17B shows a graph of sequencing read depth for normalized sequencing libraries, in accordance with embodiments.

FIG. 18A shows a graph of genome reconstruction success with and without hybrid capture, in accordance with embodiments. FIG. 18B shows a graph of genome length with and without hybrid capture, in accordance with embodiments. FIG. 18C shows a graph of uncalled bases with and without hybrid capture, in accordance with embodiments.

FIG. 19 shows a graph of genome reconstruction success without a SpeedVac step (“beads”, left) or with a SpeedVac step (“SpeedVac”, right), in accordance with embodiments.

FIG. 20 shows a chart of genome reconstructions attained using systems and methods described herein, in accordance with embodiments.

FIG. 21A shows an image of a visualization of prevalence and spread within a geographical area comprising a population of subjects, in accordance with embodiments.

FIG. 21B shows a histogram of the prevalence of individual variants of concern in a population of subjects over time, in accordance with embodiments.

FIG. 22 shows a diagram of a computer system, in accordance with embodiments.

FIG. 23 shows a block diagram of a computer system, in accordance with embodiments.

FIG. 24 shows a diagram of network connections and configurations, in accordance with embodiments.

FIG. 25 shows a block diagram of a multiprocessor computer system using a shared virtual address memory space, in accordance with embodiments.

FIG. 26 shows method steps for performing nucleic acid analysis, in accordance with embodiments.

FIG. 27 shows method steps for performing nucleic acid analysis, in accordance with embodiments.

FIG. 28 shows method steps for performing nucleic acid analysis, in accordance with embodiments.

FIG. 29A shows a diagram illustrating an exemplary workflow of hitpicking. FIG. 29B shows a diagram illustrating reagent filling. FIG. 29C shows a diagram illustrating an exemplary workflow of sample processing. FIG. 29D shows a diagram illustrating an exemplary workflow of barcoding and sequencing.

### DETAILED DESCRIPTION

Provided herein are methods and systems useful for high-throughput molecular analysis. In particular, methods and systems disclosed herein can be useful for performing efficient and accurate molecular analysis of pathogens (e.g., contagious pathogens, such as severe acute respiratory syndrome coronavirus 2 (SARS-CoV-2, or “Covid-19”), which may give rise to a condition in a subject, such as an infectious disease) in a population. In many cases, methods and systems described herein can be useful for management, mitigation, and/or eradication of a pathogen from a population of subjects (e.g., patients). In many cases, successful management, mitigation, and/or eradication of a pathogen (e.g., a pathogen, such as SARS-CoV-2) from a population of subjects can depend on (1) prompt and reliable identification of individuals who have been exposed to the pathogen and/or who have contracted an infectious disease resulting from exposure to the pathogen and/or (2) prompt and reliable identification of new variants (e.g., mutants) of the pathogen within the population. This is especially true in urban settings, where the speed of transmission of pathogens among subjects in a population and/or creation of new pathogen variants within the population can be at the highest, for example, because of inherently close person-to-person proximity and increased reliance on shared resources, such as public transportation.

In some cases, heterogeneous implementation of prevention and response strategies and the speed and accuracy with which a given infected individual can be identified can be confounding factors for identifying or monitoring novel pathogen (e.g., new viral variants of concern, a novel cellular pathogen (such as a bacterium), or a pathogen derived from a cellular pathogen) in a population. In some cases, whole-genome sequencing against one or more genomes of an infectious agent (e.g. contagious pathogen, for instance, a virus such as Severe Acute Respiratory Syndrome Coronavirus 2 (SARS-CoV-2, or Covid-19) or a bacterium) can be included in strategies (e.g., comprising system, methods, devices, and/or kits described herein) for monitoring the emergence of pathogen variants (e.g., viral variants or bacterial strains) with increased virulence, transmissibility, and immune or vaccine evasion (or antibiotic resistance) and/or tracking their lineages, which can be important steps in blunting dangerous widespread public health events. There currently exists a need in the field for new and improved methods and systems to facilitate efficient and reliable monitoring of the progression and spread of a pathogen within a population, which can be heavily influenced by heterogeneity in the implementation of prevention and response strategies and/or the speed and accuracy at which a given infected individual is identified by a certain test. Speed, accuracy, reliability, and scalability can each be important for successfully identifying, monitoring, and/or combating the spread of an infectious agent (e.g., a virus or other contagious pathogen, such as a bacterium) within a given population as a whole. Accordingly, there exists an opportunity to increase the efficacy and agility of new and existing testing and treatment strategies, which can be predicated on the procurement of accurate data representative of entire populations of subjects, such as the population of a major metropolitan area.

In various aspects, methods and systems disclosed herein can provide highly reliable, highly efficient, and/or readily scalable means for identifying and tracking known and novel pathogen variants (e.g., variants of concern or “VOCs”) within a population ranging in size from a major metropolitan of millions of individuals to subpopulations (e.g., of hundreds or thousands of individuals) within a population (e.g., wherein the subpopulation may be located within a defined geographical area or wherein the subpopulation, which may or may not be geographically interspersed within a larger population, shares a demographic or genetic predisposition for vulnerability to the pathogen, for instance, such as elderly citizens living in retirement homes located throughout a metropolitan area). In some cases, utilization of methods and systems disclosed herein can increase the speed and/or accuracy of pathogen genome sequencing, which can, e.g., improve efficiency and/or accuracy of genomic surveillance of pathogens within a population. In some aspects, methods and systems disclosed herein can comprise one or more of: (1) an improved transportation and/or storage condition, e.g., for assaying a biological sample (or derivative thereof, such as a polymerase chain reaction (PCR) amplification product (e.g., amplicon)) at a first location (for instance, wherein the biological sample was collected at a second location), (2) an improved assay condition involved in determining a characteristic of a biological sample (e.g., determining the presence and/or sequence identity of all or a portion of a genome of a pathogen present in the biological sample), and/or (3) a method or system capable of visualizing and/or analyzing levels and/or transmission characteristics (e.g., speed of transmission and/or quantitative or qualitative assessment of demographic or genetic characteristics of affected subjects) of one or more variants of a pathogen within a population. In some cases, a method, composition, or system disclosed herein can comprise analysis of a biological sample or derivative thereof (e.g., a polymerase chain reaction (PCR) amplicon of a biological sample or portion thereof) by genome sequencing. In some cases, methods, systems, compositions, and/or devices described herein can provide means for synthesizing polynucleotides using an efficient RNA polymerase enzyme, microfluidics, enhanced kinetics, and reduced off-target effects.

Disclosed herein are process improvements, optimizations, and automation of a scalable, high-throughput, low-cost sequencing system and method, and its contribution to metropolitan-wide surveillance of the spread of VOCs. Some embodiments described herein demonstrate system configurations, system components, and/or method steps useful to maximize scalability, while preserving data quality, which can be critical for epidemiological study and resulting public health measures. Some example embodiments reflect the current and future utility of this system and method in the fight against the current COVID-19 (e.g., pandemic and the broad adoption of genomic biosurveillance of other pathogens with the potential to spawn new pandemics which may threaten public life and commerce. The COVID-19 crisis has highlighted the role of prevention as the most critical element of pandemic response. The tools utilized during this time will be essential to mitigate the likelihood and severity of future pandemics.

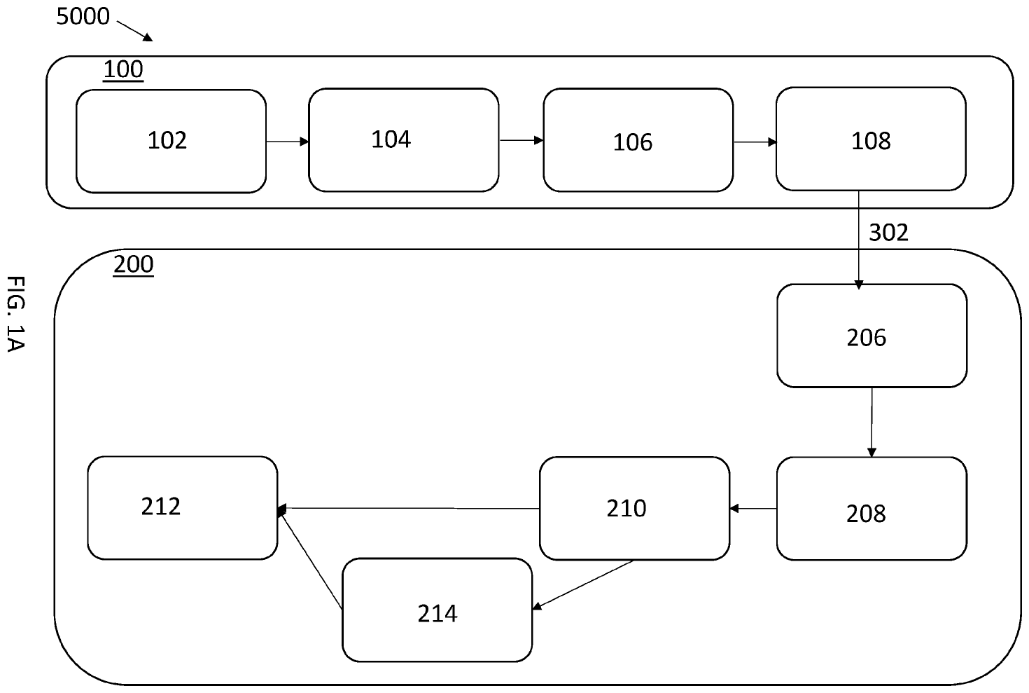

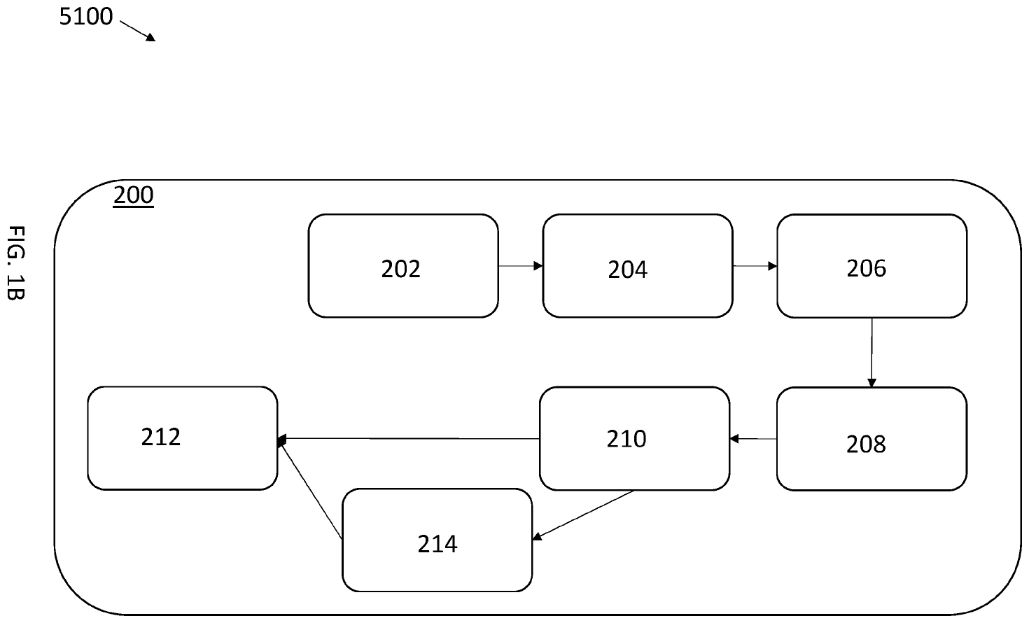

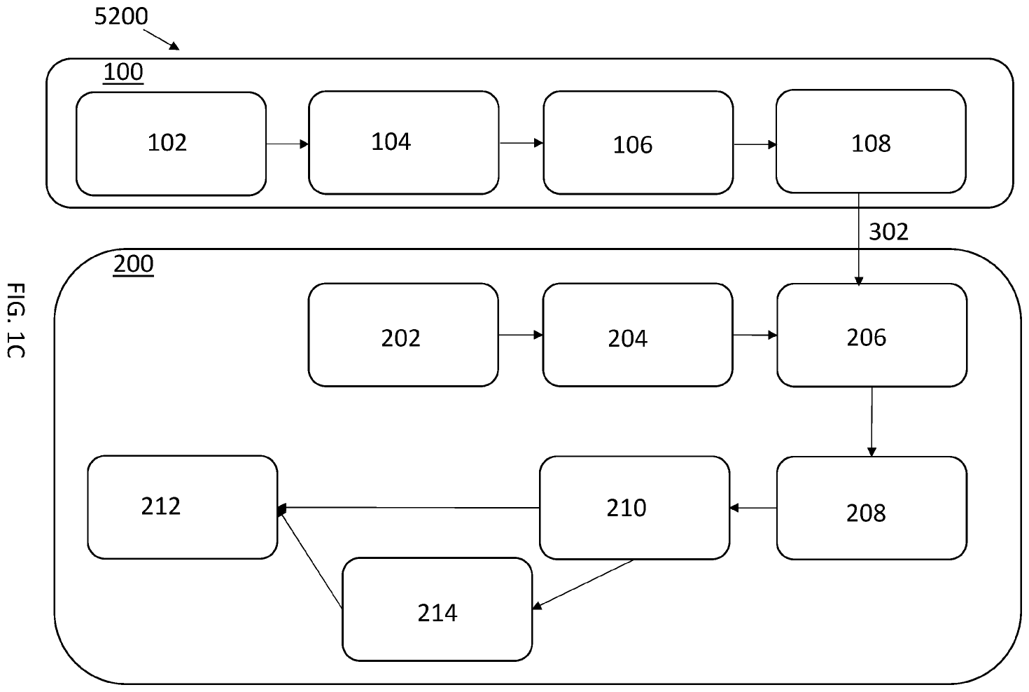

In some cases, systems, devices, and methods disclosed herein can render a response to the presence of an infectious agent in a population faster and more efficient, for example, because the end-to-end process of obtaining samples, detecting viral or bacterial nucleic acids, and sequencing genomes can be vertically integrated or because one or more individual steps of such processes or one or more components of a system useful in implementing such a process can be less time-intensive or more informative. In some cases, RNA samples found to be positive for an infectious agent (e.g., SARS-CoV-2) can be transported and sequenced from the second location (FIG. 1A, FIG. 1C) for sequencing in a separate facility (e.g., at the first location 100 or another location). In some cases, a fully integrated workflow can be completed in a timeframe as rapid as 6 hours, e.g., for processes or systems comprising qPCR-based analysis or diagnosis. In some cases, a method or system described herein can allow diagnosis of specimens and/or a subject from which an analyzed specimen is derived in under 24 hours after receipt of a sample, e.g., at a testing location, and/or can allow for determining a nucleotide sequence of a sample and/or a genomic reconstruction of a pathogen (e.g., via sequencing) within 28 hours or less from the time of receipt of a sample at a testing facility. In some cases, systems and methods described herein allow for analysis of 1 to 45,000 samples per day, 10,000 to 45,000 samples per day, 20,000 to 45,000 samples per day, 30,000 to 45,000 samples per day, 40,000 to 45,000 samples per day, or more than 45,000 samples per day (e.g., wherein end-to-end analysis of one or more (e.g., all of or a majority of) individual samples may be completed in less than 48 hours, less than 36 hours, less than 30 hours, or less than 24 hours). In some cases, infectious agent genomes (e.g., over 400,000 genomes per year) can be subjected to strict quality control criteria and can be provided to shared resource databases, such as the Global Initiative on Sharing Avian Influenza Data (GISAID). Systems and methods described herein can allow for the rapid identification of novel variants of concern at ports of entry (e.g. an airport) or via community spread.

### Systems

Systems disclosed herein can increase the efficiency and/or accuracy of nucleic acid analysis (e.g., comprising polymerase chain reaction amplification, nucleic acid sequencing, and/or genome biosurveillance of a population).

A system described herein can comprise one or more analysis modules (e.g., 106, 206, 212, as shown in FIG. 1A, FIG. 1B, and/or FIG. 1C). In some cases, an analysis module can be used to analyze one or more molecules (e.g., one or more nucleic acid molecules, such as one or more RNA molecules or populations thereof or one or more DNA molecules or populations thereof) of a sample derived from a subject. In some cases, analysis of a nucleic acid from a sample (e.g., using a system described herein) can be used to determine a sequence of the nucleic acid molecule. In some cases, a system described herein can be used to determine a prevalence or progression of a pathogen variant of interest having a nucleotide sequence of interest within a patient population or geographical area. In some cases, a nucleic acid molecule can be subjected to a polymerase chain reaction (PCR) assay (e.g., a qPCR assay or an RT-PCR assay) using one or more components of a system described herein (e.g., one or more analysis modules), as described herein. In some cases, a one or more parameters of a polymerase chain reaction assay protocol or reagent can be adjusted or modified (e.g., as described herein), for instance, to increase the speed and/or the accuracy of the nucleic acid analysis assay. For example, a primer used in a PCR assay can be modified by being biotinylated at a 5′ end of the primer.

### Polymerase Chain Reaction (PCR) System Configurations

In some cases, an analysis module can be useful in determining a nucleotide sequence of one or more nucleotide molecules, one or more populations of nucleotide molecules, and/or one or more subsets of a population of nucleotide molecules of a sample derived from a subject. In some cases, an analysis module can produce data useful in determining the presence or absence of a nucleotide sequence of interest (e.g., corresponding to a variant of concern (VOC), for instance, of a pathogen (e.g., an infectious agent) described herein) in a sample obtained from a subject.

For amplification of the SARS-CoV-2 genome, the Midnight primer set and protocol was chosen as a starting point to reduce the proportion of the genome to which primers anneal, mitigating the risk of fragment dropouts due to accumulation of mutations in those regions. This protocol calls for using a two-step RT-PCR protocol utilizing the LunaScript® RT SuperMix Kit for RT and Q5® Hot Start HF 2× Master Mix for PCR amplification of the cDNA.

In some cases, a PCR assay can comprise the use of a tiling primer. In some cases, a tiling primer can be from 200 base pairs (bp) in length to 1500 base pairs in length. In some cases, a tiling primer can be from 200 bp to 400 bp, from 400 bp to 500 bp, from 500 bp to 600 bp, from 600 bp to 700 bp, from 700 bp to 800 bp, from 700 bp to 800 bp, from 800 bp to 900 bp, from 900 bp to 1000 bp, from 1000 bp to 1100 bp, from 1100 bp to 1200 bp, from 1200 bp to 1300 bp, from 1300 bp to 1400 bp, from 1400 bp to 1500 bp, or greater than 1500 bp in length.

As described above, a one-step RT-PCR kit can offer some advantages over two-step kits and protocols, including reduction of labor, reduction of cost, reduction of pipeline runtime, and, potentially, an increase in performance.

While the Takara one-step kit is expected to have a slightly higher error-rate compared to the two-step protocol due to the lack of a super high fidelity proofreading DNA polymerase, since these mutations will occur randomly through the genome, and the same mutation is not likely to occur across the many consensus genomes generated, they do little to compromise the overall quality of the genomic data generated. Given the savings in cost, labor, and time, the greater number of genomes constructed, and the substantially higher average quality and length of the genomes produced with the Takara one-step kit, the sequencing pipeline was converted to utilize this kit moving forward.

### RT-PCR Cycling Parameters

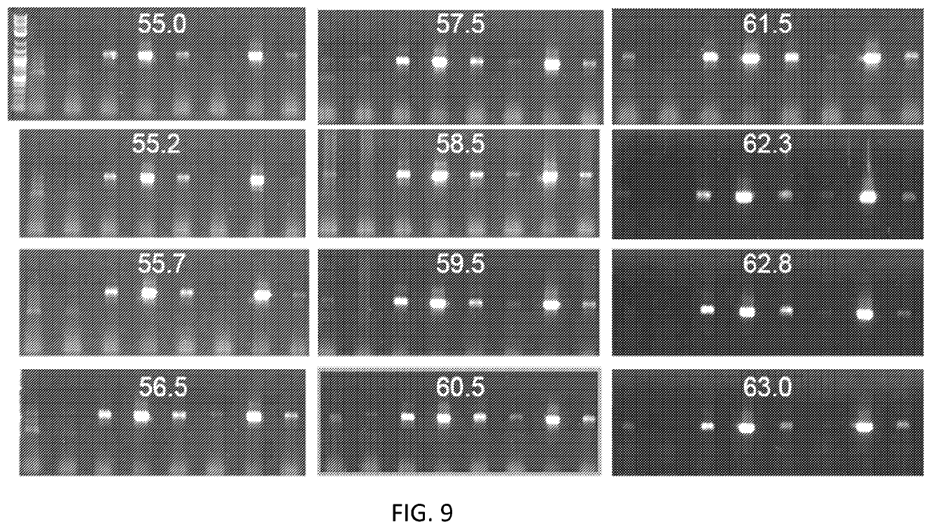

In some cases, the runtime of an amplification analysis protocol can be reduced while maintaining system performance, example embodiments optimize the PCR cycling parameters. In some cases, a temperature gradient can be performed during the elongation phase of the RT-PCR reaction to determine the optimal extension temperature. An extension temperature of from 60-65° C. can improve the quality and consistency of RT-PCR band production from analyzed nucleic acid populations while minimizing off-target product formation (FIG. 9). In some embodiments, an elongation temperature can be from about 60° C. to about 65° C. For example, an elongation temperature can be about 60° C., 60.5° C., 61° C., 61.5° C., 62° C., 62.5° C., 63° C., 63.5° C., 64° C., 64.5° C., or about 65° C. For example, an elongation temperature can be from about 60° C. to about 60.5° C., from about 60.5° C. to about 61° C., from about 61° C. to about 61.5° C., from about 61.5° C. to about 62° C., from about 62° C. to about 62.5° C., from about 62.5° C. to about 63° C., from about 63° C. to about 63.5° C., from about 63.5° C. to about 64° C., from about 64° C. to about 64.5° C., or from about 64.5° C. to about 65° C. Some example embodiments can comprise minimizing the time of the extension phase of the RT-PCR protocol, for instance, wherein protocol time is minimized without compromising the ability to produce products from even samples with a high Ct value (e.g., wherein an assayed nucleic acid population is present in the sample but in low concentration). In some cases, an extension time can be 3 minutes or greater (e.g., to avoid loss of multiplexed PCR product). In some cases, an extension time can be 3 minutes or less, for instance to avoid production of excessive amounts of large molecular weight off-target amplification products. In some embodiments, an extension time can be from about 0.5 minute to about 5 minutes. In some embodiments, an extension time can be about 0.5 minute, 1 minute, 1.5 minutes, 2 minutes, 2.5 minutes, 3 minutes, 3.5 minutes, 4 minutes, 4.5 minutes, or about 5 minutes. In some embodiments, an extension time can be from about 0.5 minute to about 1 minute, from about 1 minute to about 1.5 minutes, from about 1.5 minutes to about 2 minutes, from about 2 minutes to about 2.5 minutes, from about 2.5 minutes to about 3 minutes, from about 3 minutes to about 3.5 minutes, from about 3.5 minutes to about 4 minutes, from about 4 minutes to about 4.5 minutes, or from about 4.5 minutes to about 5 minutes. In some cases, an extension time can be 3 minutes. In some cases, a two-step cycling protocol for multiplexed PCR of the SARS-CoV-2 genome can be implemented to take advantage of these benefits. In some embodiments, a Ct value can be about 30, 31, 32, 33, 34, 35, 36, or about 37. In some embodiments, a Ct value can be below or no more than about 30, 31, 32, 33, 34, 35, 36, or about 37. In some embodiments, a Ct value can be above or higher than about 30, 31, 32, 33, 34, 35, 36, or about 37.

To further improve pipeline performance while reducing protocol runtime, we sought to optimize the PCR cycling parameters. First, a temperature gradient was performed during the elongation cycle of the RT-PCR reaction utilizing the Takara PrimeScript kit to determine the optimal extension temperature. The extension temperature of 61° C. was found to be optimal for producing an RT-PCR band from the most samples while minimizing off-target product formation. We next sought to minimize the extension time without compromising the ability to produce products from marginal, high Ct samples. We found that extension times less than 3 minutes resulted in loss of multiplexed PCR product from some samples, while extension times longer than 3 minutes resulted in the formation of large molecular weight off-products (data not shown). These findings were incorporated into the final two-step cycling protocol for RT-PCR of the SARS-CoV-2 genome.

Next, the volume of sample RNA added to the RT-PCR reaction was optimized. While increasing total RNA in the reaction should intuitively improve RT-PCR performance of low-concentration samples, residual contaminants from the RNA extraction procedure can also inhibit RT-PCR. Therefore, it was necessary to determine the RNA volume that would maximize the RNA input without compromising RT-PCR performance. When tested on STAT samples that were directly received and extracted at the LIC site using Kingfisher™ Flex (Thermo Scientific), there was a clear difference in the number of uncalled bases, average coverage, and genome length for different input volumes, and optimal results were observed with higher input volume of 5 μL. The differing RNA input results between the STAT and the NYC line samples can likely be attributed to some combination of extraction mechanisms, difference in elution volumes, and differences in sample handling and timing before RT-PCR. Consequently, this parameter should be optimized for any new genome sequencing operation which may use alternative extraction methods or instruments.

In some cases, a system or method described herein can include the use of a touchdown PCR protocol or a portion thereof.

### RNA Input Volume Optimization for RT-PCR

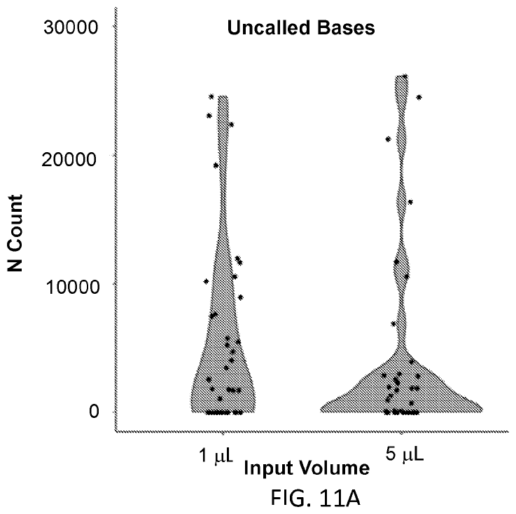

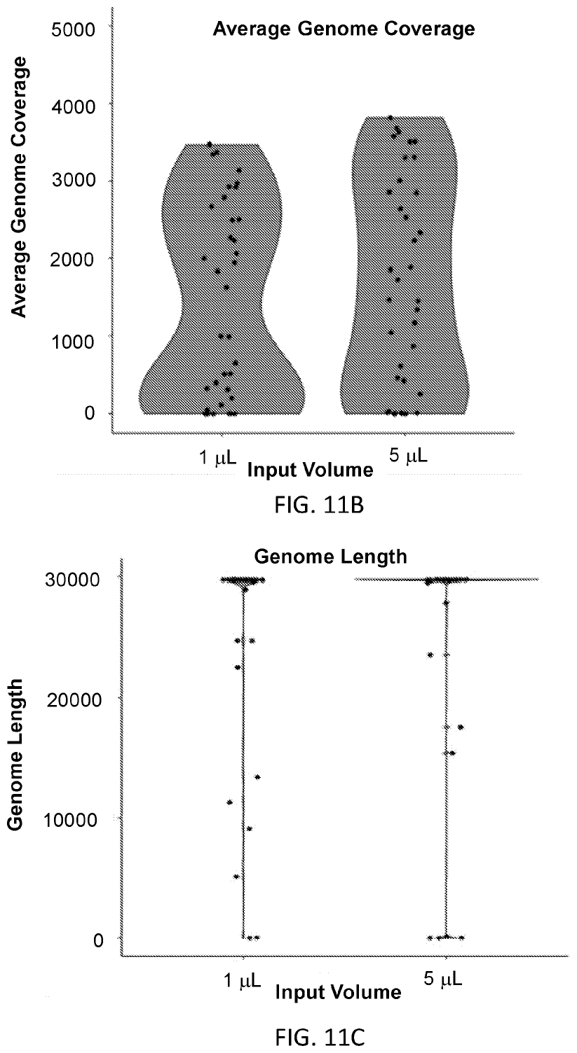

In some cases, it may be beneficial or necessary to determine the RNA volume that would maximize the RNA input without increasing residual contaminants to the point that can potentially inhibit the PCR reactions. In some cases, an input volume can be at least 0.5 μL. For example, an input volume can be about 0.5 μL, 1 μL, 1.5 μL, 2 μL, 2.5 μL, 3 μL, 3.5 μL, 4 μL, 4.5 μL, 5 μL, 5.5 L, 6 μL, 6.5 μL, 7 μL, 7.5 μL, 8 μL, 8.5 μL, 9 μL, 9.5 μL, or about 10 μL. In some cases (e.g., when using STAT samples that are directly received and extracted using a ThermoFisher® Kingfisher™ Flex system), improvements to the number of uncalled bases (FIG. 11A), average coverage (FIG. 11B), and genome length (FIG. 11C) for different input volumes may result from the use of a higher input volume (e.g., of 5 μL, for example, as compared to 1 μL). To assess the impact of input volume on reconstruction rate in our pipeline, 37 independent SARS-CoV-2 positive patient samples were sequenced each using 1 μL or 5 μL as template. Of these samples, 18 successfully reconstructed when using 1 μL and 25 reconstructed when using 5 μL. This difference of an additional 7 genomes is indicated in the “Saved” row and suggests an approximate improvement of ˜19%, though small sample size limits precise determination of this value. Table 1 shows experimental results comparing the effect of sample input volume on sequencing read percentages. The data shows that a larger volume (5 μL) of sample results in a greater amount of successful sequencing reads (e.g., as compared to 1 μL) (data indicates number of samples out of 37 samples tested for each condition; 7 samples (“saved”) were held out of analysis.

TABLE 1
The effect of sample input volume on sequencing read percentages

| Volume | 1 μL | 5 μL |
|---|---|---|
| TRUE | 18 | 25 |
| FALSE | 19 | 12 |

| | |
|---|---|
| Saved | 7 |

### Polymerase Chain Reaction (PCR) Reagents

In some cases, an analysis module can be useful in determining a nucleotide sequence of one or more nucleotide molecules, one or more populations of nucleotide molecules, and/or one or more subsets of a population of nucleotide molecules of a sample derived from a subject. In some cases, an analysis module can produce data useful in determining the presence or absence of a nucleotide sequence of interest (e.g., corresponding to a variant of concern (VOC), for instance, of a pathogen (e.g., an infectious agent) described herein) in a sample obtained from a subject.

### Primer Modification

Systems and methods described herein can comprise one or more primers. In some cases, a primer can comprise an oligonucleotide. In some cases, a primer can be modified. For example, modification of a primer can produced a modified primer. In some cases, a modification to a primer can comprise a modification to a 5′ end of the primer (e.g., a 5′ modification). In some cases, a 5′ end modification can comprise one or more molecules or a portion thereof (e.g., one or more of a polynucleotide or portion thereof, a carbohydrate or portion thereof, or an amino acid or portion thereof, or a small molecule). In some cases, a primer modification can comprise addition of one or more of a modified base, a linker, a non-fluorescent conjugate, a fluorescent dye, a 5′ terminal cap, a backbone, a dimer, a trimer, a quencher, a spacer, or a terminal phosphate.

In some cases, a primer modification can comprise a non-fluorescent conjugate. In some cases, a non-fluorescent conjugate can comprise biotinylation. In some cases, biotinylation of an oligonucleotide (e.g., a primer) can comprise addition of dT-biotin, 3′ biotin BB, biotin, biotin TEG, dual biotin, PC biotin, desthiobiotin TEG, or biotin BB to the oligonucleotide (e.g., to a 5′ end of a primer).

In some cases, a modified base can comprise a 2′-O-methyl RNA analog, a DNA purine (e.g., adenine or guanine) or analog thereof, an inverted base, an RNA analog, a sugar analog, a DNA pyrimidine (e.g., cytosine, methylcytosine, hydroxymethylcytosine, or uracil) or an analog thereof, a universal base, or a wobble base.

In some cases, a primer modification can comprise a 2′-O-methyl RNA analog. In some cases, a 2′-O-methyl RNA analog can comprise a 2,6-Diaminopurine-2′-O-methylriboside, 2-Aminopurine-2′-O-methylriboside, 3-Deaza-5-Aza-2′-O-methylcytidine, 5-Methyl-2′-O-Methylcytidine, 5-Bromo-2′-O-methyluridine, 5-Fluoro-2′-O-Methyluridine, 5-Fluoro-4-O-TMP-2′-O-Methyluridine, or 5-Methyl-2′-O-Methylthymidine.

In some cases, a primer modification can comprise a DNA purine. In some cases, a DNA purine can comprise 8-Bromo-2′-deoxyadenosine, 7-Deaza-2′-deoxyadenosine, 2,6-Diaminopurine-2′-deoxyriboside, Etheno-2′-deoxyadenosine, N6-Methyl-2′-deoxyadenosine, 8-Oxo-2′-deoxyadenosine, 8-Amino-2′-deoxyadenosine, 7-Deaza-8-aza-2′-deoxyadenosine, 8-5′(5'S)-Cyclo-2′-deoxyadenosine, N1-Methyl-2′-dA, 2-Aminopurine-2′-deoxyriboside, 5-Formylindole, 8-Bromo-2′-deoxyguanosine, 7-Deaza-2′-deoxyguanosine, 7-Deaza-2′-deoxyxanthosine, O6-Methyl-2′-deoxyguanosine, 8-Oxo-2′-deoxyguanosine, 6-Thio-2′-deoxyguanosine, 8-Amino-2′-deoxyguanosine, 8-Deuterated-2′-deoxyguanosine, 2′-Deoxythienoguanosine, O6-Phenyl-2′deoxyinosine, 2′ Deoxynebularine, K-2′-deoxyribose, or 5-Nitroindole-2′-deoxyriboside.

In some cases, a primer modification can comprise an inverted base. In some cases, an inverted base can comprise 3′-3′ Inverted Adenosine, 5′-5′ Inverted Adenosine, 3′-3′ Inverted Adenosine, 5′-5′ Inverted Adenosine, 5′-5′ Inverted Cytosine, 3′-3′ Inverted Cytidine, 3′-3′ Inverted Cytidine, 5′-5′ Inverted Cytosine, 5′-5′ Inverted Guanosine, 3′-3′ Inverted Guanosine, 3′-3′ Inverted Guanosine, 5′-5′ Inverted Guanine, 5′-5′ Inverted Thymidine, 3′-3′ Inverted Thymidine, 3′-3′ Inverted Thymidine, or 5′-5′ Inverted Thymidine.

In some cases, a primer modification can comprise an RNA analog. In some cases, an RNA analog can comprise 2,6-Diaminopurine-riboside, puromycin, 1-Methyladenosine, N6-Methyl-A, 2-Aminopurine-riboside, 5-Methylcytidine, Pyrrolocytidine, Thienocytidine, Thienoguanosine, 5-Bromouridine, 5-Iodouridine, 5-Methyluridine, 4-Thiouridine, Pseudouridine, Thienouridine, 1-Methylpseudouridine, or 5,6-Dihydrouridine.

In some cases, a primer modification can comprise a sugar analog. In some cases, a sugar analog can comprise 3′-Deoxyadenosine, 2′,3′-Dideoxyadenosine, OXP-protected 2′ dA, Aracytidine, 3′-Deoxycytidine, 2′,3′-Dideoxycytidine, 2′,3′-Dideoxycytidine (3′), OXP-protected 2′ dC, 3′-Deoxyguanosine, 2′,3′-Dideoxyguanosine, OXP-protected 2′ dG, 5′-Iodothymidine, 5′-O-Methylthymidine, 3′-Deoxythymidine, 2′,3′-Dideoxythymidine, or OXP-protected 2′ dT.

In some cases, a primer modification can comprise a DNA pyrimidine. In some cases, a DNA pyrimidine can comprise 5-Bromo-2′-deoxycytidine, N4-Ethyl-2′-deoxycytidine, 5-Iodo-2′-deoxycytidine, 5-Methyl-2′-deoxycytidine, 5-Propynyl-2′-deoxycytidine, 5-Hydroxy-2′-deoxycytidine, Pyrrolo-2′-deoxycytidine, 5-Hydroxymethyl-2′-deoxycytidine, 5-Carboxy-2′-deoxycytidine, 5-Formyl-2′-deoxycytidine, N4-Methyl-2′-dC, 5′ Aminothymidine, 5-Bromo-2′-deoxyuridine, 5-(Carboxy)vinyl-2′-deoxyuridine, 2′-Deoxypseudouridine, 2′-Deoxyuridine, 5,6-Dihydrothymidine, 5,6-Dihydro-2′-deoxyuridine, 5-Fluoro-2′-deoxyuridine, 5-Hydroxymethyl-2′-deoxyuridine, 5-Hydroxy-2′-deoxyuridine, 5-Iodo-2′-deoxyuridine, 04-Methylthymidine, 5-Propynyl-2′-deoxyuridine, 4-Thio-2′-deoxyuridine, 2-Thiothymidine, 4-Thiothymidine, 6-O-(TMP)-5-F-2′-deoxyuridine, C4-(1,2,4-Triazol-1-yl)-2′-deoxyuridine, 5-(C2-EDTA)-2′-deoxyuridine, Thymidine Glycol, dT-Ferrocene, or 5-(1-Pyrenylethynyl)-2′-deoxyuridine.

In some cases, a primer modification can comprise a Universal base or wobble site. In some cases, a universal base or wobble site can comprise 5-Methyl-2′-deoxyisocytidine, 2′-Deoxyisoguanosine, Inosine, 2′-Deoxyinosine, 2′-O-Methylinosine, 3-Nitropyrrole-2′-deoxyribose, P-2′-deoxyribose, Pyrrolidine, K-2′-deoxyribose, P-2′-deoxyribose, or a wobble site.

In some cases, a primer modification can comprise a linker. In some cases, a linker can comprise an amino linker, a carboxyl linker, or a thiol linker.

In some cases, a primer modification can comprise an amino linker. In some cases, an amino linker can comprise a 2′-Deoxyadenosine-8-C6 Amino Linker, a 2′-Deoxycytidine-5-C6 Amino Linker, a 2′-Deoxyguanosine-N2-C6 Amino Linker, a Thymidine-5-C2 Amino Linker, a Uridine-C6-Amino Linker, a Thymidine-5-C6 Amino Linker, a 3′ C3 Amino Linker, a 5′ C6 Amino Linker, a 5′ C12 Amino Linker, a 3′ C6 Amino Linker, a 3′ C7 Amino Linker, a 5′ PC Amino Linker, a 2′-Deoxycytidine-5-C6 Amino Linker, an Amino-Modifier Serinol, or a Thymidine-5-C6 Amino Linker.

In some cases, a primer modification can comprise a carboxyl linker. In some cases, a carboxyl linker can comprise a dT-carboxy linker or a DADE linker.

In some cases, a primer modification can comprise a thiol linker. In some cases, a thiol linker can comprise a 5′ C6 Disulfide Linker, a 3′ C3 Disulfide Linker, a 3′ C6 Disulfide Linker, a Dithiol Linker, a Dithiol Linker, a C3 Thiol Linker, a C6 Thiol Linker, or a C6 Thiol Linker.

In some cases, a primer modification can comprise a PC linker, 3′ Glyceryl, 5′ 4-formylbenzamide, 5′ maleimide, 5′ hexnyl, C2 aldehyde, dithiol serinol, doubler, or trebler.

In some cases, a primer modification can comprise Cholesteryl TEG, DNP-X, Psoralen C2, DNP TEG, Psoralen C6, Acridine, Abasic, Azobenzene, DOTA, TINA, Zip Nucleic Acid, Cholesteryl TEG, Acridine, Digoxigenin.

In some cases, a primer modification can comprise a fluorescent dye. In some cases, a fluorescent dye can comprise a green or yellow fluorescent dye (e.g., Emₘₐₓ 492-585 nanometers), an orange fluorescent dye (e.g., Emₘₐₓ 586-647 nanometers), a red fluorescent dye (e.g., Emₘₐₓ 647-700 nanometers), or a violet or blue fluorescent dye (e.g., Emₘₐₓ 375-491 nanometers).

In some cases, a primer modification can comprise a green or yellow fluorescent dye. In some cases, a green or yellow fluorescent dye can comprise dT-FAM, dT-TAMRA, dT-FAM, BODIPY® 493/503, DTAF, 6-FAM SE, Dansyl-X, Oregon Green® 488, Oregon Green® 514, Rhodamine Green-X™, NBD-X, TET, 2′,4′,5′,7′-Tetrabromosufonefluorescein, 6-JOE, BODIPY® 530/550, HEX, Carboxyrhodamine 6G™, BODIPY® 558/568, PyMPO, Cyanine 3 CE, TAMRA-X, Rhodamine Red-X™, Alexa Fluor® 430, Alexa Fluor® 488, Alexa Fluor® 532, Alexa Fluor® 546, Alexa Fluor® 555, Cal Fluor® Gold 540, Alexa Fluor® 514, CAL Fluor® Orange 560, Quasar® 570, Biodesyl, 6-Carboxy-X-Rhodamine, 6-FAM Amidite, 6-TAMRA-X, Alexa Fluor® 488 Maleimide, Alexa Fluor® 532 Maleimide, Alexa Fluor® 546 Maleimide, Alexa Fluor® 555 Maleimide, Cyanine 3 SE, FAM, or TET (Tetrachlorofluorescein) SE.

In some cases, a primer modification can comprise an orange fluorescent dye. In some cases, an orange fluorescent dye can comprise BODIPY® 576/589, BODIPY® 581/591, Texas-Red®-X, BODIPY®-TR-X, Alexa Fluor® 568, Alexa Fluor® 594, CAL Fluor® Red 590, CAL Fluor® Red 610, Alexa Fluor® 610, CAL Fluor® Red 635, Alexa Fluor® 568 Maleimide, or Texas-Red-X Maleimide.

In some cases, a primer modification can comprise a red dye. In some cases, a red dye can comprise Cyanine 5 CE, Carboxynaphthofluorescein, Alexa Fluor® 633, Alexa Fluor® 647, Alexa Fluor® 660, Alexa Fluor® 680, Alexa Fluor® 700, Alexa Fluor® 750, Quasar® 670, IRDye® 650, IRDye® 680RD, IRDye® 750, IRDye® 800CW, IRDye® 700, IRDye® 800, Alexa Fluor® 647 Maleimide, Alexa Fluor® 750 Maleimide, Cyanine 5 SE, Hexachlorofluorescein (e.g., HEX)) SE, IRDye® 650 Maleimide, IRDye® 680RD Maleimide, IRDye® 750 Maleimide, or IRDye® 800CW Maleimide.

In some cases, a primer modification can comprise a violet or blue dye. In some cases, a violet or blue dye can comprise Pyrene, 7-Methoxycoumarin, Cascade Blue®, 7-Aminocoumarin-X, Pacific Blue®, Marina Blue®, Dimethylaminocoumarin, Alexa Fluor® 350, Alexa Fluor® 405, Alexa Fluor® 350 Maleimide.

In some cases, a primer modification can comprise a 5′ terminal cap. In some cases, a 5′ terminal cap can comprise 5′ adenylate, 5′ guanosine-trisphosphate cap, 5′ N⁷-methylguanosine triphosphate cap, or 5′ triphosphate cap.

In some cases, a primer modification can comprise a backbone molecule. In some cases, a backbone molecule can comprise DNA, RNA, a phosphorothioate, 2′O-Methyl RNA, methylphosphonate, 2′ Fluoro RNA, or a phosphodiester backbone.

In some cases, a primer modification can comprise a dimer or trimer. In some cases, a dimer or trimer can comprise a cis-syn thymidine dimer or codon trimer mix.

In some cases, a primer modification can comprise a quencher. In some cases, a quencher can comprise DABCYL SE, 3′-DABCYL, dT-DABCYL, QSY-7®, QSY-9®, QSY-21®, QSY-35®, Black Hole Quencher 1® Amidite, Black Hole Quencher 2® Amidite, IRDye® QC-1, Black Hole Quencher 1 ® SE, Black Hole Quencher 1®, Black Hole Quencher 2 ® SE, Black Hole Quencher 2®, QSY-7 ® Maleimide, Black Hole Quencher 3 ®, Black Hole Quencher 3 ® SE.

In some cases, a primer modification can comprise a spacer. In some cases, a spacer can comprise C3 Spacer, Spacer 9, C12 Spacer Spacer 18, dSpacer, C6 Spacer, rSpacer, PC Spacer, or C3 (Propyl) Spacer.

In some cases, a primer modification can comprise a terminal phosphate. In some cases, a terminal phosphate can comprise 5′ phosphate, 3′ phosphate, 3′ thiophosphate, or 5′ thiophosphate.

In some cases, a 5′ modification of a primer (e.g., 5′ biotinylation of the primer) can inhibit or (partially or completely) block 5′ to 3′ exonuclease activity. In some cases, 5′ modification of a primer (e.g., by addition of a molecule to the 5′ end of the primer, for instance, via biotinylation of the 5′ end of the primer) can improve the evenness of results of a nucleic acid analysis assay, as described herein.

### Analysis Modules

In some cases, an analysis module can be useful in determining a nucleotide sequence of one or more nucleotide molecules, one or more populations of nucleotide molecules, and/or one or more subsets of a population of nucleotide molecules of a sample derived from a subject. In some cases, an analysis module can produce data useful in determining the presence or absence of a nucleotide sequence of interest (e.g., corresponding to a variant of concern (VOC), for instance, of a pathogen (e.g., an infectious agent) described herein) in a sample obtained from a subject. In some cases, determining the presence or absence of a nucleotide sequence of interest can be useful in determining whether the subject has a pathogen (e.g., an infectious agent, such as a virus or other contagious pathogen, such as a bacterium). In some cases, determining the presence or absence of a nucleotide sequence of interest can be useful in identifying a sequence of interest, such as a novel sequence of interest or a known sequence of interest, (e.g., using a system or method described herein), which may, for example, comprise a novel variant of concern (VOC). In some cases, determination of the presence of a nucleotide sequence of interest in a sample derived from a subject can indicate that the subject has a pathogen comprising the nucleotide sequence of interest. In some cases, determination of the absence of a nucleotide sequence of interest in a sample derived from a subject can indicate that the subject does not have a pathogen comprising the nucleotide sequence of interest. In some cases, determination of the presence of a nucleotide sequence of interest in a sample derived from a subject can be useful in reconstructing a genome of a pathogen comprising the nucleotide sequence of interest. In some cases, a determination of the presence (or absence) of a nucleotide sequence of interest in a sample can be useful in generating an analysis (e.g., comprising a geographical map) of the presence, prevalence, and/or spread of a pathogen comprising the nucleotide sequence of interest in a community of individuals (e.g., comprising the subject from whom the sample was derived). In some cases, determining the presence (or absence) of a nucleotide sequence of interest in a sample can be useful in creating or modifying a method, strategy, or policy regarding mitigation or elimination of the pathogen (e.g., and/or its spread) within a community.

In some cases, an analysis module can be used to analyze a plurality of nucleic acid molecules (e.g., via molecular sequencing or molecular amplification analysis, for instance using an analysis module comprising a sequencing device 212). For example, (e.g., to determine a presence or absence of a nucleic acid sequence of interest or sequence identity of all or a portion of a nucleic acid sequence of interest within the plurality of nucleic acid molecules). In some cases, an analysis module 106, 206, 212 can be used to analyze a plurality of nucleic acids present in a biological sample obtained from a subject (e.g., a saliva sample, a blood sample, a urine sample, a cell lysate, and/or a tissue biopsy). In some cases, an analysis module 106, 206, 212 can be used to analyze a plurality of nucleic acids derived from one or more molecules (e.g., one or more nucleic acid molecules) present in a biological sample obtained from a subject. For example, an analysis module 106, 206 (e.g., comprising an amplification device, such as an amplification device configured to execute a one-step amplification protocol) can be used to analyze a polymerase chain reaction (PCR) amplification product (e.g., an amplicon) of all or a portion of one or more molecules (e.g., one or more nucleic acid molecules, e.g., RNA molecule(s) or DNA molecule(s) such as genomic DNA or cell-free (cfDNA) molecules) present in a biological sample obtained from a subject, for instance using nucleic acid amplification analysis, which may comprise determining whether an amplification cycle threshold (Ct) value for a nucleotide sequence of interest meets or exceeds a criterion (e.g., a hitpicking threshold value).

In some cases, incorporation of a one-step PCR amplification protocol into a method or system described herein can offer certain advantages over a method or system utilizing a two-step PCR amplification, e.g., as described herein. For example, use of a one-step PCR amplification protocol can increase speed of amplification steps and/or protocols and or the speed of a multiple-step method described herein (e.g., which may be implemented using a system described herein, in some cases). In some cases, use of a one-step PCR amplification protocol can reduce the contamination of a sample during the practice of a method or system described herein (e.g., compared to a method or system comprising a two-step PCR amplification protocol), for example, because a one-step PCR amplification protocol can reduce the number of steps or repetition of steps (e.g., which may comprise liquid and/or sample handling) involved in the practice of the method or system. In some cases, increased sequencing depth and/or coverage can be obtained using a method or system described herein comprising a one-step PCR amplification protocol (e.g., as compared to a method or system comprising a two-step PCR amplification protocol), for instance, because the increased speed of the sample preparation, sample processing, and or the PCR amplification assay(s) can allow for more time for sequencing assays, which can allow for sequencing assays to be run at a greater depth. In some cases, benefits of the use of a one-step PCR amplification protocol (e.g., as compared to a two-step PCR amplification protocol can be magnified by the scale at which the method or system is implemented. For instance, one or more benefits of a method or system described herein that utilizes a one-step PCR amplification protocol can be magnified or multiplied when the method or system is used to assay (e.g., to surveille or monitor) a larger patient population (e.g., a large municipality versus a specific portion of a small community). In some cases, the magnification of the benefit(s) of methods or systems utilizing one-step PCR amplification protocols (e.g., as described herein) can reduce the overall cost or cost per unit time of assaying a given patient population.

In some cases, a PCR amplification product derived from one or more nucleic acid molecules present in a biological sample can yield more accurate and/or more reproducible results when analyzed (e.g., via nucleic acid amplification analysis) with an analysis module than using the one or more nucleic acid molecules present in the biological sample, for example, because the PCR amplification product can be more stable in storage and/or transport, more plentiful and/or easier to prepare for analysis (e.g., during barcoding and adapter ligation) than nucleic acid molecules present in the biological sample. In some cases, data regarding the presence or absence of a sequence of interest in one or more nucleic acid molecules present in a biological sample obtained from a subject can be useful in determining whether the subject has been exposed to a pathogen of interest (e.g., a variant of concern (VOC) of a pathogen of interest), whether the subject has contracted a disease (e.g., an infectious disease resulting from exposure to the pathogen of interest, such as SARS-CoV-2), and/or the state of progression of a disease in the subject (e.g., an infectious disease resulting from exposure to the pathogen of interest). In some cases, accurate and efficient determination of the number of subjects in a population that have been exposed to a pathogen, that have contracted a disease (e.g., an infectious disease resulting from exposure to the pathogen of interest), and/or the state of progression of a disease (e.g., infectious disease) in one or more subjects of the population (e.g., using methods and systems disclosed herein) can allow for efficient and accurate surveillance of known and novel pathogens in the population.

In some cases, all or a portion of data produced by an analysis module (e.g., comprising one or more of an amplification cycle threshold (Ct) values and/or an alignment of a test nucleotide sequence with a reference genome or portion thereof) can be used as a basis for development or modification of a strategy for mitigating or preventing spread of an infectious agent.

An analysis module 106, 206, 212 can comprise a thermocycler, a luminescence detector, and/or a fluorescence detector (e.g., wherein the detector is coupled to a thermocycler, for example, to perform a polymerase chain reaction assay (e.g., comprising PCR analysis, such as, reverse transcriptase-PCR (RT-PCR) analysis), which may comprise a one-step PCR protocol, on a sample or derivative thereof), and/or a sequencer (e.g., a sequencer capable of performing first generation (e.g., Sanger sequencing), second generation (e.g., next generation sequencing (NGS)), or third generation (long-read sequencing) nucleic acid sequencing). In some cases, an analysis module 106, 206, 212 can be used to analyze a plurality of ribonucleic acid molecules (e.g., messenger RNA (mRNA)). In some cases, an analysis module 106, 206, 212 can be used to analyze a plurality of deoxyribonucleic acid molecules (e.g., fragmented or unfragmented genomic DNA or cell-free DNA (cfDNA)). In some cases, an analysis module (e.g., a sequencer 114) can be used to analyze (e.g., sequence) a first population of nucleic acid molecules (e.g., polynucleotides, which may comprise RNA or DNA, such as cDNA molecules derived from one or more RNA or DNA molecules) present in a biological sample obtained from a subject (e.g., a saliva sample, a blood sample, a urine sample, a cell lysate, and/or a tissue biopsy). In some cases, an analysis module (e.g., a sequencer 114) can be used to analyze (e.g., sequence) a first population of nucleic acid molecules (e.g., polynucleotides, which may comprise RNA or DNA) present in a biological sample obtained from a subject (e.g., a saliva sample, a blood sample, a urine sample, a cell lysate, and/or a biopsy). In some cases, an analysis module can be used analyze a second population of nucleic acid molecules, which can comprise a plurality of nucleic acid molecules (e.g., PCR amplification products, “amplicons”) produced from a PCR amplification reaction.

In some cases, a PCR amplification protocol can comprise the use of a primer pool, such as an A400 primer pool or an A1200 primer pool (e.g., as described herein). In some cases, a PCR amplification protocol can comprise the use of a tiling primer that is longer than an A400 primer. In some cases, the use of a longer tiling PCR amplification protocols (e.g., comprising A1200 primers, for example, rather than A400 primers) can be advantageous for the construction of a genome of interest (e.g., a genome of a novel pathogen variant of concern). In some cases, increased tiling length can decrease the number of steps required to prepare and/or analyze nucleic acid molecules (or fragments or amplicons thereof). For example, increased tiling length can decrease the number of fragments produced during nucleic acid preparation, which can reduce the number of fragments that must be ligated to sequencing adapters (and, in some cases, the number of barcodes that must be tracked and deciphered). In some cases, such advantages can reduce the time required to analyze a plurality of nucleic acid molecules of a sample and/or the cost associated with analysis of a sample and/or associated with reconstruction of a novel genome of interest. In some cases, one or more of these advantages can be magnified or multiplied as a method or system is scaled up to assay (e.g., interrogate, surveille or monitor) a larger population.

In some cases, an analysis module (e.g., 106 and 206 in FIG. 1A, FIG. 1B, or FIG. 1C) can be used to perform polymerase chain reaction (PCR) amplification (e.g., reverse transcriptase polymerase chain reaction (RT-PCR)). In some cases, an analysis module can be used to perform a quantitative PCR assay (e.g., a qPCR assay, such as RT-qPCR). In some cases, an analysis module can comprise a thermocycler and a computer (e.g., a programmable computer comprising a memory and a processor capable of operating the thermocycler to execute a PCR, RT-PCR, or RT-qPCR program, e.g., as described herein). In some cases, PCR reagents and conditions 206 described herein (e.g., conditions used for PCR, RT-PCR, or RT-qPCR assays, which may help to improve overall efficiency and/or accuracy of genome and/or population biosurveillance, as described herein) can be used with one or more analysis modules described herein. In some cases, an analysis module can be used to create PCR amplification products (e.g., amplicons) from nucleic acid molecules (e.g., RNA molecules), for instance, after the nucleic acid molecules are isolated from a sample obtained from a patient. PCR amplification products generated by an analysis module (e.g., during a PCR, RT-PCR, or RT-qPCR assay, which may comprise the use of primer sets disclosed herein and/or thermocycle program steps disclosed herein) can be used in molecular sequencing (e.g., as described herein), for instance, after tagmentation, molecular barcoding, and/or library pooling (e.g., as described herein). In some cases, an analysis module comprising a thermocycler and a computer programmed to execute an RT-qPCR assay (e.g., as described herein) can be used to analyze the concentration and/or quality of nucleic acid molecules isolated from a sample (e.g., using PCR cycle threshold values (Ct), as described herein). In some cases, Ct values determined during RT-qPCR analysis of nucleic acid molecules isolated from a sample can be used to determine whether or not a nucleic acid molecule sequence of interest (e.g., a sequence present in a pathogen variant of concern (VOC)) was present in the sample (e.g., whether the sample was PCR-positive or PCR-negative).

In some cases, an analysis module (e.g., 206 in FIG. 1A) can be utilized at a second location 200, wherein the second location 200 is local to a sample collection site (e.g., using sample collection device 202) and remote from a first location 100 at which an analysis module for sequencing (e.g., a molecular sequencer) is located. The ability to perform RT-qPCR analysis at the second location 200 local to the collection site can be advantageous in determining whether a sample is PCR-positive or PCR-negative quickly. In some cases, determining whether a sample is PCR-positive or PCR-negative quickly (e.g., by avoiding the need for storing and/or transporting the sample or nucleic acids from the sample prior to an RT-qPCR assay) can help to avoid a decrease in nucleic acid integrity and/or confidence in determinations of whether a sample is PCR-positive or PCR-negative. As described herein, determinations of PCR-positivity or PCR-negativity can be used to inform hitpicking and processing of isolated nucleic acids, in some embodiments.

In some cases, a system, device, or method described herein can comprise sequencing one or more nucleic acid populations (e.g., one or more cDNA populations of a subset of a plurality of nucleic acid populations identified as hit(s) by amplification analysis), for instance to determine a nucleotide sequence of the one or more nucleic acid populations. In some cases, determining a nucleotide sequence of the one or more nucleic acid populations can comprise determining that a nucleotide sequence corresponding to a variant of concern (VOC) of an infectious agent (e.g., a virus or other contagious pathogen, such as a bacterium) is present in the nucleic acid population. In some cases, determining a nucleotide sequence of the one or more nucleic acid populations can comprise determining that a nucleotide sequence corresponding to a novel variant of concern (VOC) of an infectious agent (e.g., a virus or other contagious pathogen, such as a bacterium) is present in the nucleic acid population. In some cases, determining a nucleotide sequence of the one or more nucleic acid populations can comprise determining that a subject has an infectious agent (e.g., a virus or other contagious pathogen, such as a bacterium). In some cases, determining a nucleotide sequence of one or more nucleic acid populations corresponding to sample(s) obtained from one or more subjects can be used to determine an extent to which an infectious agent has spread within the population. In some cases, determining a nucleotide sequence of one or more nucleic acid populations corresponding to sample(s) obtained from one or more subjects can be used to determine a rate of mutation of an infectious agent (e.g., a virus or other contagious pathogen, such as a bacterium), for instance, by determining a rate at which novel variants of concern are identified within a population of subjects. In some cases, determining a nucleotide sequence of one or more nucleic acid populations corresponding to sample(s) obtained from one or more subjects can be used to determine a rate of transmission of an infectious agent (e.g., a virus or contagious pathogen, such as a bacterium) within a population of subjects, for instance, by determining a rate at which subjects within the population are identified as having the infectious agent.

### Processing Devices

In some cases, a system, device, or method described herein can comprise a processing device 108, 208, 210, 214. In some aspects, a processing device 108, 208, 210, 214 can improve the speed and/or accuracy of nucleic acid analysis described herein. For instance, a processing device 108, 208, 210, 214, which can comprise a fluid handling apparatus, a computer system (e.g., configured to execute a protocol for hitpicking and/or processing of one or more nucleic acid populations, and/or one or more reagents) can be configured to automatically perform hitpicking on a plurality of nucleic acid populations (e.g., based on plurality of Ct values corresponding to the respective populations of the plurality of nucleic acid populations). In some cases, a processing device 108, 208, 210, 214 can automatically transfer and/or spatially reformat or rearrange a plurality of nucleic acid populations into a container, e.g., based on a plurality of Ct values determined by analyzing the plurality of nucleic acid populations with an analysis module comprising an amplification device, which can be used to identify a subset of the plurality of nucleic acid populations that are positive for a sequence of interest (e.g., a subset of hits). In some cases, a processing device 108, 208, 210, 214 can also be used to process the selected subset of nucleic acid populations, for instance, in preparation for sequencing analysis using an analysis module comprising a sequencing device. In some cases, processing a nucleic acid population in preparation for sequencing can comprise addition (e.g., ligation) of a sequencing adapter and/or barcoding the nucleic acid population (e.g., with a unique molecular identifier).

In some cases, a liquid handling device can be or can comprise one or more processing devices 108, 208, 210, 214.

### Liquid Handling Systems

In some cases, a system disclosed herein can comprise a liquid handling system (e.g., an automated liquid handling system, such as an Opentrons OT-2 Liquid Handling Robot or Tecan). Automated liquid handling systems can be useful for improving the speed and accuracy with which samples or nucleic acids extracted from biological samples are processed. In particular, automated liquid handling systems can decrease the time required to complete various processes of methods and systems described herein, such as transferring samples from one container (e.g., a vial, a tube, or a well of a first well plate) to a second container (e.g., a well of a second well plate) and/or addition of assay reagents, and can reduce the likelihood of error (e.g., as compared to manual pipetting by a technician). In some cases, an automated liquid handling system can be used for steps involved in tagmentation and barcoding. For example, automated liquid handling systems can be used to efficiently transfer only nucleic acids extracted from PCR-positive samples to new multiwell plates from multiwell plates comprising both wells containing nucleic acids extracted from PCR-positive samples and wells containing nucleic acids extracted from PCR-negative samples that may have been transported from a location at which the nucleic acids were isolated (e.g., in order to consolidate the PCR-positive samples for more efficient processing). In some cases, in which it is necessary to transfer nucleic acids from a first multiwell plate having a first two-dimensional well format (e.g., a 96-well plate) to a multiwell plate having a second two-dimensional well format (e.g., a 384-well plate), automated liquid handling systems can dramatically increase the efficiency of processing. In some cases, a liquid handling system can comprise a temperature-controlled chamber and/or deck for controlling the temperature of molecule temperatures during transfer or reagent addition. In some cases, an automated liquid handling system can be used to maintain one or more nucleic acids at a temperature of from 0° C. to 10° C., 0° C. to 8° C., 0° C. to 6° C., 0° C. to 5° C., 0° C. to 4° C., 2° C. to 6° C., 3° C. to 5° C., 3° C. to 4° C., or 4° C. to 5° C. In some cases, a sample collection device can be used to maintain a sample collected from a subject (e.g., including one or more nucleic acids within the sample) within 0.1° C., within 0.5° C., within 1.0° C., within 1.5° C., within 2.0° C., within 2.5° C. or within 5.0° C. of a target temperature. In some cases, a target temperature is from 2° C. to 6° C. In some cases, a target temperature is 4° C.

### Sample Collection Devices

A system disclosed herein can comprise a sample collection device 102, 202. In some cases, a sample collection device 102, 202 can comprise a swab (e.g., a nasopharyngeal swab or a buccal swab), a vessel (e.g., a cup, vial, sample tube, etc.), or an absorbent material (e.g., a dried blood sample collection card, which can comprise a filter paper). In some cases, a sample collection device 102, 202 can be temperature-controlled (e.g., refrigerated). In some cases, a sample collection device 102, 202 can comprise a refrigeration unit. In some cases, a sample collection device 102, 202 can comprise a temperature tracker (e.g., an analog or digital thermometer, which may comprise an alarm). In some cases, a sample collection device 102, 202 can comprise a refrigerated container (e.g., a box, cabinet, chest, or compartment of a vehicle) into which a swab, vial or tube can be placed for storage and/or transport. Controlling the temperature of samples (e.g., from the time of collection to the time of nucleic acid isolation or from the time of collection to the time of hitpicking) can greatly increase the quality (e.g., integrity) of nucleic acid samples isolated from the sample, which can, in turn, increase the accuracy and/or decrease sample-to-sample variability in PCR and/or molecular sequencing assays. In some cases, a sample collection device can be used to maintain a sample collected from a subject (e.g., including one or more nucleic acids within the sample) at a temperature of from 0° C. to −110° C., −10° C. to −90° C., −20° C. to −80° C., −70° C. to −90° C., −75° C. to −85° C., −15° C. to −35° C., −15° C. to −20° C., −20° C. to −25° C., or −18° C. to −22° C. In some cases, a sample collection device can be used to maintain a sample collected from a subject (e.g., including one or more nucleic acids within the sample) within 0.1° C., within 0.5° C., within 1.0° C., within 1.5° C., within 2.0° C., within 2.5° C. or within 5.0° C. of a target temperature. In some cases, a target temperature is from −18° C. to −22° C. In some cases, a target temperature is −20° C. In some cases, a target temperature is from −75° C. to −85° C. In some cases, a target temperature is −80° C. In some cases, a sample collection device can be incorporated or adapted for use in a STAT protocol, which can comprise urgent analysis of samples obtained from subjects in a hospital setting, e.g., as described herein.

### Isolation Apparatuses

In some cases, a system disclosed herein can comprise a nucleic acid isolation apparatus 104, 204, e.g., for extraction and/or purification of nucleic acid molecules (e.g., RNA molecules) from a sample obtained from a subject. In some cases, a nucleic acid isolation apparatus 104, 204 can comprise one or more nucleic acid isolation reagents. In some cases, a nucleic acid isolation reagent can include a cell lysis reagent, a nucleic acid denaturant, and/or an RNA stabilization solution (e.g., RNAlater™ from Invitrogen™). In some cases, a nucleic acid isolation apparatus 104, 204 can comprise a centrifuge (e.g., a refrigerated centrifuge). In some cases, a nucleic acid isolation apparatus 104, 204 can comprise a filtration membrane. In some cases, a nucleic acid isolation apparatus 104, 204 can comprise a nucleic acid collection vessel (e.g., a tube or vial). In some cases, a nucleic acid isolation apparatus 104, 204 can comprise an ultraviolet (UV) spectrometer, e.g., for determining a concentration and/or purity of isolated nucleic acid molecules. For example, a nucleic acid isolation apparatus or portion thereof (e.g., a UV spectrometer of a nucleic acid isolation apparatus) can be used to determine a light absorbance ratio (e.g., a 260 nanometer (nm) wavelength light absorbance to 230 nm wavelength light absorbance (e.g., an “A260/230” ratio, or a “260/230” ratio) or a 260 nm wavelength light absorbance to 280 nm wavelength light absorbance ratio (e.g., an “A260/280” ratio or a “260/280”) ratio for the a plurality of nucleic acid molecules (e.g., isolated or extracted from a biological sample). In some cases, co-contaminates can be introduced during a processing of a plurality of nucleic acid molecules for high-throughput sequencing. In some cases, an A260/230 ratio can be useful in determining a level of contamination of a sample comprising nucleic acid molecules (e.g., wherein a contaminate may comprise an organic compound, such as Trizol, phenol, Guanidine HCl, and/or guanidine thiocyanate). In some cases, a sample comprising a plurality of nucleic acid molecules having a 260/230 ratio from 2.0 to 2.2 can be used for methods and systems described herein. In some cases, a sample comprising a plurality of nucleic acid molecules having a 260/230 ratio between 2.2 to 2.3, from 2.3 to 2.4, or from 2.4 to 2.5 can be useful for methods and systems described herein. In some cases, a sample comprising a plurality of nucleic acid molecules having a 260/230 ratio between 1.9 to 2.0, from 1.8 to 1.9, or from 1.7 to 1.8 can be useful for methods and systems described herein. In some cases, a nucleic acid isolation apparatus 104, 204 can comprise a sample purification system (e.g., Thermofisher® KingFisher™ Flex). In some cases, a nucleic acid isolation apparatus 104, 204 can comprise a bioanalyzer (e.g., an Agilent® 2100 Bioanalyzer™). In some cases, a bioanalyzer can be used to determine nucleic acid integrity, e.g., through the production and/or analysis of an electropherogram. In many cases, nucleic acid isolation (e.g., comprising the use of a nucleic acid isolation apparatus 104, 204) can improve nucleic acid stability and/or quality (e.g., because the nucleic acids are purified from enzymes in the sample that may degrade the nucleic acids). In some cases, PCR amplification and/or molecular sequencing can be more accurate and/or more repeatable if a nucleic acid isolation apparatus is used.

### Storage Apparatuses

A system disclosed herein can comprise a storage apparatus. In some cases, a storage apparatus can comprise a refrigerator. In some cases, a storage apparatus can comprise a container (e.g., a refrigerated container such as a container (e.g., a box, chest, or compartment of a vehicle) comprising a refrigeration unit). In some cases, a storage apparatus can comprise a refrigerant or a coolant (e.g., wet ice or dry ice). In some cases, a storage apparatus can be used to transport a sample or nucleic acid (e.g., a plurality of RNA molecules following RNA isolation from a sample, for instance as shown in FIG. 1A at 302). In some cases, a storage apparatus can be used to maintain a sample collected from a subject and/or one or more nucleic acids of the sample at a temperature of from 0° C. to −110° C., −10° C. to −90° C., −20° C. to −80° C., −70° C. to −90° C., −75° C. to −85° C., −15° C. to −35° C., −15° C. to −20° C., −20° C. to −25° C., or −18° C. to −22° C. In some cases, a storage apparatus can be used to maintain a sample collected from a subject and/or one or more nucleic acids of the sample within 0.1° C., within 0.5° C., within 1.0° C., within 1.5° C., within 2.0° C., within 2.5° C. or within 5.0° C. of a target temperature. In some cases, a target temperature is from −18° C. to −22° C. In some cases, a target temperature is −20° C. In some cases, a target temperature is from −75° C. to −85° C. In some cases, a target temperature is −80° C.

In some aspects, the system as disclosed herein comprises a series of arrays. In some cases, the series of arrays comprises at least a first array and a second array. In some cases, the array is a duplicate array. The duplicate array, and samples therein, undergo a qPCR step, wherein the sequencer communicates with the PCR analysis to only transfer positive samples onto the sequencing plate. In some aspects, the method comprises automatically formatting the sequencing plate to identify and select only positive samples or positive sample groups from the qPCR system. In some cases, the array comprises a 384-well plate. Arrays may be chosen in vertical arrays of 16 or horizontal arrays of 24, allowing for parallel pipetting.

### Database

In some aspects, the system as disclosed herein can comprise a database. In some embodiments, a database can be a part of a computer system described herein. In some embodiments, any data generated by any methods described herein can be saved or stored in a database. For example, Ct values generated by RT-PCR or data generated by analysis modules described herein can be saved or stored in a database. In some embodiments, data stored in a database can be communicated. For example, Ct values stored in a database can be communicated to a processing device or an analysis module. In some embodiments, data communication can be achieved through an indicated communication medium to a server at a local or a remote location. A communication medium can include any means of transmitting and/or receiving data. For example, the communication medium can be a network connection, a wireless connection, an intranet connection, or an internet connection. Such a connection can provide for communication over the World Wide Web. It is envisioned that data relating to the present disclosure can be transmitted over such networks or connections for reception and/or review by a party.

In some embodiments, the database described herein can be accessible through a communication medium described herein. In some embodiments, the database can be accessed by scanning a barcode (e.g., a skinny barcode, a fat barcode, or a QR code) on a container comprising a sample. For example, a container (e.g., a tube, a vial, or a plate) comprising a biological sample or RNA extracted from a biological sample can have a barcode on the container that can be scanned by a technician to retrieve data for that biological sample or RNA extracted from the biological sample from an online database. In some embodiments, data can be transmitted to a computer system or a mobile device by scanning a barcode on a container comprising a biological sample or RNA extracted from a biological sample. In some embodiments, the database can be accessed using a computer system, for example, a computer system with a network connection or an internet connection. In some embodiments, the database can be accessed using a mobile device, for example, a mobile device with a network connection, a wireless connection, an intranet connection, or an internet connection. In some embodiments, a barcode can be used to retrieve the data stored for a sample to track the status of the sample.

### Methods

One or more samples obtained (e.g., directly or indirectly) from one or more respective subjects can be tested via RNA extraction and RT-qPCR-based testing at a second location (e.g., wherein the second location is remote from a first location at which the extracted RNA can be sequenced). After categorizing samples as either PCR-positive (e.g., wherein the Ct value is below or no more than about 30, 31, 32, 33, 34, 35, 36, or 37) or negative (e.g., wherein the Ct value is above or higher than about 30, 31, 32, 33, 34, 35, 36, or 37), 96-well RNA plates of samples may be subsequently transported to the first location 100. Samples which are positive for at least a portion of a viral genome (or, in some cases, a bacterial genome) can then be automatically reformatted, or hit-picked, into 384-well plates using an Opentrons OT-2 pipetting robot or Tecan. The positive samples can then be reverse-transcribed using tiled, multiplexed primers (for e.g., ARTIC V3), tagmentation-based library prep and barcoding can be performed on the resulting cDNA, and samples can be pooled and sequenced on an appropriate sequencer (for e.g., MiSeq or NextSeq) for the volume of samples being processed at that time.

In some cases, the sample can be obtained from a subject via a collection device (e.g., a nasopharyngeal swab or buccal swab) at a second location, which may be local or remote relative to a first location (e.g., at which sequencing of extracted nucleic acids can be performed). In addition to the second location where swab samples are obtained, some example embodiments may utilize a parallel workflow to directly accept swabs from the first location, for genome sequencing. In some cases, the first location can be local to or adjacent to the second location. In some cases, the second location can be remote or distant relative to the first location. In some cases, the first location can comprise one or more of a hospital emergency center, an urgent care clinic, a community clinic, a primary care office, a nursing office, or a combination thereof. The swabs from the first location may be diagnosed as positive for COVID-19 by the local platform at the first location. In some cases, and as seen in FIG. 1A, the RNA can then be extracted from the samples at the first location.

In some cases, the method as disclosed herein comprises a series of arrays. In some cases, the series of arrays comprises at least a first array and a second array. In some cases, the array is a duplicate array. The duplicate array, and samples therein, undergo a qPCR step, wherein the sequencer communicates with the PCR analysis to only transfer positive samples onto the sequencing plate. In some cases, the method comprises automatically formatting the sequencing plate to identify and select only positive samples or positive sample groups from the qPCR system. In some cases, the array comprises a 384-well plate. Arrays may be chosen in vertical arrays of 16 or horizontal arrays of 24, allowing for parallel pipetting.

In some cases, the method comprises hitpicking. In some embodiments, hitpicking is automated. Automation enables screening methods to be performed in a high-throughput fashion. In some cases, the method disclosed herein enables positive samples to be sequenced at a higher stringency or sent to a facility for further processing. In some cases, following separation of positive sample groups from negative sample groups, the method comprises another array of positive samples ready for downstream processing and sequencing.

In some cases, a method can comprise sequencing one or more pluralities of nucleic acid molecule populations. In some cases, a method can comprise sequencing one or more subsets of one or more respective pluralities of nucleic acid molecules. In some cases, determining whether or not a subject has an infectious agent (e.g., determining that a subject with an unknown risk, a high risk, or a low risk of having an infectious agent) can comprise sequencing a nucleic acid molecule population derived from a sample obtained (e.g., directly or indirectly) from a subject and/or producing one or more alignments of a nucleic acid population versus a reference genome or portion thereof (e.g., using an analysis module comprising a sequencing device, such as a molecular sequencer).

In some cases, a method described herein can comprise sequencing one or more nucleic acid populations (e.g., one or more cDNA populations of a subset of a plurality of nucleic acid populations identified as hit(s) by amplification analysis), for instance to determine a nucleotide sequence of the one or more nucleic acid populations. In some cases, determining a nucleotide sequence of the one or more nucleic acid populations can comprise determining that a nucleotide sequence corresponding to a variant of concern (VOC) of an infectious agent (e.g., a virus or other contagious pathogen, such as a bacterium) is present in the nucleic acid population. In some cases, determining a nucleotide sequence of the one or more nucleic acid populations can comprise determining that a nucleotide sequence corresponding to a novel variant of concern (VOC) of an infectious agent (e.g., a virus or other contagious pathogen, such as a bacterium) is present in the nucleic acid population. In some cases, determining a nucleotide sequence of the one or more nucleic acid populations can comprise determining that a subject has an infectious agent (e.g., a virus or other contagious pathogen, such as a bacterium). In some cases, determining a nucleotide sequence of one or more nucleic acid populations corresponding to sample(s) obtained from one or more subjects can be used to determine an extent to which an infectious agent has spread within the population. In some cases, determining a nucleotide sequence of one or more nucleic acid populations corresponding to sample(s) obtained from one or more subjects can be used to determine a rate of mutation of an infectious agent (e.g., a virus or other contagious pathogen, such as a bacterium), for instance, by determining a rate at which novel variants of concern are identified within a population of subjects. In some cases, determining a nucleotide sequence of one or more nucleic acid populations corresponding to sample(s) obtained from one or more subjects can be used to determine a rate of transmission of an infectious agent (e.g., a virus or contagious pathogen, such as a bacterium) within a population of subjects, for instance, by determining a rate at which subjects within the population are identified as having the infectious agent.

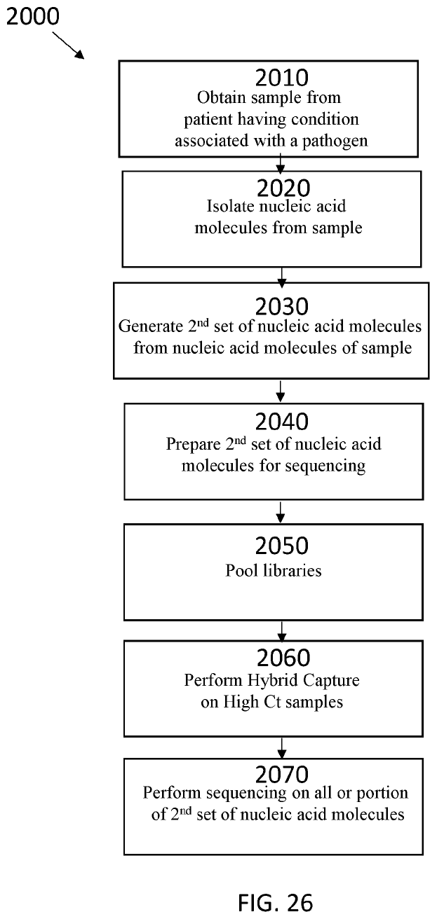

Turning to FIG. 26, a method 2000 can comprise obtaining a sample (e.g., via a buccal or nasal swab, or a urine sample) comprising a condition associated with a pathogen (step 2010), isolating or extracting a nucleic acid molecule (e.g., RNA) from the sample (step 2020), generating a second set of nucleic acid molecules (e.g., cDNA) (step 2030), preparing the second set of nucleic acid molecules for sequencing (e.g., via tagmentation or barcoding, as described herein) (step 2040), pooling two or more sequencing libraries (step 2050), performing hybrid capture on the pooled libraries (for instance, on a portion of the pooled libraries having a Ct value) (step 2060), and performing sequencing on all or a portion of the second set of nucleic acid molecules.

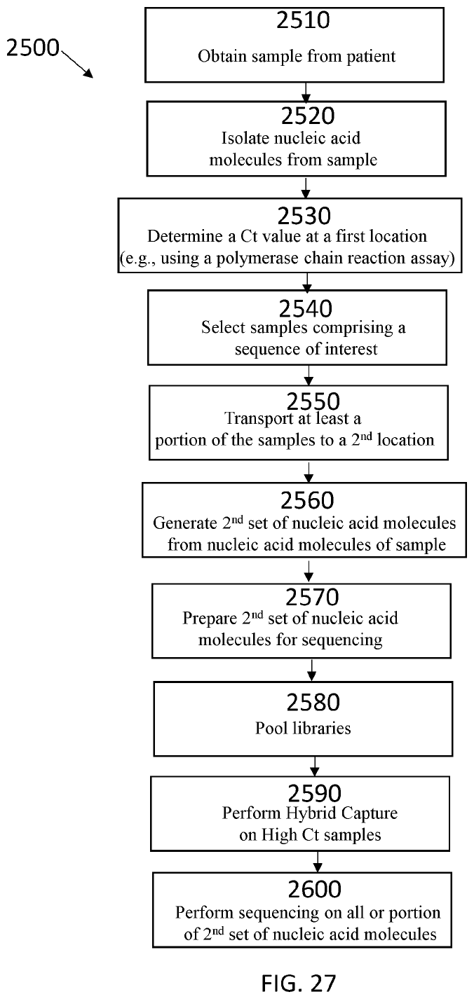

As seen in FIG. 27, a method 2500 can comprise obtaining a sample (e.g., via a buccal or nasal swab, a urine sample, or a surface swipe test) (step 2510), isolating or extracting a nucleic acid molecule (e.g., RNA) from the sample (step 2520), determining a Ct value (e.g., using a polymerase chain reaction assay, such as qPCR) (step 2530), selecting samples comprising a sequence of interest (e.g., based on the Ct value) (step 2540), transporting at least a portion of the samples to a second location (step 2550), generating a second set of nucleic acid molecules from the nucleic acid molecules of the sample (step 2560), preparing the second set of nucleic acid molecules for sequencing (step (2570), pooling two or more sequencing libraries (step 2080), performing hybrid capture on high Ct value samples (step 2590), and performing sequencing on all or a portion of the second set of nucleic acid molecules (step 2600).

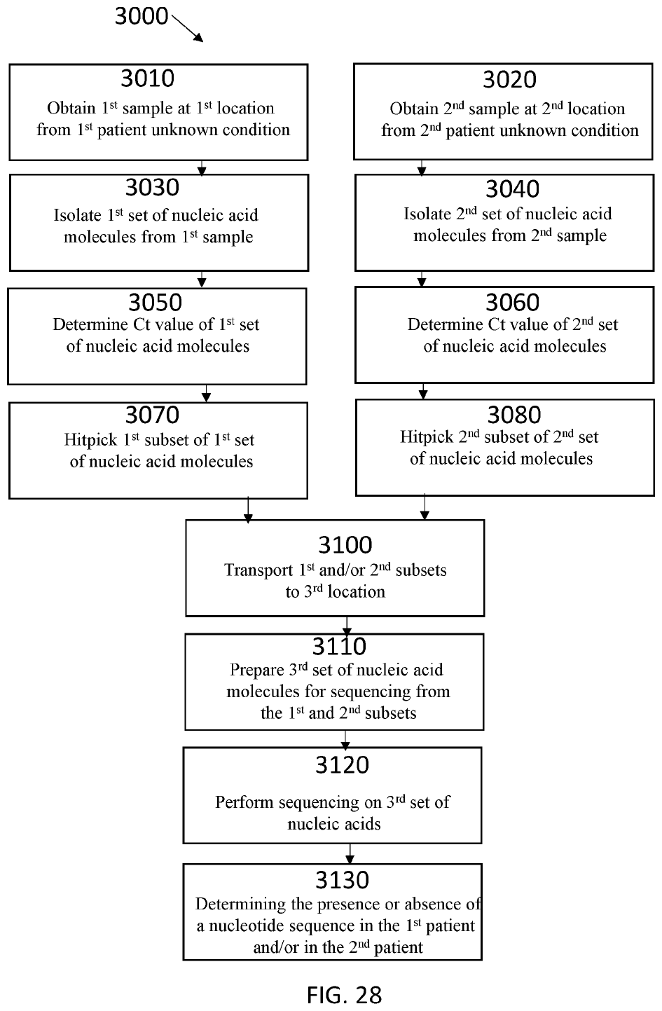

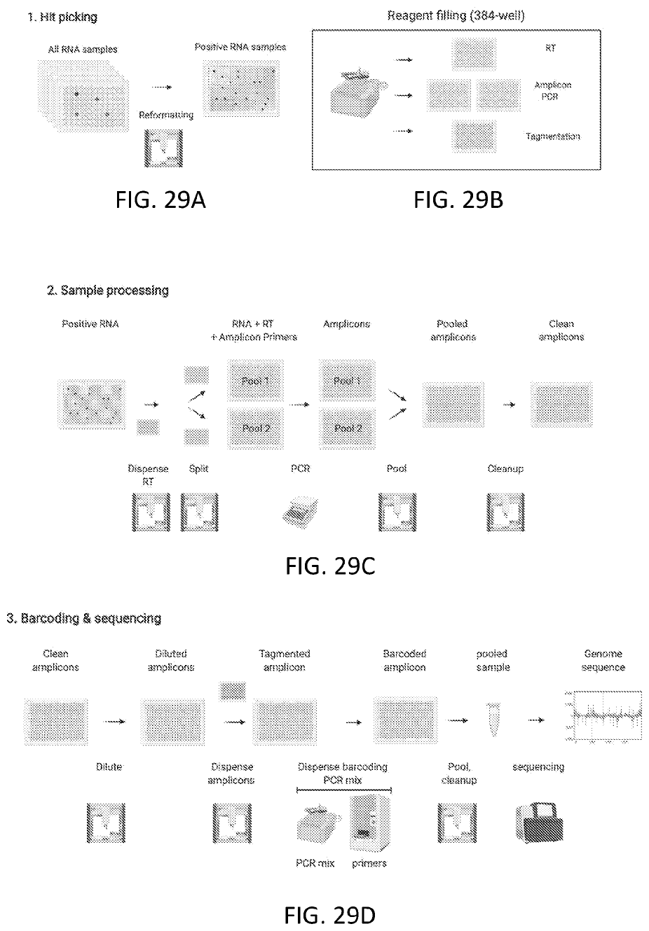

FIG. 28 shows a method 3000 comprising step 3010 for obtaining a first sample at a first location from a first patient having an unknown status with respect to a condition caused by a pathogen of interest, step 3030 isolating a first set of nucleic acid molecules from the first sample, step 3050 determining a Ct value of the first set of nucleic acid molecules, step 3070 hitpicking a first subset of the first set of nucleic acid molecules, step 3020 obtaining a second sample at a second location from a second patient having an unknown status with respect to a condition caused by a pathogen of interest, step 3040 isolating a second set of nucleic acid molecules from the second sample, step 3060 determining a Ct value of the second set of nucleic acid molecules, step 3080 hitpicking a second subset of a second set of nucleic acid molecules, step 3100 transporting the first and/or second subsets to a third location, step 3110 preparing a third set of nucleic acid molecules for sequencing from the first and second subsets, step 3120 performing sequencing on the third set of nucleic acid molecules, and step 3130 determining the presence or absence of a nucleotide in the first patient and/or in the second patient.

### Nucleic Acid Amplification Analysis

Nucleic acid amplification analysis (e.g., comprising one or more of polymerase chain reaction (PCR), quantitative or real-time PCR (qPCR), reverse transcription-PCR (RT-PCR), quantitative or real-time RT-PCR (“rRT-PCR” or “RT-qPCR”), and/or isothermal amplification analysis) can be useful in system, devices, and methods described herein. For example, nucleic acid amplification can provide a value (e.g., amplification cycle threshold (Ct) value, which can indicate the presence or absence of a nucleotide sequence of interest in a nucleic acid population derived from a sample) upon which subsequent handling, processing (e.g., comprising hitpicking, which may be automatically performed based on the value), and/or sequencing may be entirely or partially based. In some cases, certain compositions (e.g., reagents), methods, kits, apparatuses (e.g., analysis modules), and/or apparatus configurations may be employed to advantageous improve a system or method described herein.

### Sample Processing

### RNA Transport, Storage, and Hitpicking

Preservation of RNA sample quality before library preparation for sequencing is the crucial first step of the sequencing pipeline. We observed that RNA degradation caused by even short times at ambient temperature or freeze-thaw cycles, as may occur during transport, storage, or hitpicking, dramatically reduces the reconstruction success rate for SARS-CoV-2 positive samples and can be detected as a shift in Ct value for these samples. This is true especially for samples with low RNA concentration (high Ct). To avoid these issues, we implemented a strictly regimented process for temperature control during shipping, storage, and hitpicking to preserve RNA quality between the diagnostic lab and sequencing lab facilities. To confirm that these methods preserve RNA quality, RT-qPCR using both the CDC N1 and N2 primer-probe sets is regularly performed after hitpicking at our sequencing facility and compared to those reported at the PRL testing facility. Ct values for both primer-probe sets generally align closely with the original values obtained at the Manhattan testing facility, indicating that these quality control measures are successful in preserving RNA quality for downstream sequencing, and any deviation from expected Ct values are quickly observed and corrective measures implemented.

Following testing, RNA plates containing both negative and positive samples can be stored at approximately −20° C. until transport for sequencing. Sample plates can be shipped each morning on dry ice, and temperature trackers can be used to ensure maintenance of temperatures below approximately −20° C. Following transport, the samples can be immediately accessed, sorted, and stored at approximately −80° C. to await hitpicking. Samples can be thawed, and positive samples are hitpicked into a new 384-well plate using an Opentrons OT-2 robot or Tecan equipped with a temperature deck, e.g., to ensure that samples are kept at a constant approximate −4° C. RT-qPCR using both the N1 and N2 primer-probe sets can be performed after hitpicking (e.g., to confirm preserved RNA quality) and the values obtained can be compared to those reported at the second location 200. Plates containing samples comprising a sequence of interest (e.g., nucleic acid populations having a Ct value below a threshold value) can be either sequenced using an analysis module comprising a sequencing device or stored at −80° C. prior to RT-PCR.

RNA degradation prior to RT-PCR, such as during transport, storage, or hitpicking, can dramatically reduce the reconstruction success rate for positive samples, especially for samples with low RNA concentration (high Ct). To reduce or eliminate the impact of such issues, means for controlling temperature during shipping, storage, and hitpicking preserves RNA quality can be provided. In many cases, Ct values for both primer-probe sets can align closely with the original values obtained at the testing facility, which can indicate that these quality control measures can preserve RNA quality for downstream sequencing.

Reformatting of Positive Samples into a Single Plate (Hitpicking)

In some cases, 96-well plates containing a random assortment of samples which tested positive or negative, for SARS-CoV-2 RNA in some example embodiments, can be shipped on dry ice from a testing facility (e.g., at a second location) to a research and development facility (e.g., at a first location). In some cases, a custom hitpicking script or a computer processor program can be generated for an OT-2 robot or Tecan to reformat or rearrange all positive samples into a single 384-well plate starting with the lowest Ct sample (e.g., in well A1) and ascending row by row. In some cases, a positive control can be added (e.g., at wells B2 and P22), and a negative control (e.g., in well P24), for example such that quadrant 4 of a given plate could be tested for quality control and contain both positive controls and the negative control, along with test samples of increasing Ct.

### RNA Quality Control

Preservation of RNA sample quality before library preparation for sequencing can be an important first step of the sequencing system. RNA degradation prior to RT-PCR, such as during transport, storage, or hitpicking, dramatically can reduce the reconstruction success rate for SARS-CoV-2 positive samples, especially for samples with low RNA concentration (high Ct). Some example embodiments can implement a process for temperature control during shipping, storage, and hitpicking to preserve RNA quality. RT-qPCR using both the N1 and N2 primer-probe sets can be performed regularly after hitpicking and compared to those reported at the testing facility (e.g., at the second location), e.g., to confirm that these methods preserved RNA quality. In many cases, Ct values for both primer-probe sets can align closely with the original values obtained at the testing facility at the second location, which can indicate that these quality control measures are successful in preserving RNA quality for downstream sequencing.

### Nucleic Acid Sequencing Analysis

### Barcoding of Tagmented DNA and Library Amplification

In some cases, barcoding MasterMix can be prepared by mixing approximately 1-10 μL of 2× Kapa Hifi HotStart ReadyMix with approximately 1-10 μL of nuclease-free water for each well. Following tagmentation, Kapa Mix can be dispensed directly onto the tagmentation plate using MANTIS with high-volume chips. In some cases, approximately 0.125 μL of 100 μM of a first sequencing adapter (e.g., 100 μM of a 5′ Illumina barcoding adapter, for example, n7xx wherein “xx” is a numerical label of the barcoding adapter) and approximately 0.125 μL of 100 μM of a second sequencing adapter (e.g., 100 μM of a 3′ Illumina barcoding adapter, for example, n5xx wherein “xx” is a numerical label of the barcoding adapter) can be dispensed consecutively onto the sample plate using an acoustic dispenser. Following addition of the primers, the barcoded samples can be placed on a thermocycler for a barcoding PCR reaction. The optimized barcoding PCR reactions can include the following steps: approximately 72° C. for 5 minutes, approximately 98° C. for 5 minutes, approximately 13 cycles of approximately 10 seconds of denaturation at 98° C., 30 sec of annealing at 66° C., and 30 seconds of extension at 72° C. A final extension is performed for approximately 5 min at 72° C. before cooling to approximately 10° C.

### Tagmentation and Barcoding

In some embodiments, tagmentation and barcoding add sequencing adapters (e.g., from Illumina) and index sequences to the RT-PCR product, for instance, so that multiple samples can be pooled and sequenced together. The input DNA and Nextera tagmentation enzyme ratio can determine the library size. In some cases, different amounts of amplicon input and different tagmentation enzyme concentration can be used, e.g., to achieve optimal size distribution.

Library Pooling and Loading onto Illumina Sequencer

In some embodiments, 2 μL of each DNA library can be pooled into a single tube before column purification. In some embodiments, column purification can be performed using DNA Clean & Concentrator-5 (Zymo Research). The pooled libraries can be eluted in 50 μL nuclease-free water. DNA concentration of the sample can be measured using Qubit 1× dsDNA HS Assay Kit and the Plate reader (BioTech Synergy H1), and the sample can be further diluted to approximately 4 nM. The library can be denatured and diluted according to the Denature and Dilute Libraries Guide by Illumina for MiSeq and NextSeq, before being loaded on the Illumina sequencers.

### Sequencing Production Line Samples

Approximately 100-50,000 patient samples can be tested for COVID-19 every day at the second location 200, for instance, using a qPCR-based method (see, U.S. patent application Ser. No. 17/478,415, which is incorporated herein by reference for all purposes). The extracted RNA plates used for testing can be stored at approximately −20° C. freezer immediately after testing, for instance, until they are delivered to a facility for sequencing. Positive samples (e.g., nucleic acid populations having a Ct value below a hit threshold) can be reformatted into a new 384-well destination plate via automated hitpicking (e.g., wherein samples having a Ct value below a hit threshold are automatically transferred into a new container while samples having a Ct value at or above the hit threshold are not transferred) using an Opentrons OT-2 or Tecan processing device. In some embodiments, a positive sample can comprise at least a portion of a viral genome (or, in some embodiments, a bacterial genome). In some embodiments, the viral genome (or bacterial genome) can comprise a SARS-CoV-2 genome. The positive RNA samples can be transferred into two plates containing RT-PCR master mix to allow separate reactions with Primer Pool 1 (P1) and Primer Pool 2 (P2). P1 and P2 plates undergo RT-PCR overnight and can be pooled together into a single plate diluting the samples to approximately 0.5 ng/uL. The samples can be tagmented using Illumina Tagment Mix and barcoded. The barcoded samples can be pooled together into a single tube, column purified, diluted, and denatured before being loaded onto the Illumina sequencer.

### Sequencing STAT Samples

A separate system and/or method for processing STAT samples can be implemented (e.g., in parallel to a production line system and/or method), e.g., in order to process high priority patient swabs sent directly from hospital emergency departments. The STAT samples can be accessioned, reformatted into 96-well plate, and extracted. In some cases, the STAT samples can be extracted via Kingfisher™ Flex (Thermo Scientific). Extracted RNA samples can be reformatted again into a 384-well plate using Cybio Felix (Analytik Jena), and the rest of the sequencing process can resume following the protocol described for samples from the second location 200.

### Genome Assembly

In some embodiments, sequencing adapters can be first trimmed, then aligned to a reference genome using. Reads that are unmapped, or reads that have secondary alignments, can be discarded from the alignment. A consensus genome and mutations from the wild-type sequence can be identified using samtools and Intrahost variant analysis of replicates (e.g., as described in Heng, Bioinformatics 2011 Nov. 1; 27(21):2987-2993 and at http://samtools.sourceforge.net and in Grubaugh et al Genome Biol. 2019; 20:8 and at github.com/Andersen-lab/ivar, which are each hereby incorporated by reference for all purposes) of replicates (iVar) with a minimum quality score of 20, frequency threshold of 0.6 and a minimum read depth of 10× coverage. In some cases, a consensus genome with ≥90% breath-of-coverage with ≤3000 ambiguous bases can be considered a successful reconstruction. In some cases, variants can be identified via using PANGOLIN v2.1.11 to v2.3.8.

### Pathogens and Infectious Diseases

In some cases, a system or method described herein can utilize an automated testing system, which can be capable of processing approximately 45,000 tests per day with a turnaround time of approximately 24 to 48 hours, in some cases. The increased capacity of systems and methods described herein can be critical to a major metropolitan area's ability to meet the testing needs brought on by a severe and/or widespread infectious disease event, which may be characterized by multiple successive waves of increased infections among a population, for example, due to the emergence of new variants of the infectious agent.

In some cases, a system or method described herein can be used to identify or monitor a pathogen (e.g., a variant of concern of a pathogen) in a population. In some cases, the pathogen (e.g., a variant of concern of the pathogen) can be a virus or a viral variant. For example, the pathogen can comprise or be associated with a human disease-causing RNA virus. In some cases, the pathogen (e.g., a variant of concern of the pathogen) can be a bacterium or strain of a bacterium, such as Clostridioides difficile. In some cases, a pathogen (or disease associated with a pathogen) that can be involved in an aspect of the present disclosure (e.g., a condition of a subject or patient, a sequence or portion thereof shared with a nucleic acid, presence within a sample or specimen, a condition or variant of concern monitored, tracked, or surveilled by a system or method described herein) can include orthomyxoviruses, Hepatitis C Virus (HCV), Ebola disease (e.g., Ebola Hemorrhagic Fever), SARS, MERS (Middle Eastern Respiratory syndrome, MERS-CoV), influenza (e.g., influenza type A or influenza type B), polio, measles, retroviruses, Human T-Cell Lymphotropic virus type 1 (HTLV-1), sexually transmitted diseases, hepatitis A, hepatitis B, hepatitis D, hepatitis E, chlamydia, herpes, Herpes Zoster, Creutzfeldt-Jacob Disease, Dengue fever, babesiosis, anthrax, yellow fever, Rift Valley fever, Zika virus, Nipah, Crimean-Congo haemorrhagic fever, monkeypox, smallpox Marburg virus disease, cholera, polio, lassa fever, meningitis, norovirus, HFMD (hand, foot, and mouth disease), pertus diptheria, tuberculosis, encephalitis (e.g., arboviral), human immunodeficiency virus (HIV), leptospirosis, Clostridioides difficile, Staphylococcus aureus, Yersinia pestis, methicillin-resistant Staphylococcus aureus, impetigo, or salmonella.

In some cases, significant variants of concern (VOCs) of the SARS-CoV-2 pandemic can include Alpha (B.1.1.7), Beta (B.1.351), Gamma (P.1), Delta (B.1.617.2), and Omicron (B.1.1.529), e.g., due to their dominant representation in the community or their comparatively poorer clinical prognosis. Delta (B.1.617.2) and Omicron (B.1.1.529) can be of particular concern over their high transmissibilities and potential for “breakthrough” cases in fully vaccinated individuals. Accordingly, a widespread genomic biosurveillance system can be desirable for tracking the emergence and spread of VOCs, which can allow for more informed policy decisions and more effective public health countermeasures.

### Samples

In some cases, systems, methods, kits, and devices disclosed herein can be utilized to analyze a broad range of samples. In some cases, a sample can be a biological sample. In some cases, a sample can be derived from a biological sample (e.g., extracted from a biological sample). A sample can comprise a nucleic acid molecule. In some cases, a sample can comprise a nucleic acid molecule of interest (e.g., a nucleic acid molecule comprising a sequence of interest, which may comprise a variant of concern (VOC)). In some cases, a sample can comprise a ribonucleic acid (RNA) molecule or portion thereof. In some cases, a sample can comprise a deoxyribonucleic acid (DNA) molecule or portion thereof. In some cases, a sample can comprise cell-free DNA (e.g., circulating cell-free DNA, for example, as obtained from a blood sample or derivative thereof).

In some cases, a sample can comprise or be derived from a cell (e.g., a stem cell, a multipotent cell, a terminally differentiated cell, or an immortalized cell). In some cases, a sample (or specimen comprising a sample) can comprise or be derived from whole blood, blood plasma, blood serum, urine, cerebrospinal fluid (CSF), sputum, saliva, bone marrow, synovial fluid, aqueous humor, amniotic fluid, cerumen, breast milk, lavage (e.g., bronchoalveolar lavage fluid, auroral pharyngeal lavage fluid, or lavage fluid from sinus cavities), nasopharyngeal fluid, semen, prostatic fluid, Cowper's fluid, pre-ejaculatory fluid, female ejaculate, sweat, tears, ear exudate, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, bronchopulmonary aspirate, blastocoel cavity fluid, or umbilical cord blood. A sample can be obtained or derived from an animal subject (e.g., wherein the animal subject is a human, a mouse, a rat, a dog, a cat, a horse, a bat, a cow, a monkey, a bat, a rabbit, a hamster, a gerbil, a goat, a sheep, a pig, a raccoon, a squirrel, an insect (e.g., a mosquito or a fly), a bird (e.g., a pigeon or a chicken).

In some cases, a sample can be obtained or derived from a subject (e.g., an animal subject) known to have a disease or condition (e.g., a transmissible condition, such as an infectious disease). In some cases, a sample can be obtained directly from a subject (e.g., an animal subject) known to have a disease or condition (e.g., a transmissible condition, such as an infectious disease). For example, a specimen comprising a sample (e.g., a specimen comprising a nucleic acid molecule or portion thereof) can be collected from a subject, for instance by a doctor or laboratory technician. For example, a sample (or specimen comprising the sample) can be collected by a non-invasive means (e.g., a nasopharyngeal swab, a nasal swab, an oropharyngeal swab, a buccal swab, a saliva sample, a urine sample, or a stool sample). A biological sample can be collected by the subject providing the sample to, for example, a doctor or lab technician. For example, the subject can provide a urine, stool, or saliva sample. In some cases, a sample (or a specimen comprising the sample) can be collected by an invasive means. In some cases, a sample (or a specimen comprising the sample) can be collected by a minimally invasive means (e.g., drawing blood or another bodily fluid from the subject, for example, using a needle biopsy or fine needle aspiration technique). In some cases, collection of a sample (or a specimen comprising a sample) can comprise collection of a solid tissue from a subject, for example, via tissue biopsy (e.g., skin biopsy). In some cases, a specimen comprising a sample (e.g., a specimen comprising a nucleic acid molecule or portion thereof) can be collected by a subject and transferred to a sample analysis facility or site. In some cases, a sample can be obtained or derived from a subject (e.g., an animal subject) suspected of having a disease or condition (e.g., a transmissible condition, such as an infectious disease). In some cases, a sample can be obtained directly from a subject. In some cases, a human subject can be a patient (e.g., admitted to or treated at a health care facility).

In some cases, a sample can be obtained indirectly from a subject. For example, a sample can be obtained from a surface of an object touched by a subject or touched by a fluid or aerosol produced by the subject (e.g., via coughing, sneezing, or transfer of a bodily fluid to a hand or article of clothing and subsequent transfer to the object surface). In some cases, a sample can be obtained indirectly from a subject via a surface wipe test. In some cases, a sample can be obtained indirectly from a subject (or plurality of subjects) by testing a water (or other fluid) sample known or suspected to have come into contact with a sample from a subject. Increasing the type and frequency of sampling in a geographic region (e.g., comprising a subject population), such as collection and analysis of nucleic acids present in wastewater samples or obtained from environmental swabs, such as public transit swabs, can improve comprehensive biosurveillance for early detection of impending public health risks.

In some cases, a sample (e.g., a nucleic acid molecule or fragment thereof) can be obtained by extracting the sample from a biological specimen (e.g., a cell, biopsy, bodily fluid, or tissue sample) from a subject. In some aspects, one or more methods or method steps described herein can be performed on a sample (e.g., a nucleic acid molecule or plurality of nucleic acid molecules) extracted from a biological sample or specimen. For example, DNA (e.g., genomic DNA or cell-free DNA) or RNA can be extracted from one or more samples before analysis. In some cases, isolation or extraction of a nucleic acid molecule (e.g., RNA or DNA) from swabs, bodily fluids, or tissues can comprise disruption of a tissue or cell membrane, e.g., in the presence of protein denaturant(s), for example to quickly and effectively inactivate enzyme(s), such as RNAses, that may be present in the sample or environment in which the sample is extracted or isolated. Isolated total RNA can be further purified from protein contaminants of the sample or specimen and concentrated by selective ethanol precipitation, phenol/chloroform extractions followed by isopropanol precipitation or cesium chloride, lithium chloride or cesium trifluoroacetate gradient configurations.

In some cases, a sample (e.g., comprising a nucleic acid molecule or portion thereof) can be processed or analyzed. In some cases, a sample (e.g., a nucleic acid molecule or portion thereof) can be amplified, e.g., using a molecular biology technique. For instance, a sample (e.g., a nucleic acid molecule or portion thereof) can be assayed (e.g., processed or analyzed) using one or more of polymerase chain reaction (PCR), quantitative or real-time PCR (qPCR), reverse transcription-PCR (RT-PCR), quantitative or real-time RT-PCR (“rRT-PCR” or “RT-qPCR”), and/or isothermal amplification analysis, for example to amplify all or a portion (e.g., a sequence of interest, which may comprise a variant of concern) of the sample (e.g., the nucleic acid molecule or portion thereof). In some cases, a sample (e.g., a nucleic acid molecule or portion thereof or an amplification product (e.g., amplicon) of a nucleic acid molecule or portion thereof) can be sequenced, for instance to determine a nucleotide sequence of all or a portion of a nucleic acid molecule or portion thereof of the sample (e.g., to determine the presence or absence of a variant of concern in the nucleotide sequence).

In some cases, a nucleic acid molecule (e.g., RNA) can be transcribed into complementary DNA (cDNA), for example, following amplification. In some cases, cDNA can serve as a template for multiple rounds of amplification by an appropriate DNA polymerase. Reverse transcription reactions can be carried out using non-specific primers complementary to target RNA sequences for each probe being monitored, or using thermostable RNA-dependent DNA polymerases (such as AMV RT or a MMLV RT).

In some cases, a polymerase (e.g., for use in a PCR assay of a method or system described herein) can be selected to confer one or more advantages, including increased sequencing depth, increased success rate in the reconstruction of a genome or portion thereof (e.g., a genome or portion thereof of a pathogen of interest), or increased detection rate for a variant of concern. In some cases, a polymerase (e.g., a reverse transcriptase) with a moderate or high processivity (e.g., Taq polymerase or DNA polymerase III) can increase the speed and/or sequencing depth of a method or step of a method described herein. In some cases, a polymerase can be selected that has a processivity of at least about 40, at least about 50, at least about 60, or at least about 70 nucleotides per binding event. In some cases, a polymerase can be selected that has a processivity of at least about 500 nucleotides per second, at least about 750 nucleotides per second, or at least about 1000 nucleotides per second.

In some cases, an exonuclease with a processivity of at least about 30 nucleotides per second may be used. For example, an exonuclease may have a processivity of at least about 30, at least about 40, at least about 50, at least about 60, at least about 70, at least about 80, at least about 90, or at least about 100 nucleotides per second. In some embodiments, an exonuclease may have a processivity of at least about 60 nucleotides per second.

In some cases, a biological sample (e.g., comprising a nucleic acid molecule) can be subjected to one or more of a chemical treatment and/or a heat treatment. In some cases, a biological sample (e.g., comprising a nucleic acid molecule) can be treated with N-acetylcysteine (NAC) to aid in liquification of mucus that may be present in a specimen comprising the sample, such as a mucus specimen obtained directly or indirectly from a subject. In some cases, a biological sample (e.g., comprising a nucleic acid molecule) can be treated with NAC before the sample is extracted from the biological sample. In some cases, a biological sample (e.g., comprising a nucleic acid molecule) can be treated to inactivate an infectious agent or pathogen (e.g., viral, bacterial, fungal, or parasitic pathogen) before a sample is extracted. In some cases, a biological sample can be heat-inactivated before the sample is extracted from a specimen. In some cases, heat inactivation can be performed in a convection oven at 65° C. In some cases, a biological sample can be inactivated in a virus-inactivating buffer. In some cases, a biological sample can be inactivated using DNA/RNA Shield.

A sample can be a “STAT” sample. In some cases, a sample derived from a subject, which may comprise a nucleic acid molecule or portion thereof, can be processed and/or analyzed under urgent conditions (e.g., a “STAT” sample). In some cases, such a sample may be processed shortly after specimen acquisition, e.g., in cases where data from nucleic acid molecule analysis is urgently needed. For example, nucleic acid molecule analysis may be urgently needed for a sample obtained or derived from a patient received at an emergency department of a medical facility, for instance, to determine whether the patient may pose a risk of contagion to medical staff or other patients. In some cases, the requirements for storage and/or transportation may be different for a “STAT” sample compared to a non-STAT sample. For example, a “STAT” sample may be more likely to be obtained at a location (e.g., a medical facility, such as a hospital) that comprises resources for extraction, processing, amplification analysis, and/or sequencing analysis of one or more nucleic acid molecules in the sample. In some cases, a “STAT” sample may be obtained or derived from a subject having or suspected of having a high risk of an infectious agent (e.g., a virus or bacterium) and/or a high risk of a deleterious effect of having the infectious agent (e.g., wherein the subject presents with one or more measured values indicative of serious danger to health (e.g., low blood oxygenation and/or high temperature) and/or wherein the subject has multiple or serious comorbidities associated with severely negative outcomes of having the infectious agent).

A sample can be a “production line” sample. In some cases, a “production line” sample can be a non-STAT sample. In some cases, a “production line” sample may be a sample obtained as part of routine and/or baseline biosurveillance of a population of subjects. In some cases, a “production line” sample may be a sample obtained or derived from a subject with an unknown or low risk of having an infectious agent (e.g., a virus or bacterium) and/or a low risk of a deleterious effect of having the infectious agent (e.g., wherein the subject presents with few or no comorbidities associated with severely negative outcomes of having the infectious agent and/or wherein the subject presents with few or no measured values (e.g., comprising a temperature and/or blood oxygenation) indicative of serious danger to health). In some cases, a “production line” sample may be stored and transported as a batch with additional samples collected (e.g., and, optionally, extracted, processed, and/or analyzed, for instance using nucleic acid molecule amplification analysis) at a first location to a second location, for instance, where additional processing (e.g., hitpicking) and/or analysis (e.g., molecular sequencing) may be performed. In some cases, a “production line” sample can be obtained or derived from a subject with a significant (e.g., moderate or high) risk of having an infectious agent (e.g., a virus or bacterium) and/or a significant (e.g., moderate or high) risk of a deleterious effect of having the infectious agent. For example, a subject experiencing moderate or severe symptoms associated with a high risk of having an infectious agent and/or a high risk of having a deleterious effect of having the infectious agent may not have access (or convenient access) to a location equipped with advanced analysis equipment (e.g., an analysis module comprising a molecular sequencing apparatus and/or a processing device, which may be used in hitpicking and/or processing of a sample for nucleotide sequence analysis) and may provide a “production line” sample at a location at which initial processing and/or analysis of the sample (e.g., comprising nucleic acid molecule extraction and/or nucleic acid molecule amplification analysis). In some cases, a “production line” sample may require or benefit from implementation of one or more systems, devices, or method steps for improved storage and/or improved transportation, e.g., as described herein.

### Computer Systems

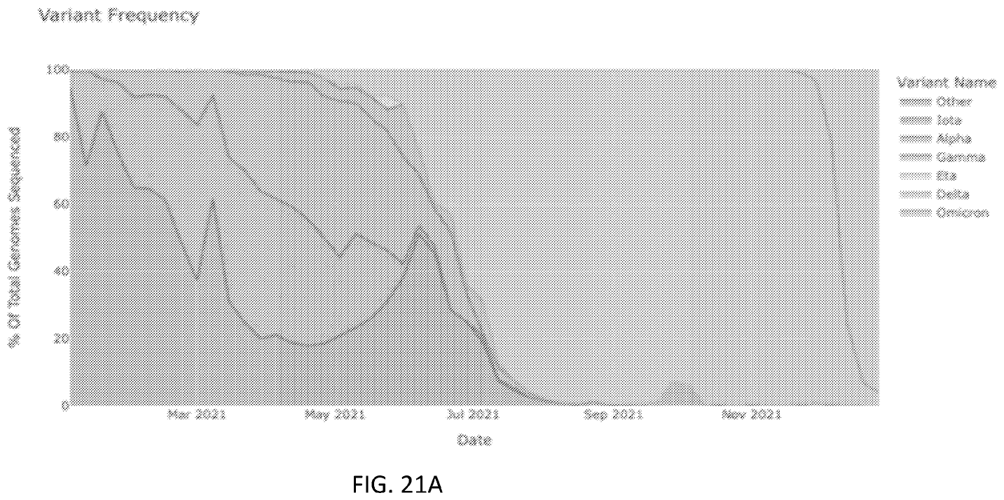

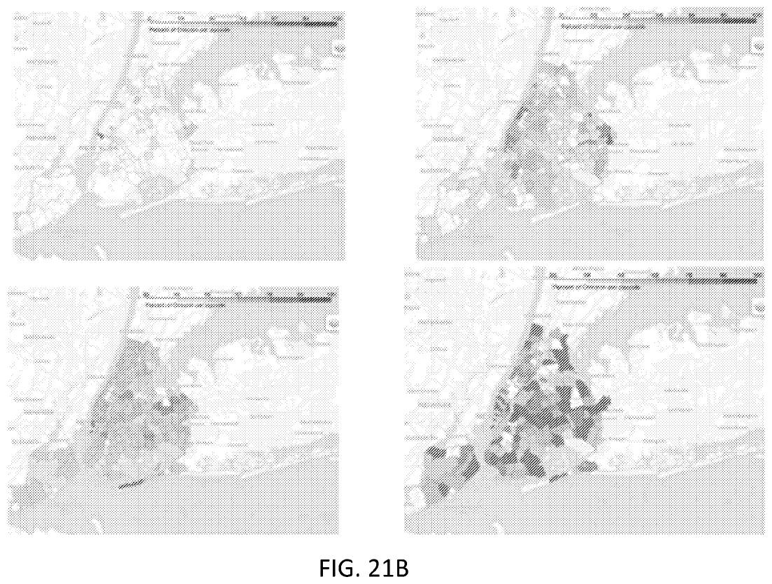

In some cases, a system, device, module, or method described herein can comprise a computer system. In some cases, a method or system described herein (e.g., implemented using a computer system) can be useful in developing, implementing, and/or evaluating a public health policy. For instance, the efficacy of a public health policy applied to a population can be evaluated, for instance, by determining a rate of infection with a pathogen of interest of individuals comprising the population and/or by determining a rate of emergence of novel variants of concern within the population. In some cases, a public health policy can be implemented using a method or system described herein. For example, a public health policy that comprises contingencies triggered by changes (e.g., real-time changes) in the spread of a pathogen in a population or the emergence of novel variants of concern within the population can be implemented by monitoring such metrics in the population using methods and systems described herein. In some cases, a public health policy can be modified or developed using data generated by methods or systems described herein, for instance, wherein the data comprises information regarding a mutation rate or transmissibility of a pathogen of interest (e.g., as ascertained by determining a rate of infection or spread of the pathogen among individuals in the population and/or by determining a rate of emergence of novel variants of concern in the population, which can in some cases be determined by a method or system comprising reconstruction of a genome of a pathogen). In some cases, a method or system described herein can comprise generation of a bioweather map, e.g., comprising geographical information regarding the location(s) at which patients positive for a pathogen of interest reside. For example, a histogram can be generated for the prevalence of one or more variants of concern within a population of subjects, e.g., over time (e.g., as shown in FIG. 21A). In some cases, a map can be generated from nucleic acid analysis described herein showing prevalence or progression of a pathogen within a geographical area at a given time or over a period of time (e.g., as shown in FIG. 21B). In some cases, a computer system can be used to determine a rate of mutation of an infectious agent (e.g., a virus or other contagious pathogen, such as a bacterium), for instance, by determining a rate at which novel variants of concern are identified within a population of subjects. In some cases, a computer system can be used to determine a rate of transmission of an infectious agent (e.g., a virus or contagious pathogen, such as a bacterium) within a population of subjects, for instance, by determining a nucleotide sequence of one or more nucleic acid populations corresponding to sample(s) obtained from one or more subjects to determine a rate at which subjects within the population are identified as having the infectious agent.

In some cases, a system, device, module, or method described herein can comprise a network of computer systems. In some cases, a system, module, or device described herein can be operably linked to a computer system. In some cases, the computer system can be local to the system, module, or device. In some cases, the computer system can be remote relative to the system, module, or device (e.g., wherein the computer system is connected by a wired or wireless connection to the system, module, or device). In some cases, a computer system can be configured to control, coordinate, and/or operate one or more systems, devices, and/or modules described herein.

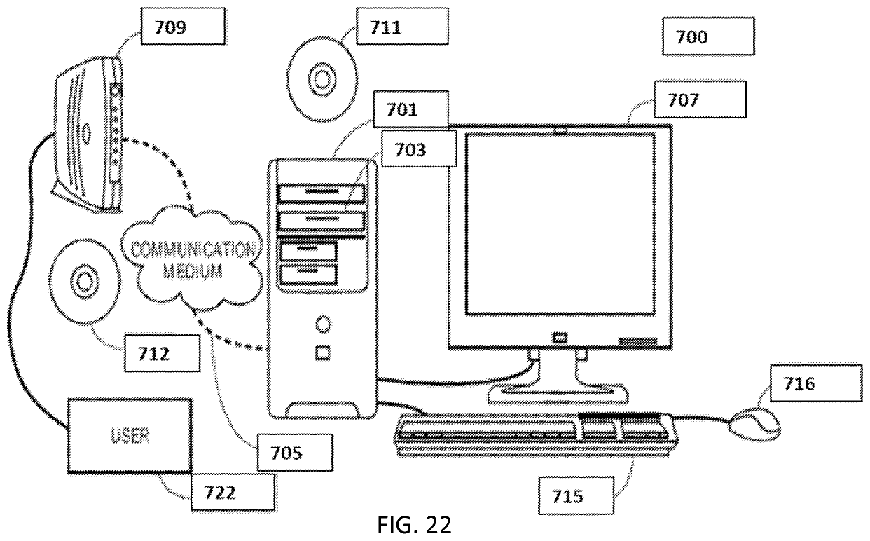

The computer system 700 illustrated in FIG. 22 can be understood as a logical apparatus that can read instructions from media 711 and/or a network port 705, which can optionally be connected to server 709 having fixed media 712. The system, such as shown in FIG. 22 can include a CPU 701, disk drives 703, optional input devices such as keyboard 715 and/or mouse 716 and optional monitor 707. Data communication can be achieved through the indicated communication medium to a server at a local or a remote location. The communication medium can include any means of transmitting and/or receiving data. For example, the communication medium can be a network connection, a wireless connection, an intranet connection, or an internet connection. Such a connection can provide for communication over the World Wide Web. It is envisioned that data relating to the present disclosure can be transmitted over such networks or connections for reception and/or review by a party 722 as illustrated in FIG. 22.

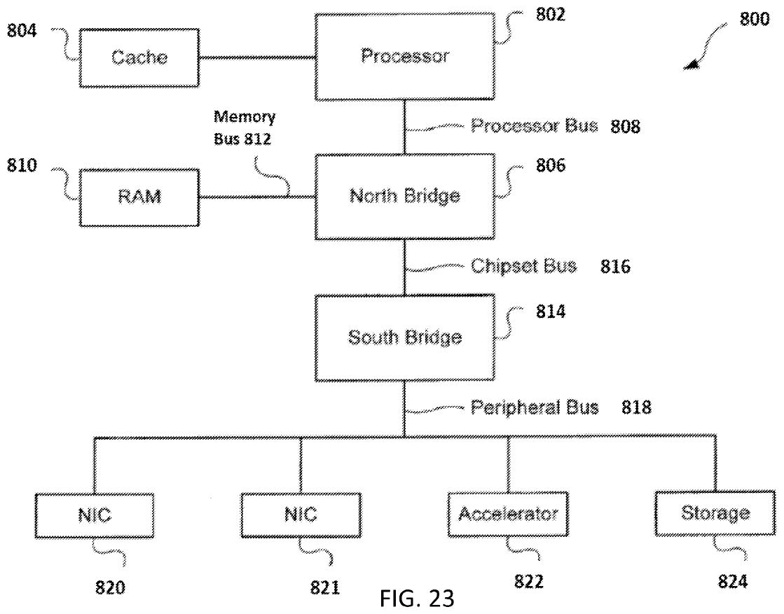

Provided herein is a block diagram illustrating a first example architecture of a computer system 800 that can be used in connection with example instances of the present disclosure as shown in FIG. 23. As depicted in FIG. 23, the example computer system can include a processor 802 for processing instructions. Non-limiting examples of processors include: Intel Xeon™ processor, AMD Opteron™ processor, Samsung 8-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 23, a high-speed cache 804 can be connected to, or incorporated in, the processor 802 to provide a high speed memory for instructions or data that have been recently, or are frequently, used by processor 802. The processor 802 is connected to a north bridge 806 by a processor bus 808. The north bridge 806 is connected to random access memory (RAM) 810 by a memory bus 812 and manages access to the RAM 810 by the processor 802. The north bridge 806 is also connected to a south bridge 88 by a chipset bus 816. The south bridge 814 is, in turn, connected to a peripheral bus 818. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 818. In some alternative architectures, the functionality of the north bridge can be incorporated into the processor instead of using a separate north bridge chip. In some instances, system 800 can include an accelerator card 822 attached to the peripheral bus 818. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 824 and can be loaded into RAM 810 and/or cache 804 for use by the processor. The system 800 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present invention. In this example, system 800 also includes network interface cards (NICs) 820 and 821 connected to the peripheral bus for providing network interfaces to external storage, such as Network Attached Storage (NAS) and other computer systems that can be used for distributed parallel processing.

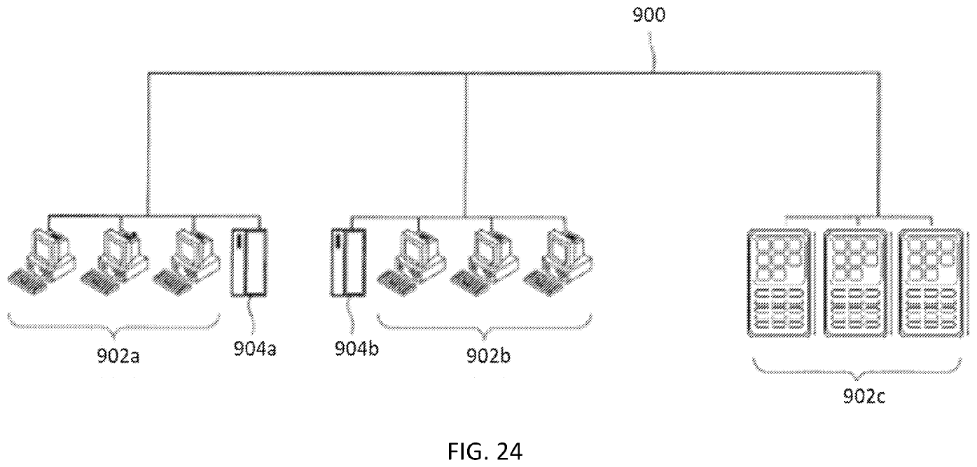

Provided herein is a diagram showing a network 900 with a plurality of computer systems 902a, and 902b, a plurality of cell phones and personal data assistants 902c, and Network Attached Storage (NAS) 904a, and 904b as shown in FIG. 24. In example instances, systems 902a, 902b, and 902c can manage data storage and optimize data access for data stored in Network Attached Storage (NAS) 904a and 904b. A mathematical model can be used for the data and be evaluated using distributed parallel processing across computer systems 902a, and 902b, and cell phone and personal data assistant systems 902c. Computer systems 902a, and 902b, and cell phone and personal data assistant systems 902c can also provide parallel processing for adaptive data restructuring of the data stored in Network Attached Storage (NAS) 904a and 904b. FIG. 24 illustrates an example only, and a wide variety of other computer architectures and systems can be used in conjunction with the various instances of the present invention. For example, a blade server can be used to provide parallel processing. Processor blades can be connected through a back plane to provide parallel processing. Storage can also be connected to the back plane or as Network Attached Storage (NAS) through a separate network interface.

In some example instances, processors can maintain separate memory spaces and transmit data through network interfaces, back plane, or other connectors for parallel processing by other processors. In other instances, some or all of the processors can use a shared virtual address memory space.

In some cases, a method or system disclosed herein can comprise transmission of data to or receipt of data from a database, such as an Electronic Medical Record (EMR) database. In some cases, data transferred to or from a database can comprise medical, biographical, and/or demographic information of one or more individuals (e.g., patients) of a population (e.g., a population included in the practice of a method or system described herein).

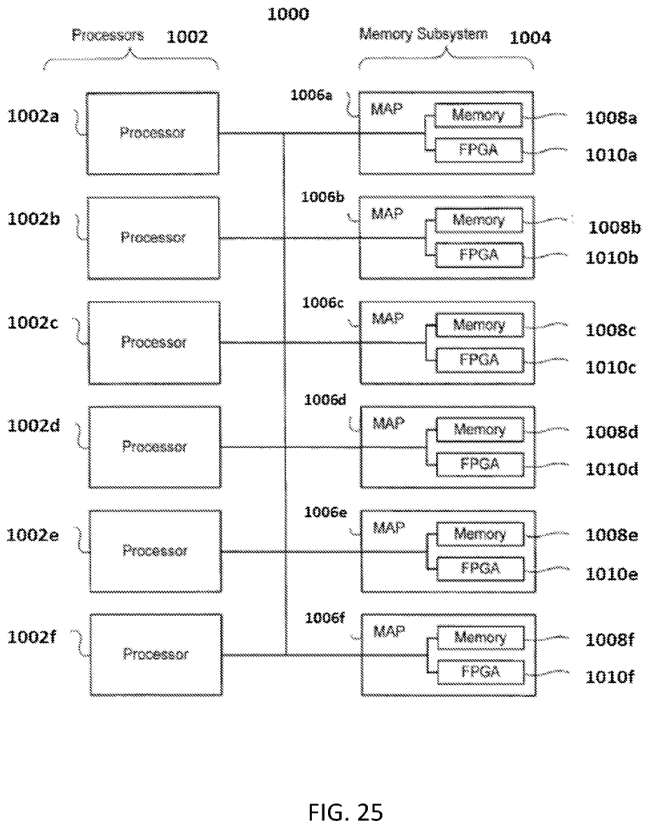

Provided herein is a block diagram of a multiprocessor computer system 1000 using a shared virtual address memory space as illustrated in FIG. 25 in accordance with an example embodiment. The system includes a plurality of processors 1002 comprising 1002a-f that can access a shared memory subsystem 1004. The system incorporates a plurality of programmable hardware memory algorithm processors (MAPs) 1006a-f in the memory subsystem 1004. Each MAP 1006a-f can comprise a memory 1008a-f and one or more field programmable gate arrays (FPGAs) 1010a-f. The MAP provides a configurable functional unit and particular algorithms or portions of algorithms can be provided to the FPGAs 1010a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1008a-f, allowing it to execute tasks independently of, and asynchronously from the respective microprocessor 1002a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 25, system on chips (SOCs), application specific integrated circuits (ASICs), or other processing and logic elements. For example, a system or device described herein can be implemented with hardware acceleration through the use of a hardware accelerator card, such as accelerator card 722 illustrated in FIG. 22.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

As used herein, the term “hitpicking” is defined as identifying hits based on assay data (e.g., Ct values) and consolidating (e.g., “picking” or physically moving from a first well of a well plate to a second well of the same well plate or of another well plate) samples (or nucleic acids isolated from samples) that have been identified as positive for the presence of a nucleic acid sequence of interest (e.g., “hits”) into a new labware in an identifiable or predetermined format. For instance, “hitpicking” can comprise identifying and selecting hits based on assay data (e.g., Ct values) from one plate and rearranging the hits on another plate. In some embodiments, the hits can be rearranged in order of increasing Ct values for analysis. In some embodiments, “hitpicking” can be automated. In some embodiments, “hitpicking” can utilize a machine or a robot.

As used herein, the terms “tagment” or “tagmentation” are defined as processing of nucleic acid molecules, often comprising addition (e.g., via ligation or enzymatic molecular amplification, or covalent bonding) of one or more sequencing adapters to the nucleic acid molecules and optionally comprising cleavage or breakage of the nucleic acid molecules), for example, in preparation for molecular sequencing analysis. Tagmentation can sometimes be performed simultaneously with or sequentially with “barcoding”. “Barcoding” can comprise the addition of a unique molecular identifier (UMI) to one or more nucleic acid molecules prior to sequencing (e.g., prior to pooling of nucleic acid molecule libraries). In many cases, barcoding can allow raw sequencing results to be correlated to specific libraries or samples.

As used herein, the term “STAT sample” is defined as a sample identified for urgent processing (e.g., comprising RNA isolation) and/or analysis (e.g., RT-qPCR analysis and/or molecular sequencing), for instance in a hospital setting.

As used herein, the term “production line sample” is defined as a sample that is not identified for urgent processing (e.g., comprising RNA isolation) and/or analysis (e.g., RT-qPCR analysis and/or molecular sequencing).

As used herein, the terms “patient,” “patients,” “subject,” or “subjects” mean animals, especially humans, that is/are the subject of one or more steps of a method or of the practice of a system component described in at least some embodiments, but not necessarily all embodiments, of the present disclosure. For example, a “patient,” “patients,” “subject,” or “subjects” can refer to one or more animals (e.g., humans) from which a sample is derived for use in one or more steps of a method described herein or in the practice of at least a portion of a one or more components of a system described herein.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### OTHER EMBODIMENTS

In some aspects, provided herein is a method for reconstructing a genome of a pathogen, comprising: determining a Ct value for a plurality of RNA molecules isolated from a sample; transporting the plurality of RNA molecules at a temperature no greater than −20° C. from a first location to a second location; performing a RT-PCR protocol on RNA from a biological sample to generate a plurality of cDNA molecules; sequencing the modified nucleic acid at the second location to determine a nucleotide sequence of all or a portion of the plurality of cDNA molecules.

In some aspects, provided herein is a system for a high-throughput nucleic acid sequencing and analysis, the system comprising: a first analysis module configured to determine a plurality of respective amplification cycle threshold (Ct) values for each of a first plurality of nucleic acid populations; a second analysis module configured to perform a one-step amplification protocol on a subset of the first plurality of nucleic acid populations; a third analysis module comprising a molecular sequencer configured to determine a first plurality of nucleic acid sequences corresponding to each population of the subset of the first plurality of nucleic acid populations; and a computer system configured to determine a rate of mutation in a pathogen based on the first plurality of nucleic acid sequences.

In some aspects, provided herein is a method for reconstructing a genome of a pathogen, comprising: determining an amplification cycle threshold (Ct) value for a plurality of ribonucleic acid (RNA) molecules isolated from a biological sample; transporting the plurality of RNA molecules at a temperature no greater than −20° C. from a first location to a second location; performing a reverse transcription polymerase chain reaction (RT-PCR) protocol on at least a portion of the plurality of RNA molecules to generate a plurality of complementary deoxyribonucleic acid (cDNA) molecules; and sequencing all or a portion of the plurality of cDNA molecules at the second location to determine nucleotide sequences of the all or the portion of the plurality of cDNA molecules.

In some aspects, provided herein is a method for reconstructing a genome of a pathogen, comprising: determining an amplification cycle threshold (Ct) value for a plurality of ribonucleic acid (RNA) molecules isolated from a biological sample; transporting the plurality of RNA molecules at a temperature no greater than −20° C. from a first location to a second location; performing a reverse transcription polymerase chain reaction (RT-PCR) protocol on at least a portion of the plurality of RNA molecules to generate a plurality of complementary deoxyribonucleic acid (cDNA) molecules; sequencing at least a portion of the plurality of cDNA molecules at the second location to determine nucleotide sequences of the at least the portion of the plurality of cDNA molecules; and analyzing the nucleotide sequences to reconstruct the genome of the pathogen.

In some embodiments, the plurality of cDNA molecules comprises a modified nucleic acid. In some embodiments, the sequencing comprises sequencing the modified nucleic acid at the second location to determine the nucleotide sequences.

In some embodiments, performing the RT-PCR protocol comprises a one-step RT-PCR protocol. In some embodiments, an input volume of RNA for the one-step RT-PCR protocol is at least about 5 microliters. In some embodiments, an elongation temperature of the one-step RT-PCR protocol is about 60.5° C. In some embodiments, an elongation time of the one-step RT-PCR protocol is about 3 minutes. In some embodiments, performing the one-step RT-PCR protocol comprises use of an RMv1 or RMv2 primer set. In some embodiments, performing the one-step RT-PCR protocol comprises use of a primer, the primer comprising a 5′ end modification. In some embodiments, the primer is biotinylated at the 5′ end.

In some embodiments, the method further comprises tagmenting at least a portion of the plurality of cDNA molecules. In some embodiments, each cDNA molecule of the plurality of cDNA molecules is tagmented with polyethylene glycol (PEG).

In some embodiments, the method further comprises performing hybrid capture on at least a portion of the tagmented cDNA molecules. In some embodiments, the method further comprises performing hybrid capture on a portion of the tagmented cDNA molecules derived from RNA molecules with a Ct value greater than about 27, as determined using a real-time quantitative PCR (RT-qPCR) assay.

In some embodiments, performing the RT-PCR protocol comprises use of an exonuclease with a processivity of at least about 60 nucleotides per second. In some embodiments, performing the RT-PCR protocol comprises use of Taq polymerase. In some embodiments, performing the RT-PCR protocol comprises use of a tiling primer that is longer than an A400 primer. In some embodiments, performing the RT-PCR protocol comprises use of an A1200 primer.

In some embodiments, the biological sample comprises a saliva sample, a blood sample, a urine sample, a cell lysate, or a tissue biopsy sample. In some embodiments, the biological sample is obtained or derived from a subject or a patient.

In some embodiments, the method further comprises storing data generated by the method in a database accessible through a communication medium. In some embodiments, the data comprises the Ct values for the plurality of RNA molecules isolated from the biological sample or the nucleotide sequences. In some embodiments, the communication medium comprises a network connection, a wireless connection, an intranet connection, or an internet connection.

In some aspects, provided herein, is a system for high-throughput nucleic acid sequencing and analysis, the system comprising: a first analysis module configured to determine a plurality of respective amplification cycle threshold (Ct) values for each of a first plurality of nucleic acid populations; a second analysis module configured to perform a one-step amplification protocol on a subset of the first plurality of nucleic acid populations; a third analysis module comprising a molecular sequencer configured to determine a first plurality of nucleic acid sequences corresponding to each population of the subset of the first plurality of nucleic acid populations; and a computer system configured to determine a rate of mutation in a pathogen based at least in part on the first plurality of nucleic acid sequences.

In some embodiments, the subset of the first plurality of nucleic acid populations is selected based at least in part on the respective Ct values determined by the first analysis module.

In some embodiments, the system further comprises a processing device configured to prepare the subset of the first plurality of nucleic acid populations for analysis by the second analysis module. In some embodiments, preparing the subset of the first plurality of nucleic acid populations for analysis comprises automatically transferring the subset of the first plurality of nucleic acid populations to respective wells of a processing container.

In some embodiments, the processing device is further configured to prepare a subset of a second plurality of nucleic acid populations for analysis by the second analysis module based at least in part on respective Ct values of the second plurality of nucleic acid populations determined by a fourth analysis module, wherein each population of the subset of the second plurality of nucleic acid populations has a Ct value less than a threshold Ct value.

In some embodiments, the third analysis module is further configured to determine a second plurality of nucleic acid sequences corresponding to each population of the subset of the second plurality of nucleic acid populations.

In some embodiments, each of the first plurality of nucleic acid populations is derived from a respective subject of a first population of subjects, and wherein each of the second plurality of nucleic acid populations is derived from a respective subject of a second population of subjects, at least one of the first population of subjects and at least one of the second population of subjects having an unknown risk of exposure to a pathogen of interest.

In some embodiments, the subject of the first population of subjects is located in a different geographical area compared to the subject of the second population of subjects.

In some embodiments, the computer system is further configured to determine a rate of transmission of the pathogen based at least in part on the first plurality of nucleic acid sequences and the second plurality of nucleic acid sequences. In some embodiments, the rate of mutation is based at least in part on the first plurality of nucleic acid sequences and the second plurality of nucleic acid sequences.

In some embodiments, the Ct value is about 37. In some embodiments, an input volume of the first plurality of nucleic acid populations for the one-step amplification protocol is at least about 5 microliters. In some embodiments, an elongation temperature of the one-step amplification protocol is about 60.5° C. In some embodiments, an elongation time of the one-step amplification protocol is about 3 minutes. In some embodiments, performing the one-step amplification protocol comprises use of an RMv1 or RMv2 primer set. In some embodiments, performing the one-step amplification protocol comprises use of a primer, the primer comprising a 5′ end modification. In some embodiments, the primer is biotinylated at the 5′ end.

In some embodiments, performing the one-step amplification protocol on the subset of the first plurality of nucleic acid populations generates a plurality of nucleic acid molecules.

In some embodiments, the system further comprises tagmenting at least a portion of the plurality of nucleic acid molecules. In some embodiments, each nucleic acid molecule of the plurality of nucleic acid molecules is tagmented with polyethylene glycol (PEG). In some embodiments, the system further comprises performing hybrid capture on at least a portion of tagmented nucleic acid molecules.

In some embodiments, performing the one-step amplification protocol comprises use of an exonuclease with a processivity of at least about 60 nucleotides per second. In some embodiments, performing the one-step amplification protocol comprises use of Taq polymerase. In some embodiments, performing the one-step amplification protocol comprises use of a tiling primer that is longer than an A400 primer. In some embodiments, performing the one-step amplification protocol comprises use of an A1200 primer. In some embodiments, the one-step amplification protocol comprises a one-step RT-PCR protocol.

In some embodiments, the system further comprises a database accessible through a communication medium. In some embodiments, the database is configured to store data generated by the first analysis module, the second analysis module, the third analysis module, the fourth analysis module, or the computer system. In some embodiments, the data comprises Ct values for each of the first plurality of nucleic acid populations, the first plurality of nucleic acid sequences corresponding to each population of the subset of the first plurality of nucleic acid populations, or the rate of mutation in the pathogen based at least in part on the first plurality of nucleic acid sequences. In some embodiments, the communication medium comprises a network connection, a wireless connection, an intranet connection, or an internet connection.

In some aspect, provided herein is a system for high-throughput nucleic acid sequencing and analysis, the system comprising: a first analysis module configured to determine a plurality of respective amplification cycle threshold (Ct) values for each of a first plurality of nucleic acid populations; a second analysis module configured to select a subset of the first plurality of nucleic acid populations; a third analysis module configured to perform a one-step amplification protocol on the subset of the first plurality of nucleic acid populations; a fourth analysis module comprising a molecular sequencer configured to determine a first plurality of nucleic acid sequences corresponding to each population of the subset of the first plurality of nucleic acid populations; and a computer system configured to determine a mutation in a pathogen based at least in part on the first plurality of nucleic acid sequences.

In some embodiments, the second analysis module is further configured to rearrange the subset of the first plurality of nucleic acid populations. In some embodiments, the computer system is further configured to determine a rate of mutation in a pathogen based at least in part on the first plurality of nucleic acid sequences.

In some embodiments, the subset of the first plurality of nucleic acid populations is selected based at least in part on the respective Ct values determined by the first analysis module. In some embodiments, the system further comprises a database accessible through a communication medium. In some embodiments, the respective Ct values are stored in the database. In some embodiments, the respective Ct values stored in the database are accessible by scanning a barcode on a source container comprising the first plurality of nucleic acid populations. In some embodiments, the subset of the first plurality of nucleic acid populations is rearranged in order of increasing Ct values for analysis by the third analysis module.

In some embodiments, the system further comprises a fifth analysis module configured to determine a plurality of respective amplification cycle threshold (Ct) values for each of a second plurality of nucleic acid populations.

In some embodiments, a subset of the second plurality of nucleic acid populations is selected based at least in part on the respective Ct values determined by the fifth analysis module. In some embodiments, the subset of the second plurality of nucleic acid populations is rearranged by the second analysis module in order of increasing Ct values for analysis by the third analysis module.

In some embodiments, the second analysis module comprises a processing device. In some embodiments, the processing device comprises a liquid handling system. In some embodiments, the processing device comprises an automated system. In some embodiments, the automated system comprises a computer processor programmed to automatically transfer the subset of the first plurality of nucleic acid populations or the subset of the second plurality of nucleic acid populations to respective wells of a processing container in order of increasing Ct values.

In some embodiments, the third analysis module is further configured to determine a second plurality of nucleic acid sequences corresponding to each population of the subset of the second plurality of nucleic acid populations. In some embodiments, each of the first plurality of nucleic acid populations is derived from a respective subject of a first population of subjects, and wherein each of the second plurality of nucleic acid populations is derived from a respective subject of a second population of subjects.

In some embodiments, at least one of the first population of subjects and at least one of the second population of subjects have an unknown risk of exposure to a pathogen of interest. In some embodiments, the subject of the first population of subjects is located in a different geographical area compared to the subject of the second population of subjects.

In some embodiments, the computer system is further configured to determine a rate of transmission of the pathogen based at least in part on the first plurality of nucleic acid sequences and the second plurality of nucleic acid sequences. In some embodiments, the rate of mutation is based at least in part on the first plurality of nucleic acid sequences and the second plurality of nucleic acid sequences. In some embodiments, the Ct value is no more than about 37.

In some embodiments, an input volume of the first plurality of nucleic acid populations for the one-step amplification protocol is at least about 5 microliters. In some embodiments, an elongation temperature of the one-step amplification protocol is about 60.5° C. In some embodiments, an elongation time of the one-step amplification protocol is about 3 minutes. In some embodiments, performing the one-step amplification protocol comprises use of an RMv1 or RMv2 primer set. In some embodiments, performing the one-step amplification protocol comprises use of a primer comprising a 5′ end modification. In some embodiments, the primer is biotinylated at the 5′ end.

In some embodiments, the one-step amplification protocol comprises a one-step RT-PCR protocol. In some embodiments, performing the one-step RT-PCR protocol on the subset of the first plurality of nucleic acid populations or the subset of the second plurality of nucleic acid populations generates cDNA molecules. In some embodiments, performing the one-step RT-PCR protocol on the subset of the first plurality of nucleic acid populations and the subset of the second plurality of nucleic acid populations generates cDNA molecules.

In some embodiments, the system further comprises a sixth analysis module for tagmenting each cDNA molecule of the subset of the first plurality of nucleic acid populations and/or each cDNA molecule of the subset of the second plurality of nucleic acid populations. In some embodiments, each cDNA molecule of the subset of the first plurality of nucleic acid populations and/or each cDNA molecule of the subset of the second plurality of nucleic acid populations is tagmented with polyethylene glycol (PEG).

In some embodiments, the systems further comprises a seventh analysis module configured to perform hybrid capture on at least a portion of tagmented cDNA molecules. In some embodiments, performing the one-step amplification protocol comprises use of an exonuclease with a processivity of at least about 60 nucleotides per second. In some embodiments, performing the one-step amplification protocol comprises use of Taq polymerase. In some embodiments, performing the one-step amplification protocol comprises use of a tiling primer that is longer than an A400 primer. In some embodiments, performing the one-step amplification protocol comprises use of an A1200 primer.

In some embodiments, the database is configured to store data generated by the first analysis module, the second analysis module, the third analysis module, the fourth analysis module, the fifth analysis module, the sixth analysis module, the seventh analysis module, or the computer system. In some embodiments, the data comprises the plurality of respective amplification Ct values for each of the first plurality of nucleic acid populations, the first plurality of nucleic acid sequences corresponding to each population of the subset of the first plurality of nucleic acid populations, or the mutation in the pathogen based at least in part on the first plurality of nucleic acid sequences. In some embodiments, the communication medium comprises a network connection, a wireless connection, an intranet connection, or an internet connection.

### EXAMPLES

These examples are provided for illustrative purposes only and not to limit the scope of the claims provided herein.

### Example 1: RNA Transport, Storage, and Hitpicking

This example shows methods and systems for nucleic acid transport, storage, and selection in methods and systems for nucleic acid analysis.

Preservation of RNA sample quality before library preparation for sequencing can be the crucial step of systems and methods for analysis of nucleic acids described herein. Following quantitative PCR (qPCR) testing at a first location (e.g., a location 100 remote from a second location 200 at which sequencing analysis may be performed, e.g., as shown in FIG. 1A and FIG. 1C), RNA plates containing SARS-CoV-2 positive samples with Ct value of 34 and below were identified and barcoded for hitpicking. Using a Tecan Fluent Automation Workstation, only the positive samples were hitpicked using a custom hitpicking scripts or computer processor programs that were generated to reformat all positive samples into a single 384-well PP Echo plate starting with the lowest Ct sample in A1 and ascending row by row. In addition, a positive control was added at wells B2 and P22 of the 384-well plate, and a negative control in well P24, such that quadrant 4 of a given plate could be tested for quality control and contain both positive controls and the negative control, along with 95 test samples arranged in order of increasing Ct value (as determined by qPCR at the first location). After collection of enough positive samples for a sequencing run, the plates were transported on dry ice to an analysis facility at the second location. Temperature trackers were used to ensure maintenance of temperatures below −20° C. Upon arrival, the plates were thawed on ice until they were ready for RT-PCR on the same day.

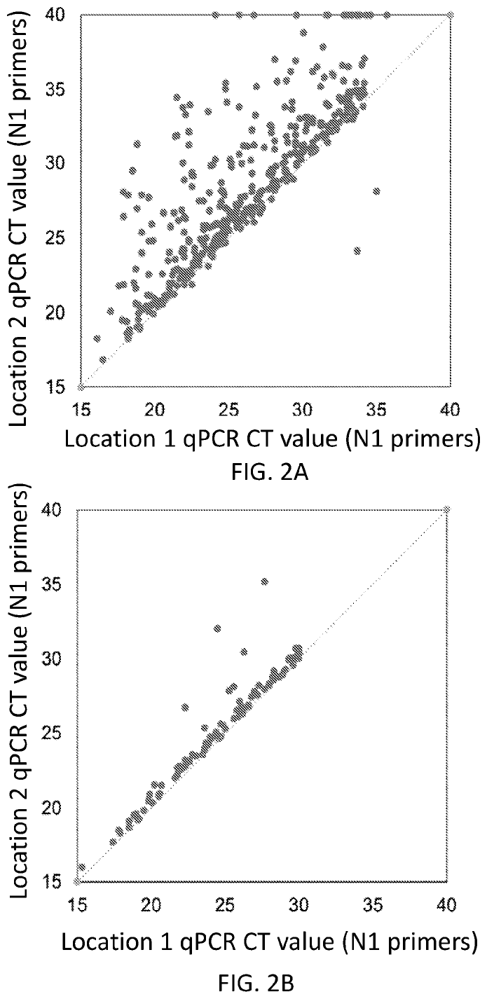

It was observed that RNA degradation caused by even short times at ambient temperature or freeze-thaw cycles, as may occur during transport, storage, or hitpicking, dramatically reduces the reconstruction success rate for SARS-CoV-2 positive samples and can be detected as a shift in Ct (e.g., observed cycle threshold) value for these samples (FIG. 2A). This is true especially for samples with low RNA concentration (e.g., having high Ct values, as determined by qPCR). To avoid these issues, a strictly regimented process for temperature control during shipping, storage, and hitpicking to preserve RNA quality between the diagnostic lab and sequencing lab facilities. To confirm that these methods preserve RNA quality, RT-qPCR using both the CDC N1 and N2 primer-probe sets were regularly performed after hitpicking at the analysis facility at the second location and compared to those reported at the remote analysis facility at the first location. Ct values for both primer-probe sets generally aligned closely with the original values obtained at the remote testing facility, indicating that the implemented protocols for transport, storage, and hitpicking of samples were successful in preserving RNA quality for downstream sequencing (FIG. 2B). Ct values are routinely measured for samples arriving at the sequencing facility after being diagnosed and hitpicked at the clinical lab. As shown in FIG. 2A, samples which have been degraded due to improper handling such as extended time at ambient temperature or multiple freeze-thaws can be identified by deviation from the Ct value obtained at the clinical lab, seen as scatterplot points which rise above the identity line. As shown in FIG. 2B, samples which are handled properly should deviate little if at all from the original Ct values. In some cases, deviation from expected Ct values observed in samples after receipt at the second location can be addressed by evaluating adherence to established protocols and, if necessary, corrective measures can be implemented.

### Example 2: RNA Extraction of STAT Samples

This example shows embodiments of methods and systems for obtaining nucleic acid molecules from a sample obtained from a subject.

Reagent plates including binding plate, wash plate, 80% ethanol plate, and elution plate were prepared in advance for RNA extraction using ThermoScientific KingFisher™ Flex instrument which has capability to extract 96 samples in parallel. Binding plate was prepared by mixing 33 mL of pre-made Teknova Binding Solution (4M Guanidine Thiocyanate Buffer, 10 mM Tris-HCl, 1 mM EDTA, 20% PEG 8000, 5% Tween-20, storage at 25° C.) with well-vortexed SpeedBead Magnetic Particles in a 50 mL Falcon tube. The tube was then inverted five times to ensure even distribution of beads and then set on the rocker for 5 minutes. 300 μL of the final solution was dispensed into each well of a KingFisher™ 96 deep well plate and stored at 25° C. until use. The wash plate and ethanol plates were prepared by adding 500 μL of Teknova Wash Solution and 80% ethanol solution, respectively, into each well of KingFisher™ 96 deep well plates and then stored at 25° C. An elution plate was prepared by dispensing 50 μL of TE Buffer (pH 8.4) into each well of a KingFisher™ 96 Well (200 uL) plate and stored at 25° C. Once all the reagents were ready, a biosafety cabinet work surface was thoroughly disinfected with Eliminase and 70% ethanol.

Biological specimens (e.g., STAT samples) were collected locally (e.g., at or local to the second location 200, for example, as shown in FIG. 1B), were treated with 200 microliters (μL) of Proteinase K and before being transferred to individual vials for transport to. Proteinase K-treated patient samples were carefully transferred into each well of the binding plate and pipetted thoroughly with the binding solution. The sample plate and the reagent plates were placed on the KingFisher™ Flex instrument in accordance with the instructions of in-house protocol created for the RNA extraction, and a fresh set of tip combs was loaded before starting the protocol. After the 44 minute automated RNA extraction assay run was completed by the KingFisher™ Flex liquid-handling instrument, the extracted RNA sample plate was sealed immediately and stored in a 2° C.-8° C. refrigerator until RT-PCR reaction on the same day.

### Example 3: Sequencing Production Line Samples

Patient samples were tested for COVID-19 the same day as collection at a first location 100 using a qPCR-based method (see, U.S. patent application Ser. No. 17/478,415, which is incorporated herein by reference for all purposes). The extracted RNA plates used for testing were stored at −20° C. freezer immediately after testing until they are delivered on dry ice to a second location for sequencing. The first step of the sequencing pipeline upon receiving extracted RNA samples at a second location from a first location is reformatting only the SARS-CoV-2 positive samples into a new 384-well destination plate with an automated process called hitpicking using a liquid handling system (e.g., an Opentrons OT-2 system or Tecan system). The positive RNA samples are then transferred into two plates containing RT-PCR master mix to allow separate reactions with Primer Pool 1 (P1) and Primer Pool 2 (P2). P1 and P2 plates were subjected to RT-PCR overnight and then pooled together into a single plate diluting the samples to approximately 0.5 ng/uL. The samples were then tagmented using Illumina Tagment Mix and barcoded. The barcoded samples are pooled together into a single tube, column purified, diluted, and denatured before being loaded onto the Illumina sequencer.

### Example 4: RT-PCR Enzyme Selection

This example shows data comparing effects of selection of a one-step or a two-step RT-PCR protocol on genome sequencing quality.

A Midnight primer set (comprising RMv1 or RMv2 primer sets, as described herein) and protocol (see, Freed et al., Biology Methods and Protocols, Volume 5, Issue 1, 2020, bpaa014 (which is hereby incorporated by reference in its entirety for all purposes)) was chosen as a starting point to reduce the proportion of the genome to which primers anneal, mitigating the risk of fragment dropouts due to accumulation of mutations in those regions. In some cases, a 400 bp amplicon or 1200 bp amplicon primer set (e.g., ARTIC network 400 bp version 3 primer pool (IDT catalog number: 10006788) or 1200 bp ARTIC Network primer set) described in Freed et al., Biology Methods and Protocols, Volume 5, Issue 1, 2020, bpaa014 (which is hereby incorporated by reference in its entirety for all purposes)) can be used. This protocol calls for using a two-step RT-PCR.

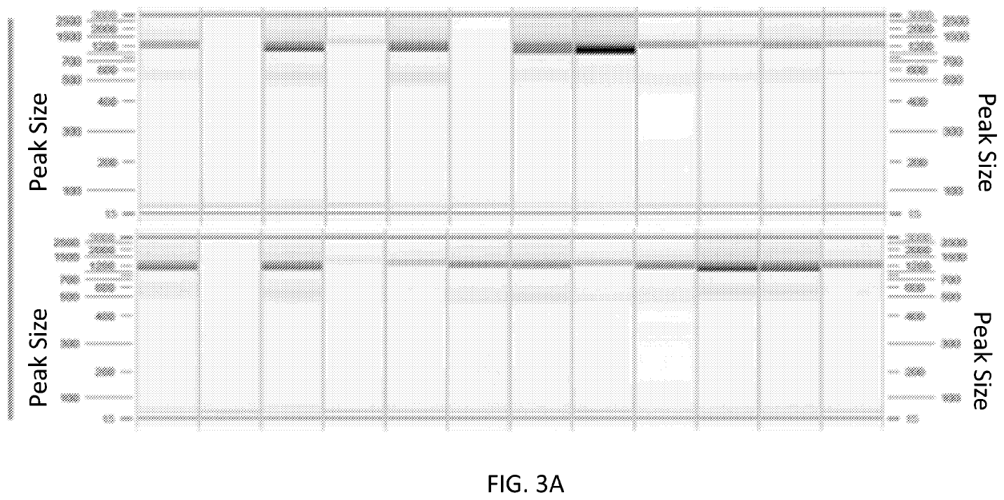

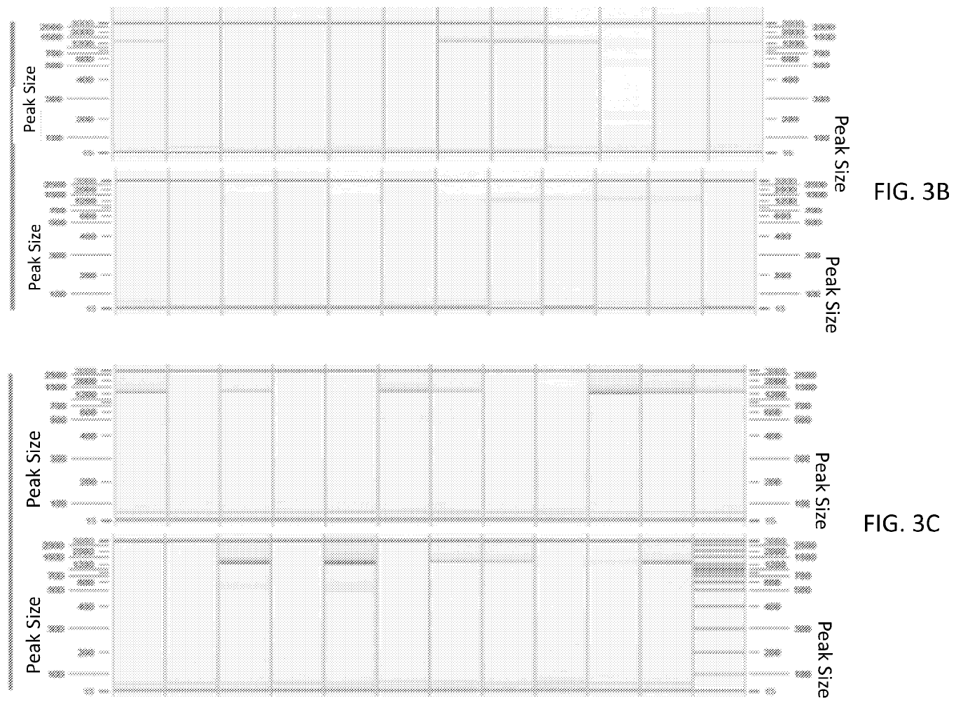

The effect of one-step RT-PCR kits to reduce labor, cost, and system runtime, and possibly improve performance was also investigated. Thus, a side-by-side comparison of a one-step RT-PCR approach to a two-step RT-PCR approach was designed. In order to determine a specific one-step PCR kit for use in side-by-side comparison experiments with the LunaScript+Q5 two-step protocol, a variety of 1-step kits were compared. One-step RT-PCR kits were compared in a genomic PCR test of SARS-CoV-2 positive samples. Takara One Step PrimeScript III (#RR600A) (FIG. 3A), Invitrogen SuperScript™ IV One-Step (#12594025) (FIG. 3B), and NEB OneTaq One-Step (#E5315S) (FIG. 3C) kits were tested for their ability to produce genomic cDNA in an A1200 RT-PCR of 24 SARS-CoV-2 positive samples. Resulting cDNA was run on a Qiaxcel high sensitivity capillary gel electrophoresis cartridge. Takara outperformed all other kits, producing more robust bands across the broadest range of samples compared to other kits.

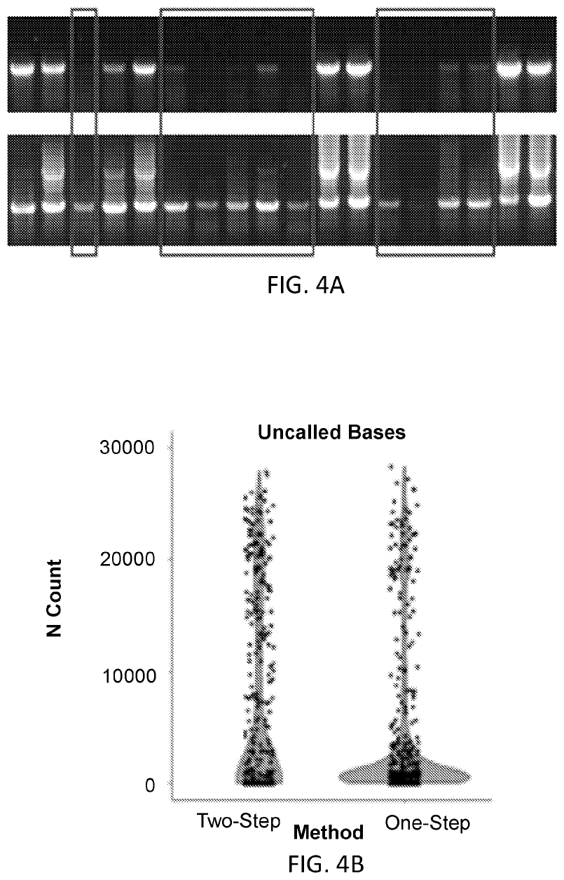

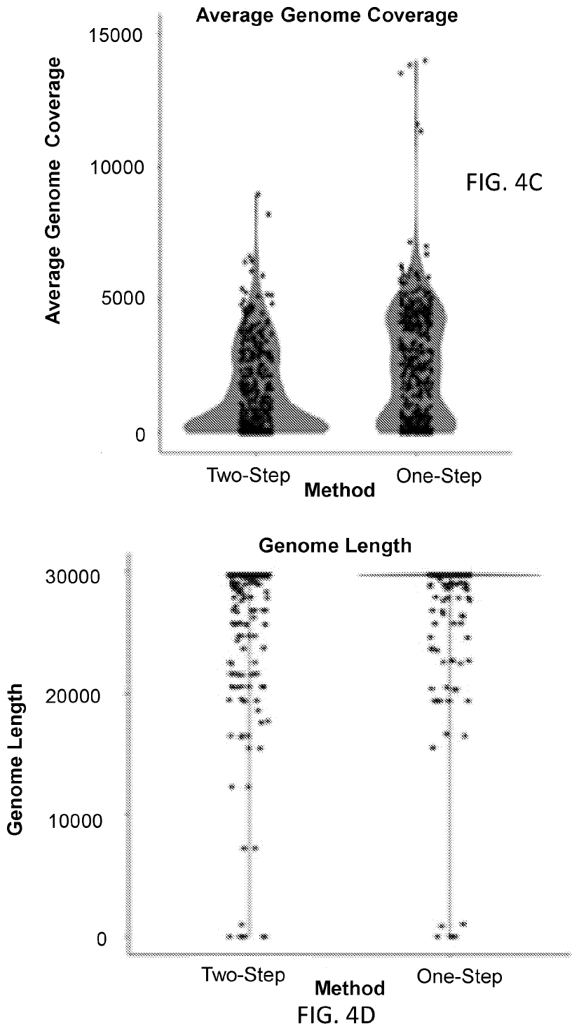

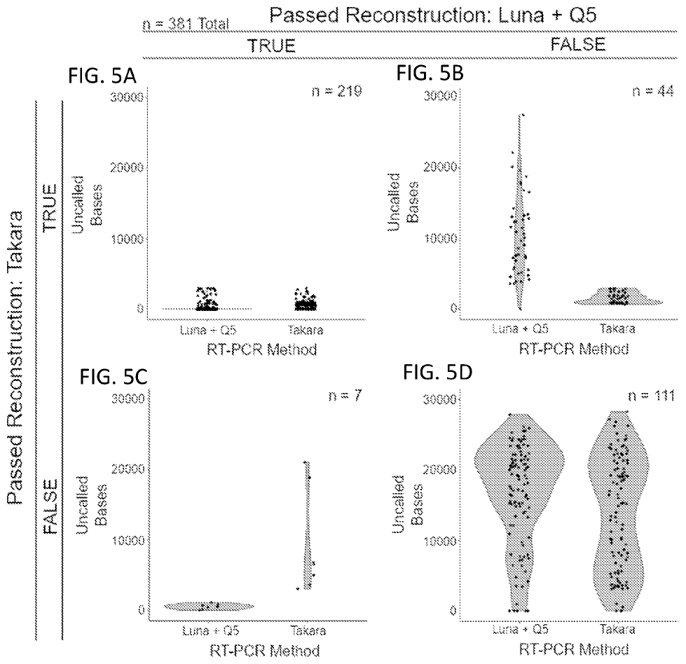

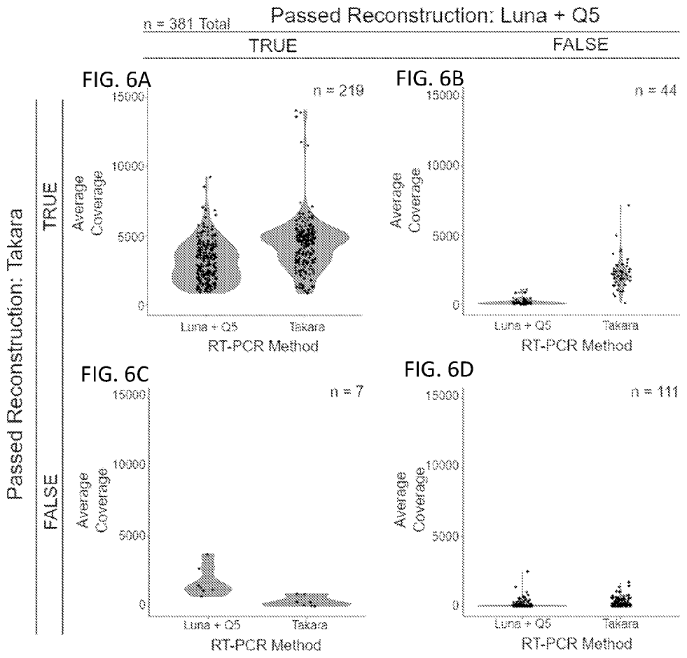

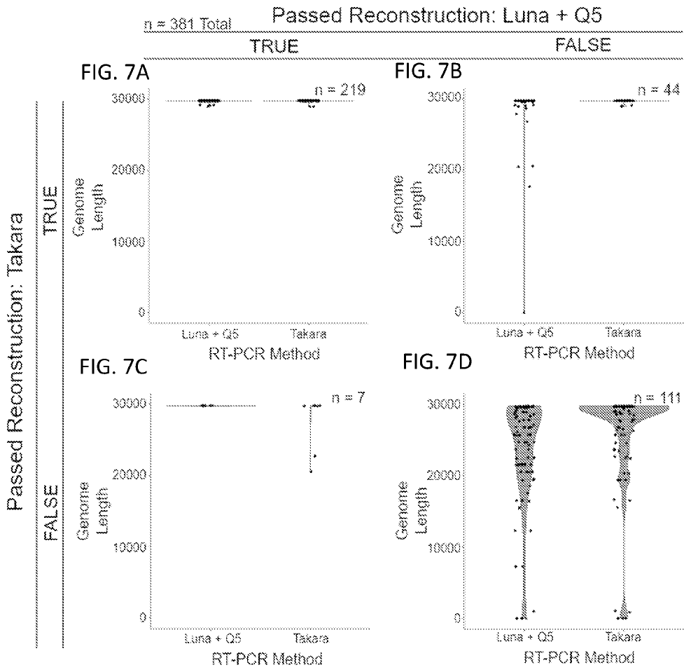

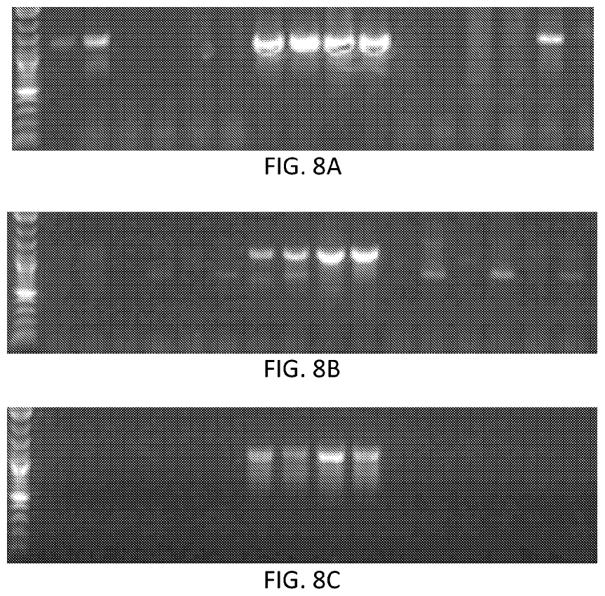

The one-step method (Takara) chosen to move forward with a side-by-side comparison against the two-step protocol was found to be superior in generating the desired viral amplicons where it yielded better results in the multiplexed PCR reactions of viral SARS-CoV-2 samples, yielding stronger bands in many samples where both succeeded, and also yielding bands in many samples where the two-step method did not. Analysis of RT-PCR kit and parameters for the generation of SARS-CoV-2 cDNA for genome sequencing included RT-PCR analysis of 18 SARS-CoV-2 RNA samples was performed using the two-step Lunascript+Q5 protocol described in the original protocol using the Midnight primer set (FIG. 4A, top) or the Takara 1-step kit (FIG. 4A bottom). For 9 of 18 samples, the 1-step kit performed better at generating a visible band than the 2-step protocol. 384 SARS-CoV-2 positive RNA samples were sequenced using both the 2-step and Takara 1-step protocols. In addition to generating more reconstructed genomes, the Takara 1-step protocol generated fewer uncalled bases (FIG. 4B), more average genome coverage (FIG. 4C) In the test of 384 RNA SARS-CoV-2 positive RNA samples, the number of genomes reconstructed by the Takara one-step kit was higher than the two-step protocol, allowing reconstruction of 263 genomes compared to 226 genomes out of 384 RNA samples attempted across a wide range of Ct values. The one-step kit also performed better across a variety of other metrics, including yielding fewer uncalled bases (FIG. 4B, FIGS. 5A-5D), greater average genome coverage (FIG. 4C, FIGS. 6A-6D), and more complete genomes (FIG. 4D, FIGS. 7A-7D) and longer consensus genomes than the 2-step protocol. This was true even and especially in cases where the genome construction did not meet our stringent criteria for a passable genome, but still yielded usable genome information (FIG. 5D, FIG. 6D, and FIG. 7D).

In some cases, the one-step method is higher than the two-step protocol, allowing reconstruction of 263 genomes compared to 226 genomes for the two-step method out of 384 RNA samples attempted across a wide range of Ct values. The one-step method also performed better across a variety of other metrics, including yielding fewer missing nucleotide fragments (FIG. 4B), greater average genome coverage (FIG. 4C), and more complete genomes (FIG. 4D), even and especially in cases where the genome construction did not meet the criteria for a passable genome, but still yielded usable genome information. However, the one-step method had a slightly higher error-rate compared to the two-step protocol. Since these mutations occur randomly through the genome, and the same mutation is not likely to occur across many genomes, they do little to compromise the overall quality of the genomic data generated. In some cases, it can be advantageous to utilize a one-step amplification protocol with methods or systems described herein, for example, due to the savings in cost, labor, and time, the greater number of genomes constructed, and the substantially higher average quality of the genomes produced with the one-step protocol that can be realized with such modifications.

### Example 5: Comparison of Different One-Step RT-PCR Master Mixes and Two-Step RT Polymerases

This example shows comparison of one-step and two-step RT-PCR systems and methods for nucleic acid analysis.

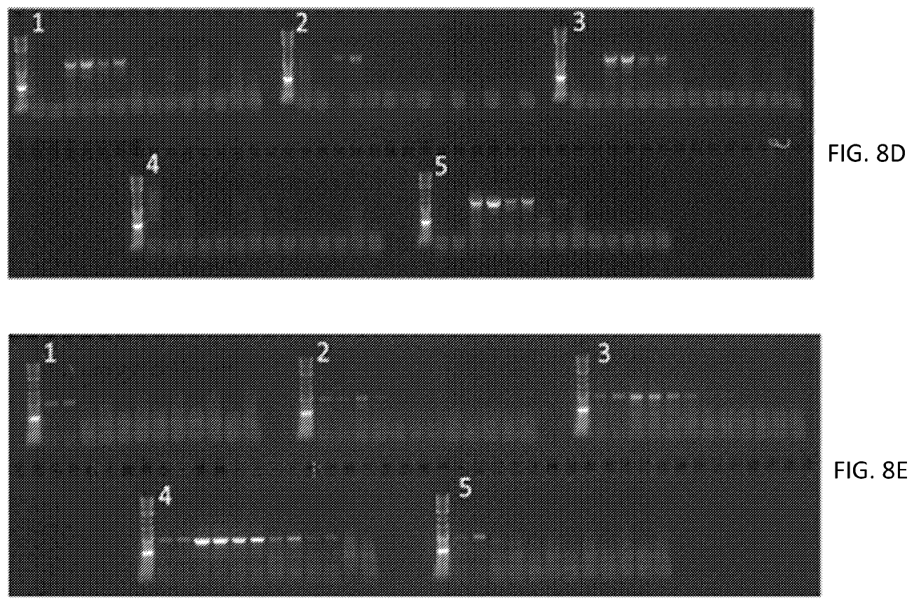

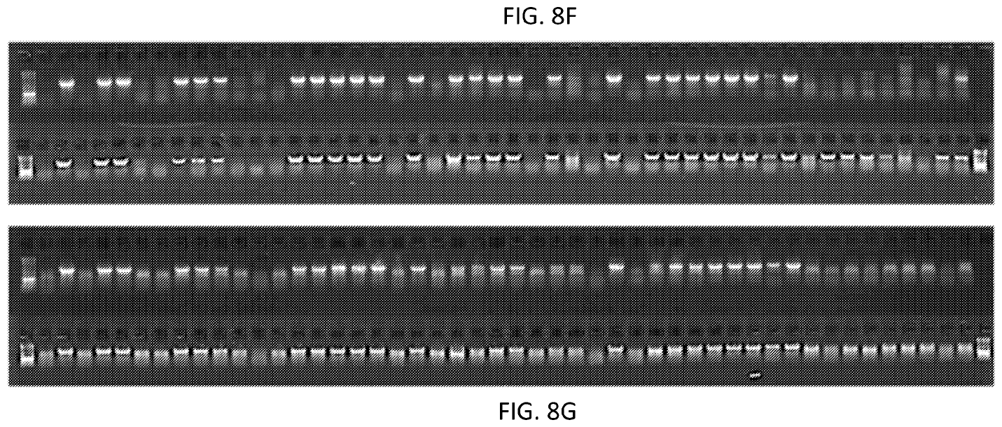

Some example embodiments have tested various one-step RT-PCR master mixes and reverse transcriptase for two-step RT PCR. The one-step RT-PCR master mixes, according to some example embodiments, have tests including a Takara one-step RT-PCR master mix (FIG. 8A), Thermo Fisher Superscript SSIV one-step RT-PCR master mix (FIG. 8B), Agilent AffinityScript one-step RT-PCR master mix (data not shown), and Qiagen OneStep Ahead one-step RT-PCR master mix (FIG. 8C). Takara one-step RT-PCR out-performed all other one-step RT-PCR master mixes in terms of sensitivity and yields.

With two-step RT-PCR, some example embodiments compare various reverse transcriptases using either random primer or gene specific primer as RT primers. FIG. 8D shows two-step RT-PCR results using various reverse transcriptase (RT) enzymes and random sequence primers for RT-PCR assays (panel 1 represents results from RT-PCR performed with Thermo Fisher Superscript IV Vilo RT enzyme; panel 2 represents results from RT-PCR performed with Roche Transcriptor RT enzyme; panel 3 represents results from RT-PCR performed with New England Biolabs (NEB) MMLV RT enzyme; panel 4 represents results from RT-PCR performed with NEB WarmStart reverse transcriptase; and panel 5 represents results from RT-PCR performed with Thermo Fisher Maxima H− reverse transcriptase). FIG. 8E shows two-step RT-PCR results using various reverse transcriptase (RT) enzymes and target gene-specific sequence primers for RT-PCR assays (panel 1 represents results from RT-PCR performed with Thermo Fisher Superscript IV (SSIV) Vilo RT enzyme; panel 2 represents results from RT-PCR performed with Roche Transcriptor RT enzyme; panel 3 represents results from RT-PCR performed with New England Biolabs (NEB) MMLV RT enzyme; panel 4 represents results from RT-PCR performed with NEB WarmStart reverse transcriptase; and panel 5 represents results from RT-PCR performed with Thermo Fisher Maxima H− reverse transcriptase). Individual reverse transcriptase (RT) enzymes were observed to perform differently depending on whether random primers or gene specific primers are used. Thermo Scientific's Maxima H⁻ reverse transcriptase was observed to perform the best among the enzymes tested when random primers were used (see, panel 5 of FIG. 8D) and NEB WarmStart reverse transcriptase was observed to perform the best when gene specific primers were used (see, panel 4 of FIG. 8E).

Some example embodiments also compared RT master mixes—Lunascript and SSIV Vilo master mixes. The Thermo Fisher SSIV Vilo RT master mix (FIG. 8F) performed better than the New England Biolabs (NEB) Lunascript RT master mix (FIG. 8G, lower panel).

### Example 6: Analysis of the Effects of PCR Elongation Time and Elongation Temperature on Sequencing Results

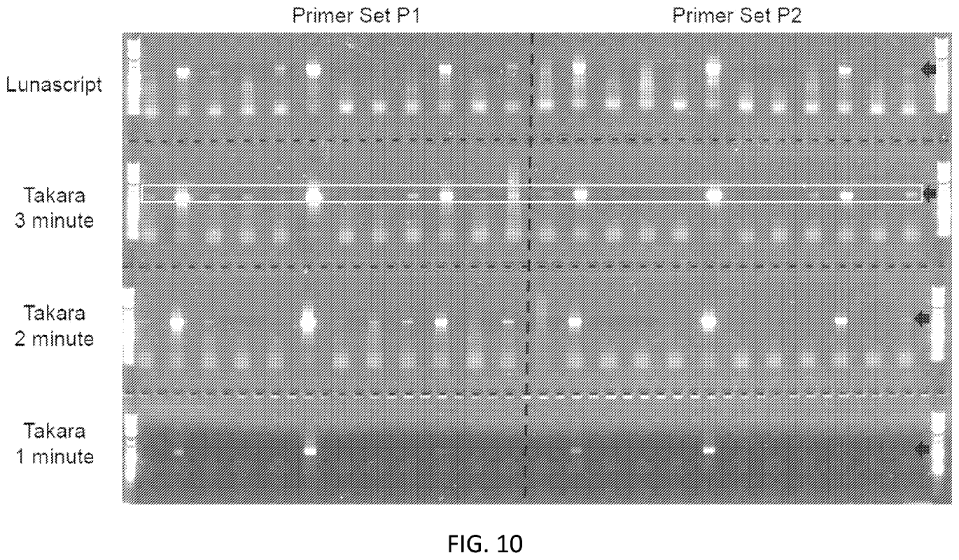

To further improve pipeline performance while reducing protocol runtime, we sought to optimize the PCR cycling parameters. First, a temperature gradient was performed during the elongation cycle of the RT-PCR reaction utilizing the Takara PrimeScript kit to determine the optimal extension temperature. The extension temperature of 61° C. was found to be optimal for producing an RT-PCR band from the most samples while minimizing off-target product formation (FIG. 9). Next, extension times were investigated for an ability to improve nucleic acid efficiency without compromising the ability to produce products from marginal, high Ct value samples. It was found that extension times of 3 minutes produced the highest quality results (FIG. 10). These findings were incorporated into cycling protocols (e.g., two-step cycling protocols) for RT-PCR of the SARS-CoV-2 genome.

### Example 7: Analysis of the Effects of PCR Input Volume on Sequencing Results

The effect of input volume of sample RNA added to the RT-PCR reaction was investigated. While increasing total RNA in the reaction should intuitively improve RT-PCR performance of low-concentration samples, residual contaminants from the RNA extraction procedure can also inhibit RT-PCR. Therefore, it was necessary to determine the RNA volume that would maximize the RNA input without compromising RT-PCR performance. When tested on STAT samples that were directly received and extracted at the LIC site using Kingfisher™ Flex (Thermo Scientific), there was a clear difference in the number of uncalled bases, average coverage, and genome length for different input volumes, and optimal results were observed with higher input volume of 5 μL (FIG. 11A, FIG. 11B, FIG. 11C). RT-PCR was performed on 37 samples using either 1 μL or 5 μL of sample as input in the reaction. The 5 μL reactions were found to have fewer uncalled bases (FIG. 11A), greater average coverage (FIG. 11B), and longer average genome length (FIG. 11A) than achieved in the 1 μL cases.

The differing RNA input results between the STAT and the production line samples can likely be attributed to a combination of extraction mechanisms, difference in elution volumes, and differences in sample handling and timing before RT-PCR. Consequently, this parameter should be experimentally investigated for any new genome sequencing operation which may use alternative extraction methods or instruments.

### Example 8: Primer Pool Rebalancing

Multiplexed PCR reactions may suffer from uneven amplicon representation, for example, due to factors that can include variable strength of annealing of primers to template, formation of primer dimers for specific primers, and/or inherent instability of genomic regions. To inform rebalancing of primer concentrations in the multiplexed PCR reaction, the representation of each amplicon in successfully reconstructed genomes using equimolar ratios of the Midnight primer set up to that point were input into a previously described equation to output a modified weight for each primer pair in pools P1 and P2. This ratio was used to determine the volume of each 100 μM primer to input into the first iteration of the remixed primer sets (RMv1). These remixed primer pools were used alongside the equimolar primer pools to sequence 67 STAT samples using 1 or 3 μL of RNA template as input. In some cases (e.g., to avoid a complete primer redesign), primer concentrations can be rebalanced in the multiplexed reaction, which can make amplicon representation in sequenced pools more even. In some cases, samples can be sequenced using equimolar primer ratios and the average representation of each amplicon in the sequenced pool can be assessed. Values can be input into an equation to output a modified weight for each primer set in the reaction, which can be used to calculate the volume of each primer to input into the first iteration of the remixed primer set (RMv1). In some embodiments, the primer can be present at (e.g., used at) a concentration of 100 μM.

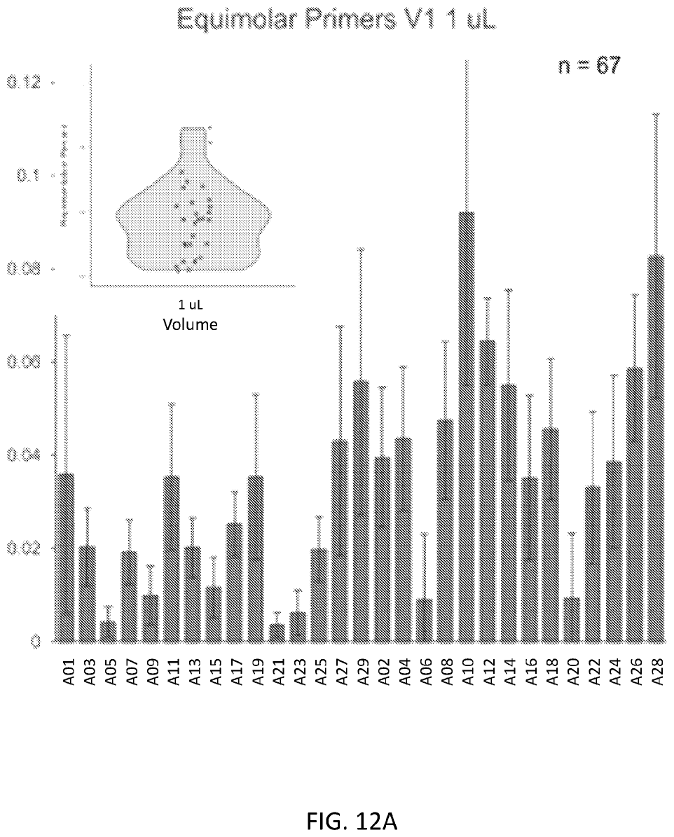

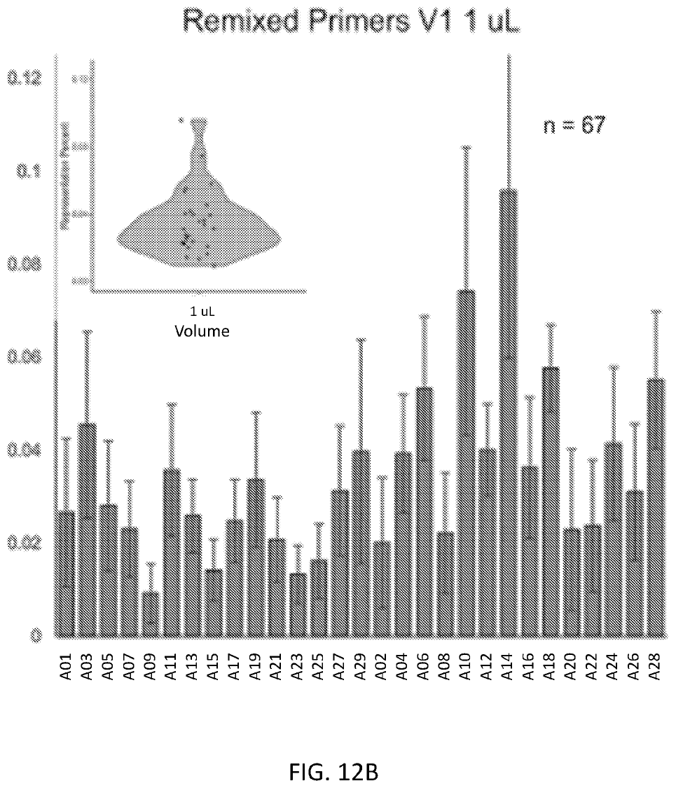

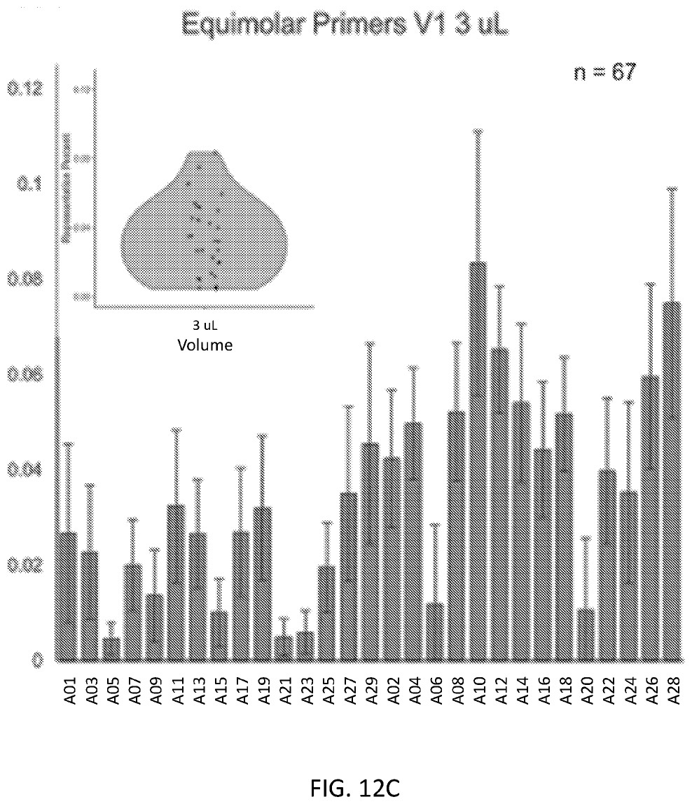

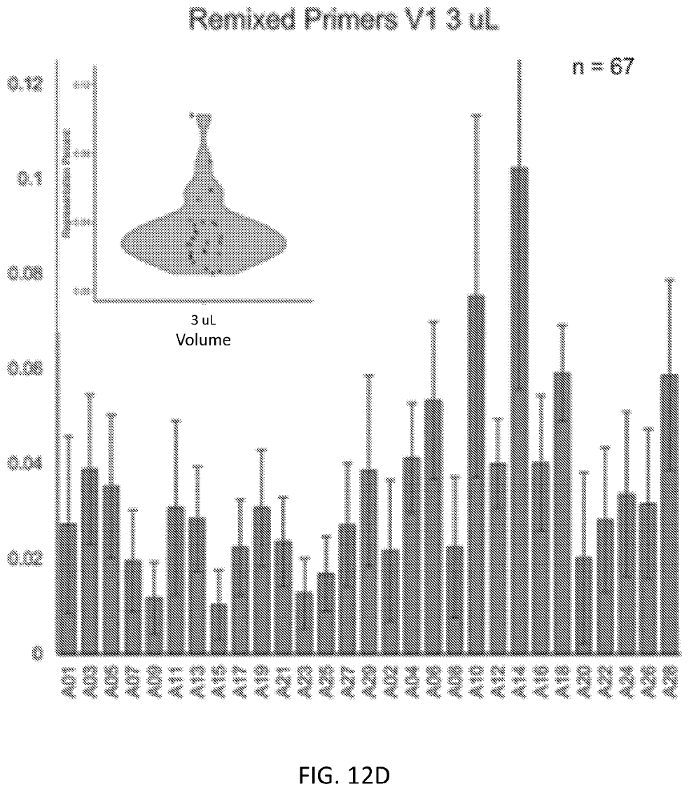

Primer concentrations for each primer pair were rebalanced to achieve more even representation of each genome fragment in the final sequenced libraries. While the spread of average amplicon representation remains similar between equimolar (FIG. 12A and FIG. 12B) and remixed (FIG. 12C and FIG. 12D) primer sets, most amplicons shift toward the desired average representation of each amplicon given a pool of 29 amplicons (˜3.45%). For both sets of samples, the evenness of representation for most amplicons was improved, and most importantly, amplicons A05, A21, A23, A06, and A20 were substantially improved in representation (FIG. 12A, FIG. 12B, FIG. 12C, FIG. 12D). In some cases, primer pool RMv1 can produce more evenness in amplicon representation for STAT samples, e.g., as compared to on production line samples, suggesting that RNA template integrity can be a significant factor in promoting the evenness of amplicon representation in the pool. A second iteration of primer rebalancing using the same strategy but the new average amplicon representations using RMv1 is performed, resulting in RMv2. In some cases, RMv1 can be used moving forward for both production and STAT samples, e.g., to reduce reagent costs and/or costs associated with labor. Table 2 shows values for RMv1 and RMv2 remixed primer sets. In the case a new variant of concern is identified, primer sets can be rebalanced in a similar fashion.

TABLE 2
RMv1 and RMv2 values

| Amplicon | RMv1 | RMv2 |
|---|---|---|
| P1_A01 | 1.042591 | 1.2450354 |
| P1_A03 | 1.037028 | 0.7106669 |
| P1_A05 | 1.867374 | 0.7769786 |
| P1_A07 | 1.076907 | 0.8999492 |
| P1_A09 | 1.255891 | 0.9442806 |
| P1_A11 | 0.919866 | 0.8157614 |
| P1_A13 | 1.081557 | 0.8659067 |
| P1_A15 | 1.156913 | 0.9020504 |
| P1_A17 | 1 | 0.8870008 |
| P1_A19 | 0.974278 | 0.8409597 |
| P1_A21 | 2.022967 | 0.9328681 |
| P1_A23 | 1.421984 | 0.9232586 |
| P1_A25 | 1.092013 | 1 |
| P1_A27 | 0.942341 | 0.9331577 |
| P1_A29 | 0.984575 | 0.9015649 |
| P2_A02 | 1.258761 | 0.9620844 |
| P2_A04 | 1.183071 | 0.899562 |
| P2_A06 | 3.36667 | 0.7189887 |
| P2_A08 | 1.010018 | 1 |
| P2_A10 | 0.884002 | 0.791678 |
| P2_A12 | 0.983079 | 0.8703884 |
| P2_A14 | 0.995058 | 0.8079585 |
| P2_A16 | 1.30835 | 0.7992964 |
| P2_A18 | 1.116233 | 0.7481119 |
| P2_A20 | 2.882298 | 0.8537968 |
| P2_A22 | 1.381209 | 0.8454907 |
| P2_A24 | 1.148861 | 0.814314 |
| P2_A26 | 0.971132 | 0.7331893 |
| P2_A28 | 0.964542 | 0.8000724 |
| | P1 sum | V2_PA_sum |
| | 71.50514 | 64.547656 |

### Example 9: Primer Modification

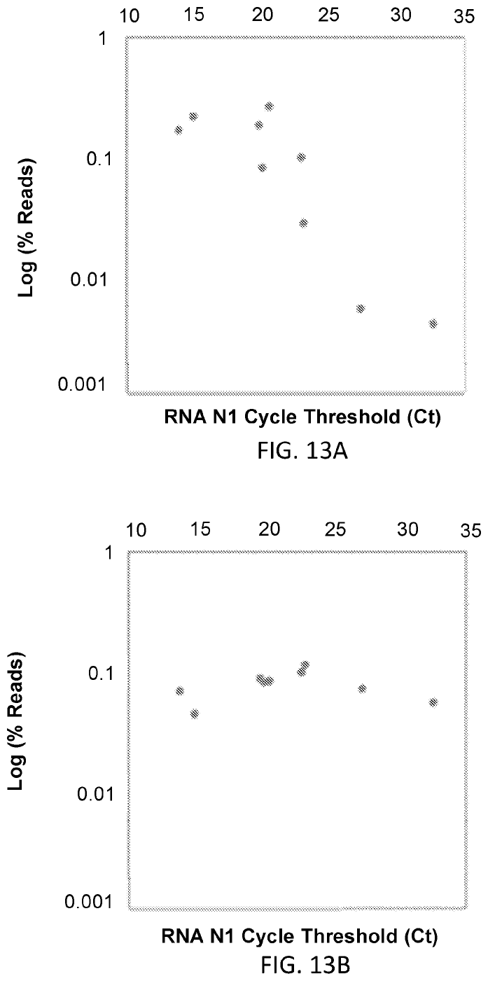

This example shows embodiments of PCR primers useful in methods and systems for nucleic acid analysis. Addition of molecules (e.g., polynucleotides, amino acids, carbohydrates, etc.) to the 5′ end of a primer can block (e.g., at least partially block) 5′ to 3′ exonuclease activity of a polymerase used in PCR assays in which the modified primer is used. In some cases, such effects on exonuclease activity can improve amplicon evenness. Effects of 5′ biotinylation of A1200 primers was investigated. Results shown in FIG. 13A, FIG. 13B, and FIG. 13C indicate that modification of the 5′ end of a primer can achieve improved sequencing results in methods and systems described herein. For example, FIG. 13B shows that percent of reads observed during sequencing were maintained for high Ct value samples in assays employing 5′-end biotinylated primers, whereas primers that were not biotinylated at the 5′ end (FIG. 13A) showed dropoff in reads at high Ct values. FIG. 13C shows a comparison of amplicon representation for 5′-biotinylated and 5′ non-biotinylated versions of P1 and P2 primers.

### Example 10: Incorporation of Touchdown PCR

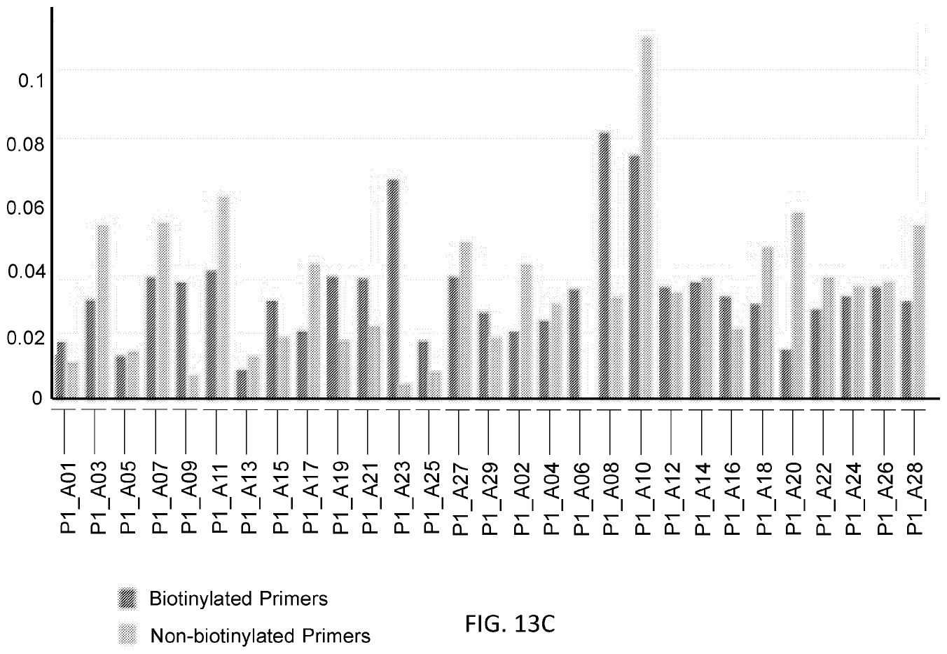

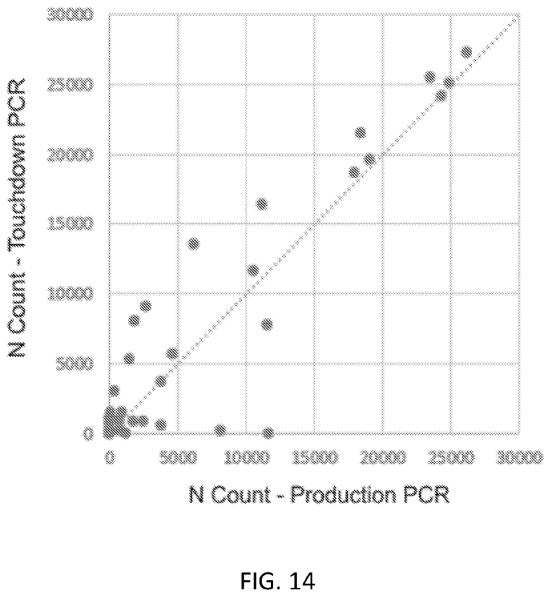

Touchdown PCR is a variant of PCR in which the annealing temperature in the first cycle of PCR is set far above the optimal annealing temperature for the primer, and it is incrementally lowered in each cycle until reaching the optimal temperature for the primer or enzyme5. This method can encourage the formation of the desired products by making unintended primer-template binding or primer-dimer interactions less favorable in the early cycles of PCR. We explored the effect of touchdown PCR to further improve the efficiency of our A1200 RT-PCR and overall genome reconstruction. We tested an initial elongation temperature of 72° C. and incrementally decreased the annealing temperature in each cycle to the previously determined optimal extension temperature (61° C.) in our 2-step PCR protocol Using a set of extracted RNA samples with Cts between 15 and 35, we carried out our standard RT-PCR and touchdown PCR in parallel, then proceeded with downstream library prep and sequencing. While some high-Ct samples appeared to benefit from touchdown PCR, we observed more undetermined bases in general across these samples compared to standard PCR when sequenced (FIG. 14). Based on these results, touchdown PCR was not implemented in our sequencing pipeline.

### Example 11: A1200 Amplicon RT-PCR of Hitpicked Positive RNA Plates

Mastermix plates were preassembled containing 5 μL of Takara One-Step Prime Script III 2× Mastermix in Thermo Scientific Armadillo clear 384-well PCR plates and stored at −20° C. For each plate of positive hitpicked samples, 1 or 3 μL of SARS-CoV-2 RNA and H2O to 10 μL was pipetted into two Mastermix plates using the Analytick Jena CyBio Felix robot. These plates were then transferred to the Echo 525 acoustic pipettor, where 50 nL of 100 μM P1 or P2 A1200 primer mix were dispensed to each well. Plates were then briefly spun down and cycled in Eppendorf Mastercycler X50t thermal cyclers using the following protocol: Reverse transcription reaction at 52° C. for 30 min followed by 35 cycles of 15 seconds denaturation at 95° C., 3 minutes annealing at 61° C. and a final cooling to 12° C.

### Example 12: Tagmentation of A1200 Amplicons

Positive RNA specimens between cycle threshold of 15-30 were selected from tested samples and cDNA for each specimen was generated using LunaScript RT SuperMix (NEB, MA) according to manufacturer protocol. To target SARS-CoV-2 specifically, cDNA for each specimen was amplified in two separate pools, 28- and 30-plex respectively, to generate 1200 bp of overlapping amplicons using Q5 2× Hot-Start Master Mix (NEB, MA). The resulting pools are combined in equal volume and enriched for full length 1200 bp product using a SPRI-based magnetic bead cleanup. Enriched amplicons are tagmented (Illumina, CA) and barcoded (IDT, IA) and paired-end sequenced on an Illumina MiSeq or NextSeq 550.

Tagmentation and barcoding add Illumina sequencing adapters and index sequences to the RT-PCR product so that multiple samples can be pooled and sequenced together. The input DNA and tagmentation enzyme (Nextera) ratio determines the library size. To achieve the optimal size distribution, different amounts of amplicon input and different tagmentation enzyme concentration were tested, and it was determined that tens of copies of the A1200 amplicon can be tagmented.

Illumina Tagment Mix was prepared by creating a master mix containing 0.25 μL of Tagment Enzyme with 1.25 μL Tagment buffer for each well. The mix was dispensed into Thermo Scientific Armadillo clear 384-well PCR plates using Formulatrix Mantis and placed on ice. Following RT-PCR, 2 μL of A1200 P1 and 2 μL of P2 reactions were pooled together and diluted 30-fold into 120 μL of nuclease-free water. This step was performed to bring the maximum predicted total concentration of the A1200 reaction products to below 0.5 ng/μL to enable efficient tagmentation. 1 μL of the diluted mixture was transferred to the tagmentation plate containing 1.5 μL Tagment Mix, and then placed in the thermocycler for tagmentation reaction. Tagmentation protocol is as follows: Thermocycler pre-heated to 55° C. before insertion of the plate, then tagmentation at 55° C. for 10 minutes.

### Example 13: Testing Different Amplicon Sizes for Tagmentation

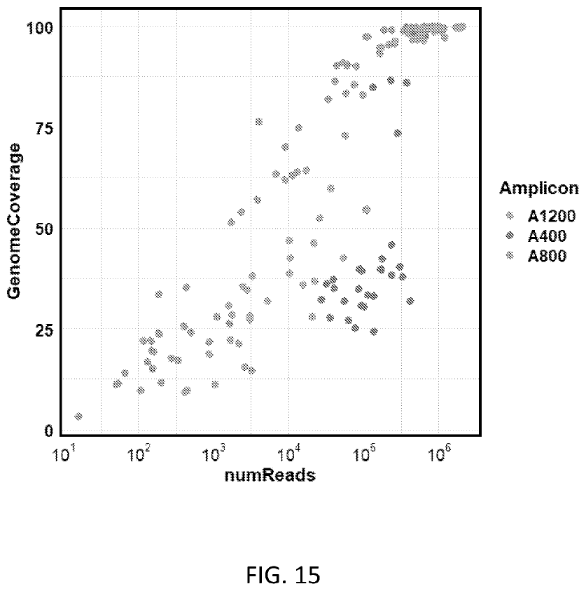

Some example embodiments designed and tested different amplicon sizes for tagmentation-based library preparation, including 400, 800 and 1200 bp amplicon sizes. All tested amplicon sizes are tagmented and generate good coverage for the COVID (e.g., SARS-CoV-2) genomes. The read depth required to cover 99% of the genome is similar among the different amplicon sizes. Although A800 seems to require slightly less depth than A1200 and A1200 than A400, the observed differences were not statistically significant. (FIG. 15).

### Example 14: Addition of Polyethylene Glycol (PEG) to Improve Tagmentation Efficiency and Recovery of High Ct Samples

PEG has been used in tagmentation reactions for NGS library construction under diluted reaction conditions in large reaction volumes (20 μl). To test the effect of PEG on tagmentation of A1200 amplicons in our miniaturized reaction, tagmentation was carried out with or without 8% PEG 8000. When the standard quantity of transposome was used in the reactions, 8% PEG 8000 had no effect on the tagmentation reaction as the tagmented and amplified PCR product with or without PEG showed identical size distribution (Data not shown). However, when the amount of transposome used is reduced by 10 fold, we observed a large effect with longer incubation time (20 min vs. 10 min. with normal amount of transposome). The tagmented and amplified PCR product showed normal PCR product in the presence of PEG and no PCR product in the absence of PEG. Such effect of PEG 8000 was not observed with higher or lower amounts of PEG (16% or 4%, data not shown).

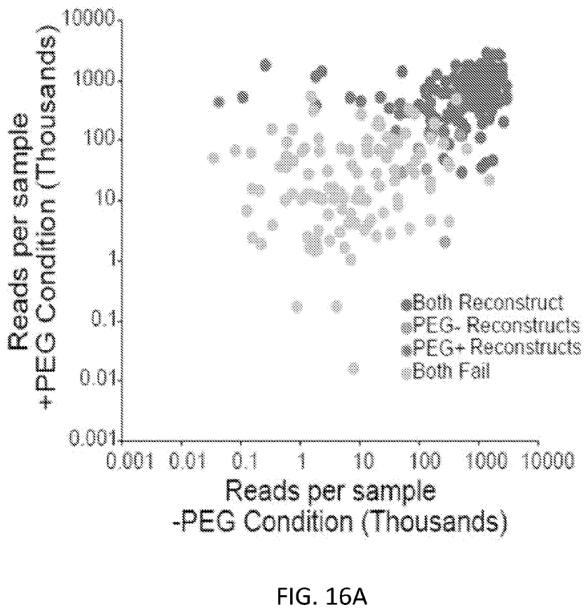

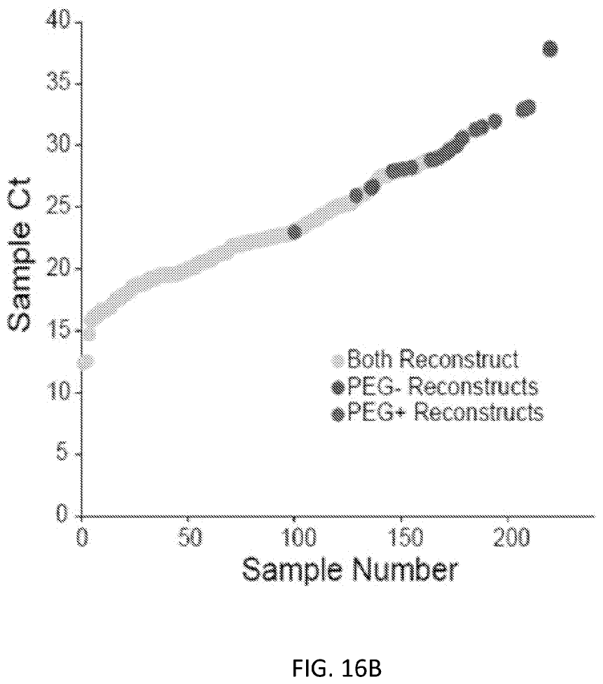

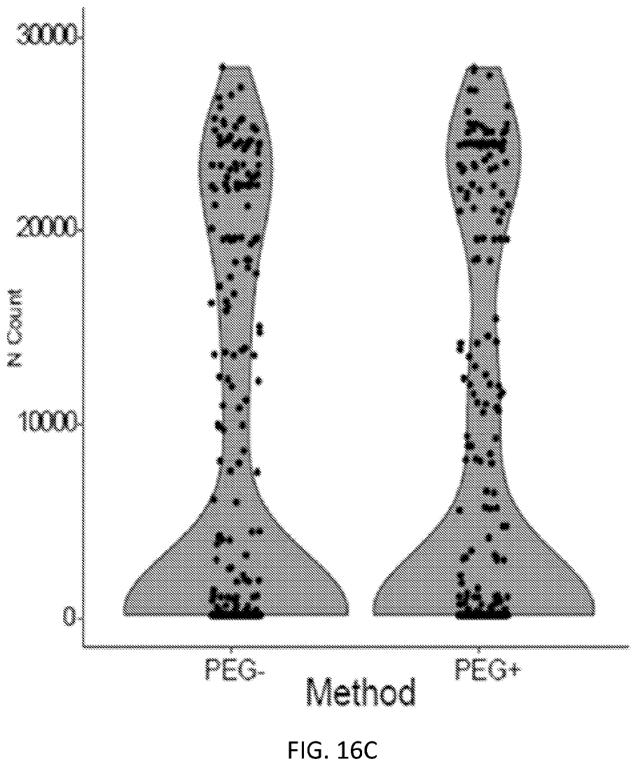

RT-PCR was performed on a set of 288 SARS-CoV-2 positive samples, and were tagmented in the presence and absence of 8% PEG 8000 and with 10-fold reduced tagmentation enzyme (FIG. 16A). Samples tagmented with PEG in general achieved greater reads per sample, and 16 genomes reconstructed only in the PEG+ condition (green), compared to only 6 in the PEG− case (orange). Reconstructed samples from this experiment were plotted by Ct and colored by whether they reconstructed in both treatments (light blue), only in the PEG− condition (red), or only in the PEG+ condition (blue) (FIG. 16B). Samples which reconstruct only in the PEG+ case are highly biased toward high-Ct samples, suggesting that these samples may preferentially reconstruct due to molecular crowding induced by PEG. Samples from the PEG− and PEG+ conditions are displayed in violin plots of the number of uncalled bases with individual points represented (FIG. 16C). There is no statistically significant difference in the overall number of uncalled bases per sample.

Tagmentation in the presence of PEG also somewhat normalized sample reads between low Ct and high Ct samples (see figure, the number of reads were boosted for samples with lower read numbers when tagmented without PEG). As a result, we observed a similar or slightly better full genome reconstruction rate for higher Ct samples in comparison to tagmentation without PEG despite the 10-fold decrease in quantity of transposome used (FIG. 16A, FIG. 16B). These genome reconstructions also did not have a significantly different number of uncalled bases despite the 10-fold decrease in quantity of transposome used (two-sided t-test, p=0.9692) (FIG. 16C). This technique provides a way to reduce the cost of tagmentation significantly without sacrificing library quality. In our pipeline, this technique has been primarily used to preserve tagmentation enzyme in instances where supply chain issues have made procuring new enzyme in a timely manner challenging, or when increases in sample volume (during the Omicron surge) have rapidly depleted our stocks.

Tagmentation, or transposase-mediated fragmentation and adapter-insertion, is the most efficient method available to transform dsDNA into fragmented libraries ready for barcoding. Tagmentation kits are one of the most expensive enzymatic steps of sequencing pipelines. Some example embodiments have miniaturized the tagmentation reaction to reduce per sample sequencing cost. With the addition of a PEG additive into the miniaturized tagmentation reaction, some example embodiments demonstrate a further significant reduction in the amount of enzyme required (a reduction of 90%), while maintaining the performance of the sequencing results in terms of successful genome reconstruction (FIG. 16A) and genome completeness (FIG. 16C).

### Example 15: Barcoding of Tagmented DNA and Library Amplification

Barcoding MasterMix was prepared by mixing 5 μL of 2× Kapa Hifi HotStart ReadyMix with 2.25 μL of nuclease-free water for each well. Following tagmentation, Kapa Mix was dispensed directly onto the tagmentation plate using MANTIS with high-volume chips. Using Echo 525 acoustic dispensor, 400 nL of 100 μM n7xx and 400 nL of 100 μM of n5xx oligo primers (Illumina) were dispensed consecutively onto the sample plate. Following addition of the primers, the barcoded samples—now 10 μL total volume—were placed on the thermocycler for barcoding PCR reaction. The optimized barcoding PCR reactions are as follows: 72° C. for 5 minutes, 98° C. for 5 minutes, 13 cycles of 10 sec of denaturation at 98° C., 30 sec of annealing at 66° C., and 30 seconds of extension at 72° C. Final extension was done for 5 min at 72° C. before cooling to 10° C. For each of the 384-well plates, the final libraries are categorized as either low Ct (Ct<27) or high Ct (Ct>27) according to their measured Ct values. 2 μL from each library is pooled into its respective low-bind tubes. All samples are purified using DNA Clean & Concentrator-5 (Zymo Research) and eluted in approximately 50 μL of elution buffer.

### Example 16: Library Pooling and Loading onto Illumina Sequencer

When pooling many barcoded DNA libraries into a single sequencing run, normalization of the concentration of each library is an important step to ensure even coverage of all samples being sequence in the run. Due to the high number of samples that are processed together in batch and the broad range of RNA input concentrations across samples entering the RT-PCR reaction, it is common to see high sample-to-sample variance in the DNA concentrations going into tagmentation and barcoding, and thus the number of reads for each sequencing. In order to explore the possibility of more uniform read depth for such a high batch of samples processed at a time, we tested manual normalization of A1200 amplicons after A1200 RT-PCR and barcoding PCR.

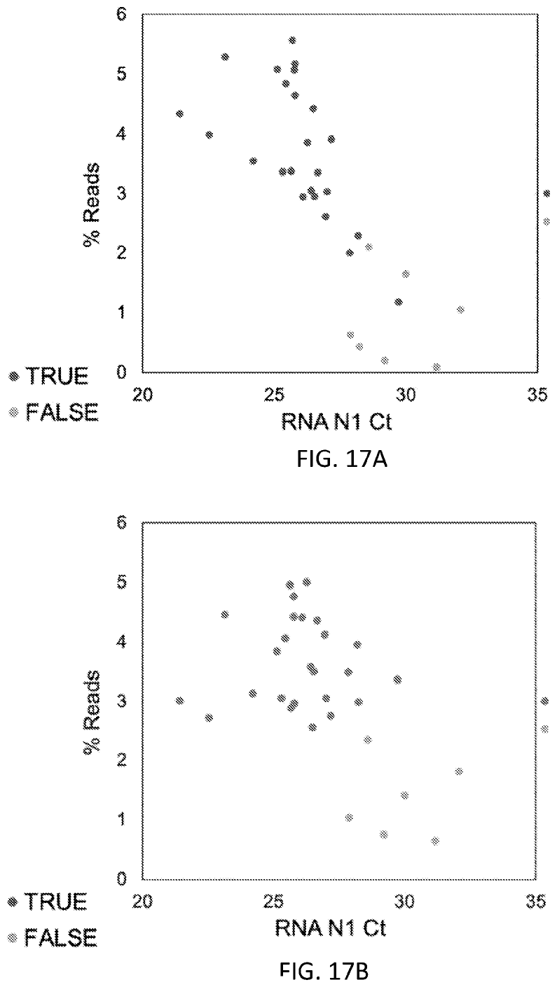

For amplicon normalization, the DNA concentrations of A1200 amplicons RT-PCRed from 32 RNA samples in the N1 Ct range of 20 to 32 were measured using a Qubit fluorescence assay. Following amplicon quantitation, the samples were individually diluted to 0.5 ng/uL. The rest of the tagmentation and barcoding steps were carried out as normal. After barcoding PCR, library quantification was again performed to obtain the concentrations of barcoded libraries. Based on the measured concentrations, the libraries were made equimolar before library pooling. This normalization method helped achieve somewhat more uniform percentage reads per sample across a large Ct range. The effect of manual normalization on DNA libraries is shown in FIG. 17A and FIG. 17B. Sequencing read depth versus RNA N1 Ct (20<Ct<32), n=32 for FIG. 17A un-normalized libraries and FIG. 17B manually normalized libraries. (Blue=genome reconstructed, orange=genome failed to reconstruct).

However, due to the limited effectiveness and high labor investment of this intervention, manual normalization of individual samples was not implemented in our pipeline. Instead, a universal 60-fold dilution factor for A1200 amplicons was implemented, which brought all samples into the <0.5 ng/μL range necessary for tagmentation to proceed efficiently.

The low Ct samples from each plate are purified with DNA Clean & Concentrator-5 (Zymo Research). The pooled libraries were eluted in 50 μL nuclease-free water. DNA concentration of the sample was measured using Qubit 1× dsDNA HS Assay Kit and the Plate reader (BioTech Synergy H1), and the sample was further diluted to 4 nM. Library was denatured and diluted according to the Denature and Dilute Libraries Guide by Illumina for MiSeq and NextSeq before being loaded on the Illumina sequencers following their screen instructions.

### Example 17: Hybrid Capture of High Ct Samples

Hybridization capture is a method for enriching select genome regions of interest from samples with high complexity or low concentration using antisense oligonucleotides to achieve better genome reconstruction with high sequencing coverage. This approach is highly useful for downstream applications, such as pathogen detection and identification, genomic characterization, and virulence determinants. We hypothesized that hybrid capture would be especially useful for improving the reconstruction rate of high-Ct samples, which did not perform as well in the A1200 RT-PCR reactions or downstream steps in the sequencing pipeline.

A hybrid capture technique was applied to improve genome reconstruction of more challenging samples with low viral loads using TWIST SARS-CoV-2 Research Panel (#103567, Twist Biosciences). Barcoded libraries from samples with Ct value above 26.5 from each sample plate were pooled together separately from the low Ct samples and then processed into a single hybrid capture reaction with a total of 0.7-1.5 ug DNA input at a time to enrich for SARS-CoV-2 sequences. Application of hybrid capture to high-Ct samples substantially improved the sequencing results from these samples.

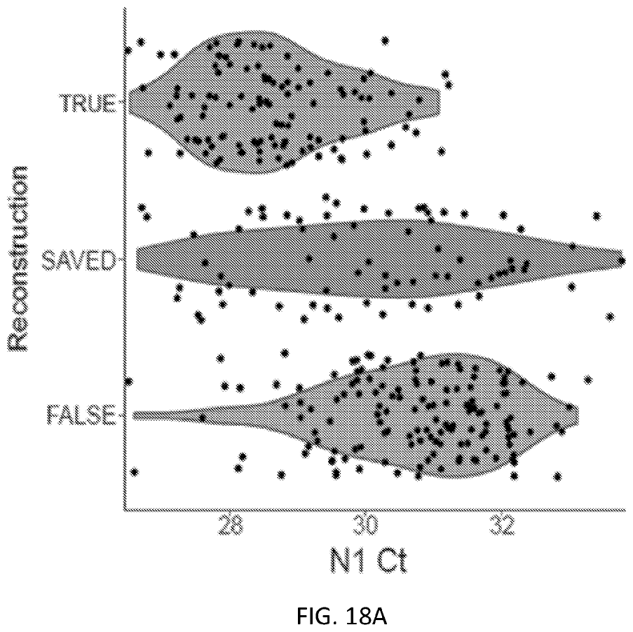

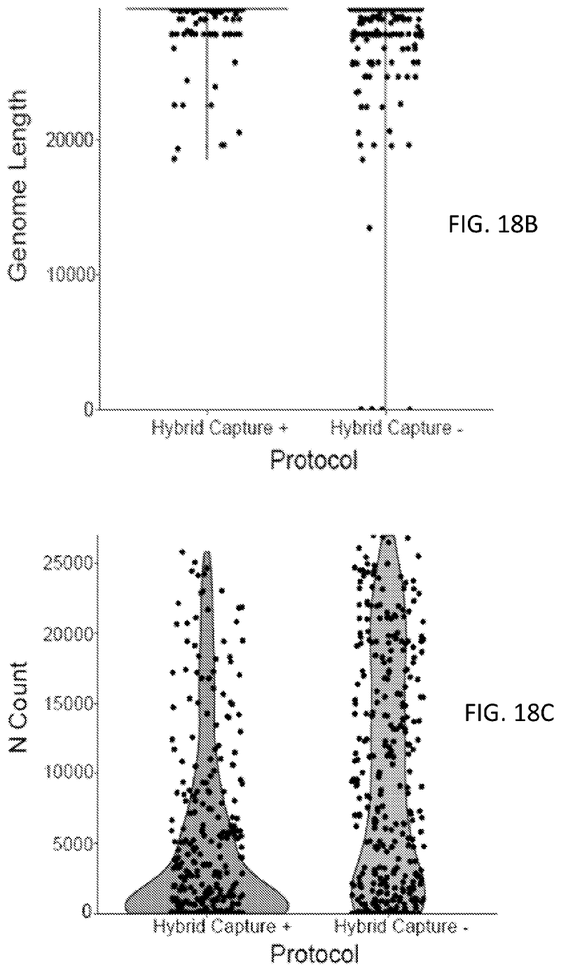

384 samples above Ct 26.5 were sequenced either with or without hybrid capture of barcoded libraries (FIG. 18A). Samples which were reconstructed in both conditions are labeled TRUE, samples which were reconstructed in neither are labeled FALSE, and samples reconstructed only when hybrid capture was applied are labeled SAVED. In accordance with APHL recommendation, a TRUE event is a consensus genome with greater than or equal to 90% breadth of coverage with less than or equal to 3000 ambiguous bases is considered a successful reconstruction. Saved samples are biased toward higher Ct value. Reconstructed genome length is plotted for both samples to which hybrid capture was and was not applied (FIG. 18B). Genome lengths are longer when samples are treated with hybrid capture. Uncalled bases are plotted for both samples to which hybrid capture was and was not applied. Samples have fewer uncalled bases on average if when hybrid capture is applied (FIG. 18C).

Of the 324 samples tested in the high Ct range of 26.5 to 33.5, 109 samples were observed which reconstructed without hybrid capture in comparison to the 181 samples that reconstructed with the application of hybrid capture with reconstruction rate of 56%—an improvement in reconstruction rate by 22% (FIG. 18A). We also observed a longer average genome length (FIG. 18B) and fewer uncalled bases (FIG. 18C) when hybrid capture was applied. Hybrid capture of high Ct samples achieved this by yielding both higher total reads and, more importantly, higher on-target reads for these samples.

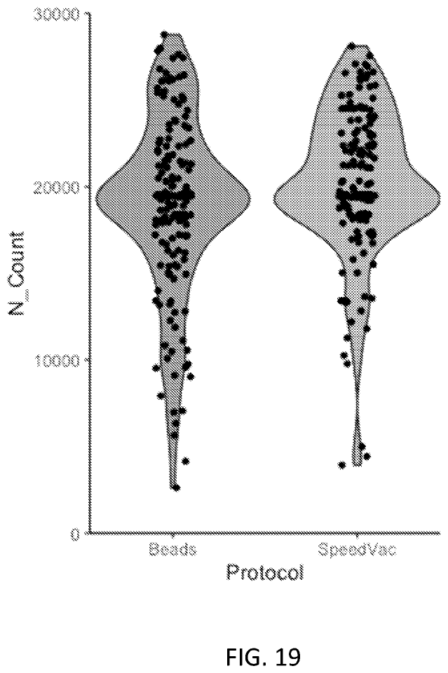

Finally, we were also able to improve the processing time of hybrid capture by testing the need of a dry-down procedure using SpeedVac as suggested by the Twist protocol. To assess the need for a SpeedVac step in the hybrid capture protocol, 330 high-Ct (>30) samples were tested with and without the SpeedVac step. No compromise in quality of the final products was observed by replacing the concentrating step with another bead purification step (FIG. 19). This step was omitted from the final pipeline to reduce labor.

For the high Ct samples, an extra process of hybrid capture is applied for target enrichment and better genome reconstruction. After the column purification, they are purified for the second time with 1× volume ratio of AMPure beads and eluted in de-ionized water (diH₂O). The rest of the process is carried out using the Twist Target Enrichment Workflow with minor adjustments to fit into our existing pipeline and the library construction method, and also to optimize for time and genome reconstruction efficiency.

TABLE 3
Genome reconstruction by low- and high-Ct value samples

| Sample | | genome_ | | | | | |
|---|---|---|---|---|---|---|---|
| number | N1 Ct | length | n_count | reconstruction | startpos | endpos | numreads |
| 1 | 36.61 | 28692 | 27240 | FALSE | 1 | 29870 | 636174 |
| 2 | 16.45 | 29763 | 0 | TRUE | 1 | 29870 | 70460153 |
| 3 | 36.78 | 27908 | 27110 | FALSE | 1 | 29870 | 94769 |
| 4 | 34.05 | 29745 | 28305 | FALSE | 1 | 29870 | 638308 |
| 5 | 29.58 | 29754 | 26013 | FALSE | 1 | 29870 | 1303975 |
| 6 | 40 | 29759 | 28458 | FALSE | 1 | 29870 | 284714 |
| 7 | 37 | 29741 | 28382 | FALSE | 1 | 29870 | 379046 |
| 8 | 35.97 | 28691 | 24311 | FALSE | 2 | 29870 | 1078926 |
| Sample | | | | | | | |
| number | % reads | covbases | coverage | average_coverage | meanbaseq | meanmapq | per_GeneLength |
| 1 | 0.849633499 | 28204 | 94.4225 | 507.253 | 34 | 42.5 | 96.05624372 |
| 2 | 94.10209521 | 29863 | 99.9766 | 174950 | 34.8 | 60 | 99.64178105 |
| 3 | 0.126567444 | 17562 | 58.7948 | 75.8203 | 33.9 | 39.7 | 93.43153666 |
| 4 | 0.852483533 | 10988 | 36.7861 | 582.205 | 34.2 | 42.2 | 99.58151992 |
| 5 | 1.741506006 | 15170 | 50.7867 | 2326.9 | 34.6 | 53.5 | 99.61165049 |
| 6 | 0.380245895 | 16630 | 55.6746 | 224.981 | 33.9 | 41.2 | 99.62838969 |
| 7 | 0.806229709 | 13782 | 46.1399 | 327.514 | 33.9 | 40.7 | 99.56812856 |
| 8 | 1.440944887 | 15596 | 52.2129 | 1801.53 | 34.6 | 51.5 | 96.05289588 |

### Example 18: Genome Assembly

For each specimen, sequencing adapters are first trimmed using Trim Galore v0.6.6 (Krueger, F. Trim Galore! A wrapper tool around Cutadapt and FastQC to consistently apply quality and adapter trimming to FastQ files. (2015)), then aligned to the SARS-CoV-2 Wuhan-Hu-1 reference genome (NCBI Nucleotide NC_045512.2) using BWA MEM 0.7.17-r1188 (Li, H. & Durbin, R. Fast and accurate short read alignment with Burrows-Wheeler transform. Bioinformatics 25, 1754-1760 (2009)). Reads that are unmapped or those that have secondary alignments are discarded from the alignment. Consensus and mutations were called using samtools and Intrahost variant analysis of replicates (iVar) (see, Grubaugh, N. D. et al. An amplicon-based sequencing framework for accurately measuring intrahost virus diversity using PrimalSeq and iVar. Genome Biol 20, 8(2019)) with a minimum quality score of 20, frequency threshold of 0.6 and a minimum read depth of 10× coverage. A consensus genome with ≥90% breath-of-coverage with ≤3000 ambiguous bases is considered a successful reconstruction (as per APHL recommendation). Variants were called using PANGOLIN v2.1.11 to v2.3.8 (see, O'Toole, A., McCrone, J. T. & Scher. E. Pangolin. (2020)).

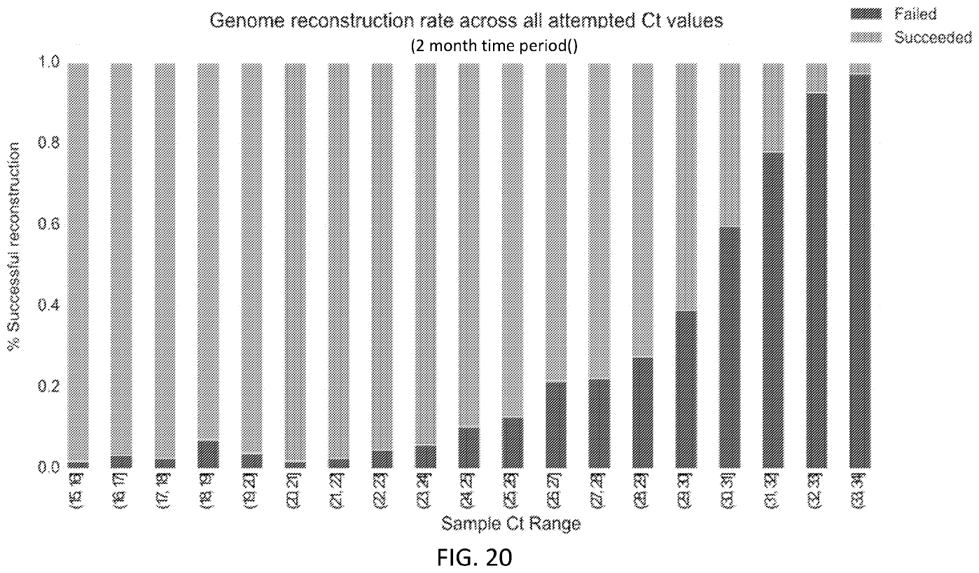

FIG. 20 shows SARS-CoV-2 genome reconstruction rates across all attempted Ct values over a 2 month period. The cumulative effect of all the pipeline optimizations described here allow genome reconstruction for samples above the Ct=30 threshold.

### Example 19: Assessment of Over-Sequencing of High Ct Samples as a Strategy to Improve Genome Reconstruction

Some example embodiments test whether an overload of sequencing reads to specific DNA libraries, by running only a few number of samples on the Illumina sequencer, would allow high Ct samples to be reconstructed. Samples with low RNA concentration (Ct≥30) often produce low-quality or incomplete genomes without targeted intervention to improve the results of these samples. We observed that high-Ct samples generally produce fewer sequencing reads than low-Ct samples Consequently, we wanted to test whether greatly increasing sequencing depth of these samples would allow high Ct samples to be reconstructed.

Seven samples with N1 Ct values between 29 and 40 (e.g., “high” Ct values) (wherein 40=Ct dropout) and a low Ct sample (N1 Ct=16.45) as positive control (Sample 2) were made equimolar and were loaded on the sequencer an Illumina NextSeq 500/550 Mid-Output v2.5 Kit (150 cycles). Even the number of reads (as high as 1,300,000 reads) for the high Ct value samples would have been expected to be sufficient for genome reconstruction, the samples nonetheless failed to reconstruct the genome. The results also showed a high number of undetermined bases (n_count) of 17,000 or above (out of 29,870), despite an average coverage that would be expected to be sufficient to reconstruct the genome in all cases (Table 3). This suggests that the quality of the source RNA in terms of sample degradation and concentration may be more crucial in deciding whether a sample reconstructs compared to the sequencing depth given to each sample.

### Example 20: Hitpicking Standard Operating Procedure (OT-2)

The purpose of this protocol is for reformatting positive RNA samples into Echo 384-well plate (e.g., LabCyte Echo 384-well plate) with a machine such as OT-2. In some embodiments, three controls are included on 384-well plate for quality control.

### Quality Control:

Inclusion of 3 control spots on well plate:
(1) Negative control: 40 μl of nuclease-free water in well P24
(2) Two positive controls: 40 μl of Positive Twist-RNA in both well B02 and well P22

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to the lab bench.

All sharps that have been exposed to biological materials, such as pipettes and Eppendorf tips, should be discarded into the sharps container next to the lab bench.

All materials that have not been exposed to biological material, such as pipette covers and packaging, should be discarded into the normal trash can located next to the lab bench.

### Equipment—Hardware & Software

(1) OT-2 Robot
(2) Hitpicking Source File

### Materials, Reagents and Forms

Materials
384-well Labcyte Echo plate
200 μl Filter Tip Racks, Opentrons Tips
Reagents
Eliminase
70% Ethanol
Nuclease Free Water
Twist-RNA Positive Control
Forms
N/A

### Procedure

### Before Starting:

1. Identify needed positive RNA source plates (sorted by the Line PCR Team) and scan the barcodes into Sequencing>Production>NYC-DOHMH>RNAplatesforPicking.

2. Given this list of total plates, the Data Science Team will provide the Hitpicking File—containing all positive samples from these plates (can be automated). The file can be available (in a few minutes) and added to Sequencing>Production>Software>Hitpicking>Output>Client>(date).

3. Clean and decontaminate the workbench using Eliminase and 70% Ethanol in preparation for the RNA plates and consumables.

4. Of all the plates to be processed, remove the first 9 plates (from −80 C storage) and begin thawing on ice.

### Prep

1. On the online shared drive, open Sequencing>Production>Software>Hitpicking>Output>Client>(date) and select a file for Hitpicking. Download this file onto the computer connected to the OT-2.
File name example: SEQ2021_01_29_0000.py

NOTE: Verify Wells B02, P22, and P24 are empty. These wells are reserved for positive and NTC controls.

2. Open the OT-2 program and click on Protocols on the left-hand menu and open the downloaded file. Click on “Proceed to calibrate.”

3. Calibrate OT-2.

4. Place the calibration tips (96×200 μl tips) on slot 11.

5. Place a 96-well Eppendorf plate on slot 1 and a 384-well Labcyte Echo plate on the cold block on slot 10. These plates will be marked specifically for calibration. Select “Calibrate” and follow the on-screen instructions.

6. Once completed, remove the calibration plates.

NOTE: Calibration tips, 96-well Eppendorf plate, and 384-well Labcyte Echo plate are empty and can be re-used for future calibrations. DO NOT use these plates and tips for the actual Hitpicking run.

### Run

1. Clean OT-2 slots and waste bin using Eliminase, then spray and wipe down with 70% Ethanol.

2. Retrieve an aliquot of the Twist-RNA Positive control, and pipette it into well B02 of a clean and empty 384-well Labcyte Echo plate. This plate will be the destination plate for the Hitpicked RNA samples.

NOTE: Wells B02, P22, and P24 of the destination Labcyte Echo plate are reserved for control samples. DO NOT add patient samples to these wells.

3. Place the destination 384-well Labcyte Echo plate on the cold block in slot 9.

4. According to the summary sheet, load RNA plates from the back, starting with slot

10. These plates must be centrifuged immediately before picking.

NOTE: Technologists should NEVER replace plates nor alter the worklist. If a plate from the summary sheet seems to be missing, the space on the deck must be left empty.

5. Inspect the plates to ensure that they are properly placed on the deck. The plates must be within the guidelines set for each slot or else the pipette may crash or aspirate from the incorrect well.

6. Select “Run.”

7. The OT-2 will pause after picking from each set of nine plates. Check that each well of the destination plate is full. If there are empty wells, manually add the entire volume of any missing samples from the appropriate source plate.

8. Discard the picked source plates into a biohazard bin and load the next set of RNA source plates, again starting from the back row.

NOTE: The OT-2 will prompt you to change the box of tips when necessary. At this point, please manually check the Hitpick summary file to see if it is time to insert the next set of RNA plates before pressing resume.

### End of Protocol

1. Once the protocol is complete, check the destination 384-well Labcyte Echo plate to ensure all wells from protocol are filled; note any wells that are empty or have low volume on Sequencing>Production>SequencingPipelineTracking.

2. Wells P22, and P24 will be empty as they are reserved for controls. Transfer the 384-well plate from cold block onto ice and pipette 40 μL of Nuclease-Free H2O into well P24.

3. Retrieve one aliquot of the Twist-RNA Positive Control from −80 C and pipette 40 L into well P22.

4. Seal the destination plate with adhesive foil. Label the plate using the date corresponding to the Hitpick protocol (if different than today's date).

5. Place the completed plate in the −80° C. freezer.

6. Update the Sequencing>Production>SequencingPipelineTracking with the plate location and hitpick date.

7. Notify the dayshift point of contact of these plates and their storage location, to schedule delivery of these plates to the R&D Team. The plates will be delivered the following morning.

8. Notify the R&D team point of contact about the incoming number of Hitpicked plates.

9. Empty the biohazard waste container in the biohazard room in a cardboard box.

10. Clean OT-2 with Eliminase and 70% Ethanol.

### Troubleshooting

OT-2: The robots may not appear to be connected online. To locate all available robots press “Command, Shift, R” simultaneously and connect to the desired OT-2 robot.

Plate sealer located on benchtop can be used to de-seal plates. Place 96-well Eppendorf plate onto deck and press run. De-sealed plate will come out smoothly.

### Example 21: Hitpicking Standard Operating Procedure (Tecan)

The purpose of this protocol is for reformatting positive RNA samples into Echo 384-well plate (e.g., LabCyte Echo 384-well plate) with a machine such as Tecan. In some embodiments, three controls are included on 384-well plate for quality control.

### Quality Control:

Inclusion of 3 controls on 384 well plate:
(1) Negative control: 40 μl of nuclease-free water in well P24
(2) Two positive controls: 40 μl of Positive Twist-RNA in both well B02 and well P22

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to the lab bench.

All sharps that have been exposed to biological materials, such as pipettes and Eppendorf tips, should be discarded into the sharps container next to the lab bench.

All materials that have not been exposed to biological material, such as pipette covers and packaging, should be discarded into the normal trash can located next to the lab bench.

### Equipment—Hardware & Software

(1) Tecan
(2) Hitpicking Source File

### Materials, Reagents and Forms

| | Catalog No. | Supplier | Storage | |
|---|---|---|---|---|
| Materials | | | | |
| 384-well PP 2.0 Microplate, | PP-0200 | Labcyte | 25° | C. |
| Echo Qualified | | | | |
| Tecan LiHa 200 μl Disposable | 30184272 | Tecan | 25° | C. |
| Tips - Filtered, Pure, Hanging | | | | |
| Reagents | | | | |
| Eliminase | 1102 | Fox West | 25° | C. |
| | | Sales Inc. | | |
| 70% Ethanol | BP28184 | Fisher | 25° | C. |
| | | Bioreagents | | |
| Nuclease Free Water | AM9930 | Invitrogen | 4° | C. |
| Twist-RNA Positive Control | 102024 | Twist | −80° | C. |
| | | Bioscience | | |

| | | | |
|---|---|---|---|
| Forms | | | |
| Tecan Tracker Sheet | N/A | N/A | N/A |

### Procedure

### Storage of RNA Source Plates

1. After the dispensing-step in the Innolabs PCR suite, the RNA plates are resealed.

2. The plates are moved to a 4° C. fridge grouped as a stack of 4 according to the LC plate number that it is associated with for clinical covid qPCR testing.

3. Periodically through the shift, a PCR technologist will take out specific positive plates from the fridge and put them in the −80° C.
RNA source plates with positive samples Ct value 34 and under are moved into storage in a −80° C. freezer.
The RNA plates that contain only negative samples or positive samples with Ct value greater than 34 are discarded in RWD biohazardous waste containers.

### RNA Source Plate Scanning

1. Open the online spreadsheet titled “RNAPlatesforPicking” from the Sequencing drive (this online spreadsheet is also favorited on the Tecan Computer).

2. Create a new date tab on the bottom of the sheet (e.g., 2_13).

3. Scan plates starting from bottom to top and sort into stacks of 20, while marking the last RNA plate on the top of the stack in sequential order (e.g., stack 1, stack 2, etc.).

4. Place the first 15-20 stacks in the designated 2-8° C. hitpicking refrigerator to thaw.

5. Place the remaining stacks back into the −80° C. freezer.

6. Notify the Sequencing Software team that you have finished scanning plates by tagging @hitpicking-worklist in the #proj-sequencing-hitpicking slack channel.

### Organization of Worklists

1. The @hitpicking-worklist team will post a link to the worklists in the #proj-sequencing-hitpicking channel.

2. Open each worklist and write down the first and the last plate of each worklist.

3. With your list, find the starting and ending plate of each worklist and clearly separate the worklists.

4. Leave the worklists that you will complete same day in the 2-8° C. refrigerator and store the worklists that you will not be completing in the −80° C. freezer.

NOTE: Technologists should never replace plates nor alter the worklist. If a plate from the worklist seems to be missing, the space on the deck must be left empty.

### Power-Cycling of Liconic, Tecan, and Tecan Computer

1. Before the start of each hitpicking day, the Liconic, Tecan, and Tecan Computer needs to be power-cycled.

2. Turn off the Liconic.
a. The power switch of the Liconic is green and is located on the front of the Liconic.

3. Turn off the Tecan.
a. The power switch is located under the Tecan deck inside the right door behind the white box.

4. Turn off the computer.

5. Turn on the Liconic.

6. Turn on the Tecan.

7. Turn on the Tecan Computer.

NOTE: It is essential to power cycle in the above correct order. If the power cycling is not performed in the above order, the Tecan will not initialize and thus not run the worklists.

Uploading Worklist into Fluent Software.

1. Open the first worklist that you will complete via online spreadsheets.

2. Download as a CSV file.

3. Open file explorer, and ensure that your worklist is named correctly (iRNA120449 RNA120442 e 2022_0213_00.csv)
a. Ensure that there are not any spaces or duplicate names.

4. Drag the file from “Downloads” into the “Hitpicking” folder.

### Preparation of the Tecan/Destination Plate:

1. Clean the Tecan deck, CPAC, and tip trays with Eliminase and 70% Ethanol.
a. Ensure that the CPAC control unit is turned on and is at the correct temperature

2. Load new Tecan LiHa 200 μL Disposable Tips—Filtered into each of the tip trays and load onto the Tecan.

3. Clean the workbench and P200 pipette with Eliminase and 70% Ethanol.

4. Obtain a new Labcyte Echo 384 well plate (this will be the destination plate).

5. Retrieve one aliquot of positive Twist-RNA control from the −80° C., thaw, then pipette 40 μL into well B02 of the destination 384-well Labcyte Echo plate.

6. Place the destination plate (with A1 in the top left corner) onto the CPAC.

NOTE: Wells B02, P22, and P24 of the destination Labcyte Echo plate are reserved for control samples. Do not add patient samples to these wells.

### Runs:

1. Open the Tecan Fluent Software on the Tecan computer.

2. Select the method “Picklist_CherryPick_PRL_ManualPlateLoading” on the Tecan Tablet.

3. As the fluent software prompts you, type in the “name” of your destination plate, which is also the same name as the worklist (e.g., 2022_0213_00). Select continue.

4. The fluent software will now simulate through the worklist script, this will take 2-3 minutes.

5. Obtain 12 thawed plates from the current worklist and centrifuge for 1 minute at 1000 rpm.

6. As the Fluent software prompts you, unseal the RNA source plate, scan, and place on the deck where the yellow blinking light is alerting you.

7. Inspect the plates to ensure that each one is properly placed on the deck (A01 in the back left corner).

8. After scanning your last plate, close the Tecan door and click “Continue.”

9. The channels will pick up tips to begin aspiration.

10. Using the Hitpicking file provided, the Tecan will begin aspirating the selected wells from the first source plate, and dispense in the designated wells of the destination plate. The Tecan will continue this process for the remaining source plates on the instrument deck.

11. Once the robot is done aspirating the specific wells based on the hitpicking file, a message will appear alerting you to manually remove the RNA source plates that are currently on the deck. Discard the RNA source plates in the RDW Biohazardous waste bin.

12. Continue steps 5-8 until the protocol has finished.

NOTE: The plates must be within the guidelines set for each slot or else the pipette arm and tips may crash or aspirate from the incorrect well.

### End of Protocol:

1. Once the protocol is complete, check the destination plate to make sure all wells are filled; note any wells that are empty or have low volume in the Notes column of the weekly tracking sheet. Also update the hitpick date, and end time.

⇒PRL R&D>Sequencing>Production>Software>Hitpicking>Output>Tecan Tracking Sheet>(date)

2. Wells P22, and P24 will be empty as they are reserved for controls. Cover the destination plate with a plate cover, then remove it from the CPAC onto the benchtop.

3. Pipette 40 μL of nuclease-free H₂O into well P24.

4. Retrieve one aliquot of positive Twist-RNA control from the −80° C., allow to fully thaw, and pipette 40 μL into P22.

5. Seal the destination plate with an adhesive foil seal.

6. Write the name of the destination plate on the front of the plate.

7. Tape down the foil to ensure that it will not fall off during storage and transport.

8. Place the completed plate in a styrofoam box on dry ice and store in the −80° C. freezer.

9. Post the name of the worklists that you have completed in both the #proj-sequencing-hitpicking channel and #seq-rna-transport slack channels.

10. Empty the biohazard waste container in the biohazard room in a cardboard box.

11. Clean the Tecan deck with Eliminase, then 70% ethanol.

12. Post the names of the completed destination plates in the #proj-sequencing-hitpicking and #seq-ma-transport slack channels.

NOTE: For plates extracted using the FeliX in 96-Well format, the Tecan is expected to aspirate and dispense 40 μL.

### Troubleshooting

Before pressing “Continue” after scanning the destination plate, close the Tecan Fluent Door.

After scanning the final source plate and before pressing “Continue” on the touchpad, close the Tecan Fluent Door.

Opening the Tecan Fluent Door will act as an emergency stop.

If the tips are not fully ejected, the user can gently pull the tips off of the channels.

To deal with static charge of the tips, gently lift the black collar up and down and then click “retry.”

If the destination barcode does not match the name of the file, double check the worklist name and the destination barcode inside of the csv file.

If a source plate scanned does not match the content of the worklist, the scanned plate will be skipped.

No source plate can be scanned into the system more than once.

### Example 22: Standard Operating Procedure for Receiving STAT Samples

The purpose of this protocol is to describe the standard procedures to be followed when STAT samples are received for processing.

### Safety Precautions & Procedures

Personal Protective Equipment (PPE) required for this procedure: Lab coat and gloves

### Procedure

1. The person-in-charge reaches out to Customer Success Team (CS team) regarding specific specimens requested for sequencing due to potential vax breaker or reinfection. These samples were previously run through the PCR line. Original swabs can be available.

2. If swabs are not available, the CS team can log the information in online spreadsheet. The online spreadsheet board will then alert a data scientist. The data scientist will pull the data.

3. CS team reaches out to a Clinical Lab Accessioner to pull the specimen and place in a −80° C. freezer.

4. Once the specimen is pulled, the CS team notes the requisition number, Ct values and date expected to be sent on the Monday.com board.

5. CS team then reaches out to facilities team to set up transportation.

6. Right before the specimen is transported, Clinical Lab Accessioner places the specimen in a secure box with dry ice.

7. Specimen is then sent from Location 1 to Location 2.

8. Once the specimen arrives at Location 2, the person-in-charge and the CS team are notified.

### Example 23: Standard Operating Procedure for STAT Accessioning

The purpose of this protocol is to organize patient information for clients and to track individual samples throughout the pipeline.

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to lab bench.

All sharps that have been exposed to biological materials, such as pipettes and Eppendorf tips, should be discarded into the sharps container next to lab bench.

All materials that have not been exposed to biological material, such as pipette covers and packaging, should be discarded into the normal trash can located next to the lab bench.

### Equipment—Hardware & Software

(1) Barcode Scanner
(2) Heat Inactivation Oven
(3) Sample Rack

### Procedure

### Receiving

1. STAT samples can be delivered to the lab by couriers and placed in the −80° C. freezer every day. Any samples that come the day before, or before 10 AM on the day of, will be accessioned (e.g., samples that come to the lab on May 4th and before 10 AM on May 5th will be accessioned on May 5th).

2. Place all the samples being accessioned into a bin and transport to the bench.

3. Remove each sample from its bag and place into the sample rack to be prepared for reformatting.
a. Examine each bag carefully for reasons for rejection before opening (typically leaking samples).

4. Once all the samples have been placed in racks, label the racks with the appropriate sequencing ID for the samples they hold.
a. The sequencing ID for the samples being accessioned should always be using the date that the samples arrived. (The sequencing ID for samples being accessioned on May 5th for example will be “STAT_2021_0504_91” since most, if not all the samples were delivered the day before).

### Tracking and Accessioning

1. Update the “lab_id”, “samples_processed_”, “sample wells used,96 format”, and “accessioning_date” sections of the sequencing pipeline tracking spreadsheet.

2. Scan all the samples sequentially (from A01-A12, B1-B12 . . . ) into the STAT Sample Order Checklist Spreadsheet.
a. The barcodes are scanned in order of priority: 1. Fat barcode, 2. Skinny barcode, 3. QR code (A Fat barcode would always be scanned if available, and a QR barcode would only be scanned if neither a fat nor skinny barcode is available).

3. Daily Extraction/Weekly Extraction: If extractions are occurring on a daily basis, the samples will continue along the pipeline to inactivation. If the samples are being batched and extracted on a weekly basis, they will be stored for the day of extraction.
a. Daily Extraction: After all the samples are in the correct order in the STAT Samples Order Checklist, heat inactivate all the racks in the oven for sixty minutes.
b. Weekly Extraction: If the samples are going to be extracted on a weekly basis, they will be stored in the −80° C. freezer after being scanned into the STAT Sample Order Checklist and accessioned on online spreadsheet. In this case, the rack should also be labeled as “Not Inactivated” in order to ensure that proper inactivation occurs on extraction day.

### Accessioning on Online Spreadsheet

1. All samples are accessioned on the STAT Accessioning Board.

2. Complete the “Sequencing ID,” “Accessioner,” “Date Received,” “Date Accessioned,” “Barcode,” “Client,” and “Status” columns.

3. The order of samples accessioned on the online spreadsheet should match the order of the samples on the corresponding rack.

### Maintenance and Storage

1. Keep the samples in the −80° C. freezer as often as possible to maintain sample integrity. Samples should only be outside of the −80° C. freezer if they are thawing or it is the day of extraction and they are being reformatted/extracted. Once a set of samples have undergone a freeze thaw, indicate on the rack label the amount of freeze thaws that has occurred.

2. Place all rejected samples in the rejection box in the back of the top shelf of the sample fridge.

3. Discard samples that have been in the freezer for more than two weeks.

### Reformatting Preparation (Weekly Extractions)

1. If a set of samples is to be reformatted the next day, the samples are to be thawed overnight at 3:55 PM the day before. (If a sample is being thawed on Wednesday, then they are to be thawed on Tuesday at 3:55 PM).

2. Heat Inactivation will occur the morning of sample reformatting in the case of weekly extractions. After heat inactivation, ensure that the rack label accurately reflects inactivation status.

### Trouble Shooting

1. Use the comments section on the online spreadsheet to indicate any special circumstances associated with a particular sample. Some of these instances may include having very low volume, or if it is a special request from a client.

2. Rejections: Samples that are leaking, have enough debris to prevent reformatting, or do not have enough volume to be reformatted should be rejected.
a. Samples that are rejected should still be given a sequencing ID that is placed at the very end of the plate (If the last sample that has been cleared to be accessioned is G5, all the rejections should be assigned slot G6 or above for that day).
   i. All samples that are rejected should be marked as “Failed” under the “Status” column.
   ii. If a sample has been rejected, a description should be given in the “Failure Reason” column.

3. Special Requests: There may be some instances where special request samples will be sent from a client. These samples typically require specific procedures that are outlined by the client.
a. In this case, the “Special Req|Req #” column should be completed with the requisition number provided with the special sample.
   i. If there is no requisition number provided, then the sample barcode should be filled into this column.
b. The location of the special requests on the plate may vary depending on the sample, but are typically assigned a sequencing ID at the end of the plate, but before the rejected samples.

### Example 24: Standard Operating Procedure for Reformatting

The purpose of this protocol is to accurately reformat samples from STAT sample tubes into 96-well plates.

### Quality Control:

Each 96-Well plate will have H1 serve as a negative control.

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to lab bench.

All sharps that have been exposed to biological materials, such as pipettes and Eppendorf tips, should be discarded into the sharps container next to lab bench.

All materials that have not been exposed to biological material, such as pipette covers and packaging, should be discarded into the normal trash can located next to the lab bench.

### Equipment—Hardware & Software

(1) P1000 Pipette
(2) P10 Multichannel Pipette
(3) Centrifuge
(4) Biosafety Cabinet
(5) 96-well plate
(6) Reservoir
(7) Proteinase K

### Procedure

### Proteinase K

1. In biosafety cabinet, pour 1000 μL of Proteinase K into a reservoir.

2. Use a P10 multichannel pipette to pipette 5 μL of Proteinase K into each well of the 96-well sample plate.

3. Thoroughly seal and roll the plate before removing from the biosafety cabinet and place in the centrifuge for 1000 rpm for one minute.

### Reformatting

1. Remove the proteinase K plate from the centrifuge and acquire a set of heat inactivated samples.

2. In the biosafety cabinet, unseal the proteinase K plate and orient the plate to prepare for reformatting.

3. One sample at a time, carefully uncap the sample and pipette 200 μL into the corresponding well in the 96 well plate. (A1 on the sample rack should go into A1 of the 96-well plate). Recap the sample and return to its designated slot on the sample plate.

4. Repeat this process until all corresponding wells are filled.

5. Ensure that any wells with rejected samples (should be labeled on sample rack) are skipped and that all other samples are in their designated wells.

6. H1 of the 96-well plate is also skipped as a negative control.

7. Seal the reformatted plate and leave in the biosafety cabinet while acquiring the necessary reagents for the first step of extraction.

8. After all the samples in the set have been successfully reformatted, navigate to the Sequencing Pipeline Tracking spreadsheet and fill in the “Reformatting Date” column.

### Trouble Shooting

1. Ensure that there is no contamination across samples and across each well. If a sample is pipetted into an incorrect well, discard the plate and restart the reformatting process.

2. Before beginning to reformat, ensure that the plate is in one of these desired orientations.
Vertically: A1 is on the bottom left-hand corner
Horizontally: A1 is on the top left-hand corner

3. When reformatting, take note of the viscosity of the sample being pipetted. Ensure that there are no bubbles in the tip and that the right volume is being transferred. Try to avoid mucus or any debris from entering the tip and being transferred to the 96-well plate.

### Example 25: Standard Operating Procedure for Extraction

The purpose of this protocol is to extract RNA from patient samples using KingFisher™ Flex (Thermo Scientific) instrument with the capability of extracting 96 samples in parallel.

### Quality Control:

This protocol can be used to extract 1-95 samples.

Each plate that is extracted must have an empty H01 well, serving as a negative control.

Once extraction is completed, quantitative PCR will be performed.

The extraction must be repeated if the negative control in well H01 produces C values<33 for N1,N2, or RP.

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to lab bench.

All sharps that have been exposed to biological materials, such as pipettes and Eppendorf tips, should be discarded into the sharps container next to lab bench.

All materials that have not been exposed to biological material, such as pipette covers and packaging, should be discarded into the normal trash can located next to the lab bench.

### Equipment—Hardware & Software

Thermo Scientific™ KingFisher™ Flex

### Materials, Reagents and Forms

| | | | Shorthand | | Storage |
|---|---|---|---|---|---|
| Materials | Catalog No. | Supplier | Notation | Storage | Location |
| 3 KingFisher ™ | 95040450 | Thermo | | 25° C. | |
| Deep Well 96 Plate | | Scientific | | | |
| 2 KingFisher ™ | 97002540 | Thermo | | 25° C. | |
| 96 Plate (200 uL) | | Scientific | | | |
| 1 KingFisher ™ | 97002534 | Thermo | | 25° C. | |
| 96 tip comb | | Scientific | | | |
| Trough | | | | | |
| Seals | | | | | |

| Reagents | Catalog No. | Supplier | | Storage |
|---|---|---|---|---|
| Binding Solution: 4M | | Teknova | Teknova | 25° C. |
| Guanidine Thiocyanate | | | Binding | |
| Buffer, 10 mM Tris-HCl, | | | Solution | |
| 1 mM EDTA, 20% PEG | | | | |
| 8000, 5% Tween-20 | | | | |
| SpeedBead Magnetic | | | SpeedBead | 4° C. |
| Carboxylate Modified | | | Magnetic | |
| Particles 100 mL, Azide | | | Particles | |
| 0.05% | | | | |
| Wash Solution: 10 mM | | Teknova | Teknova | 25° C. |
| TRIS, 1 mM EDTA, 2.5M | | | Wash | |
| Sodium Chloride, 20% | | | Solution | |
| PEG 8000, 0.05% Tween- | | | | |
| 20, pH 8.0 | | | | |
| Elution Solution: TE | | Teknova | TE Buffer | 25° C. |
| Buffer, pH 8.4 | | | | |
| Nuclease free water | AM9930 | Invitrogen | NF Water | 25° C. |
| Molecular grade 80% | T08204-K7 | Thermo | 80% EtOH | 25° C. |
| Ethanol | | Scientific | | |

### Reagent Plate Preparation

1. Binding Plate
a. For one 96 deep well plate, measure out 33 mL of the premade Teknova Binding Solution into a 50 mL Falcon tube.
b. Vortex SpeedBead Magnetic Particles bottle upright.
c. After vortexing, invert the SpeedBead Magnetic Particles bottle gently, and add 480 μL of the well mixed beads to the 50 mL Falcon Tube containing the Binding Solution.
d. Gently invert the 50 mL Falcon tube five times to ensure even distribution of beads then set on rocker for 5 minutes.
e. After 5 minutes of rocking, inspect the tube to ensure the solution is evenly mixed and appears homogeneous. If any beads appear to be clumping or collecting to one side, gently invert the tube 5-10 more times to ensure even distribution.
f. Pour this solution into a trough and using a P1000 multichannel pipette, add 300 μL to each well of the KingFisher™ 96 deep well plate, seal, and store at 25° C.

2. Wash Plate
a. For one 96 deep well plate, aliquot 53 mL of the Teknova Wash Solution into a trough.
b. Using a P1000 multichannel pipette, add 500 μL of this wash solution to each well of a KingFisher™ 96 deep well plate, seal, and store at 25° C.

3. 80% Ethanol Plate
a. For one 96 deep well plate, aliquot 53 mL of 80% Ethanol into a trough.
b. Using a P1000 multichannel pipette, add 500 μL of 80% ethanol to each well of a KingFisher™ 96 deep well plate, seal, and store at 25° C.

4. Elution Plate
a. For one 96 well plate, aliquot 5.3 mL of TE Buffer (pH 8.4) into a trough.
b. Using a P200 multichannel pipette, add 50 μL of TE Buffer into each well of a KingFisher™ 96 Well (200 uL) plate, seal, and store at 25° C.

NOTE: The Binding Solution and Beads Plates, Wash Plates, 80% Ethanol Plates, and Elution Plates can be prepared ahead of time and stored.

### Procedure

The reagents and plates necessary for an extraction are pre-prepared and stored at room temperature at the RNA extraction bench.

1. Obtain one of each of the following reagent plates and Tip Comb:
a. 1 Binding Plate (96 deep well KingFisher™ plate filled with binding solution and magnetic beads)
b. 1 Wash Plate (96 deep well KingFisher™ plate filled with wash solution)
c. 1 80% Ethanol Plate (96 deep well KingFisher™ plate filled with 80% EtOH)
d. 1 Elution Plate (96 well KingFisher™ 200 μL plate filled with elution solution)
e. 1 empty 96 well KingFisher™ 200 μL plate
f. 1 Tip Comb

2. Locate the Sample plate via the Sequencing Pipeline tracker.

3. Turn on the Biosafety Cabinet (BSC) (if turned off), and raise the front sash to the marked height. Ensure airflow is optimal, and the working light is turned on.

4. Disinfect the BSC work surface with Eliminase, followed by 70% Ethanol. Wipe down and allow to air dry completely.

5. In the BSC, with the P1000 multichannel pipette, transfer 200 μL of the patient sample/proteinase K into the binding plate. Pipette up-and-down 10 times to mix the binding/sample/proteinaseK.

6. Bring the binding/sample plate to the KingFisher™ Extraction bench.

7. Open the BindIt software on the KingFisher™ Flex computer.

8. Select the protocol “Bindtest1”

9. Click the green start button.

10. You will be prompted to name the protocol run, enter and save the run as the name of the sample plate that is being extracted.

11. Load the tip comb and reagent/sample plates in the order that the protocol instructs. Each plate should be oriented with A1 in the back left corner, matching up with the marking on the wheel “A1.”

12. After loading each individual tip comb/plate press the START button on the robot to advance to the next plate to load.

NOTE: Prior to placing the tip comb on the KingFisher™ Flex, rest the tip comb on the empty 96 well KingFisher™ 200 μL plate, this is the way that it will be loaded onto the robot.

13. Once the last reagent plate has been loaded and the start button has been pressed, the protocol will begin and run for 44 minutes.

NOTE: Watch until the tip comb has been picked up by the magnet comb to ensure that the run will begin successfully. Check the robot frequently to ensure the extraction is proceeding without error.

14. When the instrument protocol has completed, the KingFisher™ Flex will alert (beep), and the countdown clock on the computer will display 0s left.

15. Open the door of the instrument and take out the extracted RNA plate (elution plate), seal immediately, and place it in its designated bin in the 2-8° C. refrigerator.

16. After removing each individual plate, press start to advance the wheel to remove all plates and the tip comb.

17. Discard the binding, wash, and ethanol plates, and tip comb into the sharps container.

18. Wipe the wheel of the robot with a Kim wipe sprayed with 70% ethanol to clean.

19. Check the “run report” that the BindIt software generates to ensure that the extraction was completed successfully.

### Example 26: Standard Operating Procedure for RT-PCR

The purpose of this protocol is to amplify and detect RNA from the SARS-CoV-2 that target the nucleocapsid (N) gene and the human RNase P/RPP30 (RP) gene using reverse transcription quantitative PCR with SARS-CoV-2 primer and probe set.

### Quality Control:

Valid results from the amplification of an internal control targeting the human RNase P/RPP30 (RP) gene in a patient sample.

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to lab bench.

All sharps that have been exposed to biological materials, such as pipettes and Eppendorf tips, should be discarded into the sharps container next to lab bench.

All materials that have not been exposed to biological material, such as pipette covers and packaging, should be discarded into the normal trash can located next to the lab bench.

### Equipment—Hardware & Software

(1) Echo—Hardware & Software
(2) Lightcycler 480—Hardware & Software

### Materials, Reagents and Forms

| Reagents | Storage | |
|---|---|---|
| TAKARA | −20° | C. |
| nCoV_N1 Forward Primer | −20° | C. |
| nCoV_N1 Reverse Primer | −20° | C. |
| nCoV_N1 Probe | −20° | C. |
| nCoV_N2 Forward Primer | −20° | C. |
| nCoV_N2 Reverse Primer | −20° | C. |
| nCoV_N2 Probe | −20° | C. |
| RNase P Forward Primer | −20° | C. |
| RNase P Reverse Primer | −20° | C. |
| RNase P Probe | −20° | C. |
| Nuclease Free Water | 25° | C. |

### Procedure

1. Assemble Primer and Probe mixture in a 1.75 mL tube according to the table in “Calculations.”

2. Dispense 50 μL of the mixture in desired wells of a 384 Echo plate.

3. Dispense 5 μL of TAKARA into each well of a 384 well plate. Seal and spin at 2500 rpm for 1 minute.

4. Manually dispense 5 μL of RNA from sample plates into each respective well. Record the positions of each RNA plate to know where each sample is located on the 384 plate.

5. Seal and spin down the 384 well plate with the 5 μL TAKARA and RNA 5 μL.

### Echo

1. Spin down the Primer and Probe plate at 2500 rpm for 3 minutes.

2. Survey the Primer and Probe plate to ensure enough volume is in each well to shoot into the 384 well plate. The Echo can only fire volumes between 15 μL and 60 μL, so make sure the volume in each well is within this range.

3. Start a new protocol on the Labcyte Echo Plate Reformat software.

4. Select “Custom.”

5. On the Source Plate, select 4 consecutive wells that have sufficient volume of Primer and Probes.

6. On the Destination Plate, select the wells that need Primers and Probes.

7. Change the Volume (nL) to 150>Replicate Region.

8. Run protocol from the play button.

9. Seal plate with a clear seal and spin at 2500 rpm for 1 minute.

### Lightcycler 480

1. Select the Tree Diagram icon that leads to the home page.

2. Select “New Experiment form Template”.

3. Scroll down and select “PRL SCV2 N1 N2 Run Protocol.”

4. Select Detection Format “3 Color Hydrolysis Probe.”

5. Select 1 cycle for RT, 50 cycles for amplification, and 1 cycle for cooling.

6. Add 384 well plate with Takara, RNA, and Primers and Probes to the lightcycler.

7. Select start run.
a. Program runs for ˜1 hour.

### Analysis

1. When run is completed, select “Analysis.”

2. With each filter select “Calculate,” copy and paste data into an online spreadsheet.

3. Separate each 384 well plate into 4 quadrants. See Table 4 below.

### Stat

1. Select Online Drive>Production>NYC-HHC.

2. Create a folder of the date the samples processed.

3. Inside this folder, create an Online spreadsheet using the name of the plate.

4. Copy and paste the data from the separated quadrant file into each filter for N1, N2 and RP.

5. Select Online Drive>Production>NYC-HHC>Cumulative Genome Data

6. Scroll to the bottom of the sheet and copy and paste data to the sheet in sequential order.

7. Select Online Drive>Production>NYC-HHC>Cumulative qPCR Data.

8. Scroll to the bottom of the sheet and copy and paste data to the sheet in sequential order.

### Hitpick

1. Copy and paste the data from the separated quadrant file into each filter for N1, N2 and RP.

2. In the “qPCR QC/QA” Google Drive select the Python code named “qPCR_script.ipnyb.”

3. Follow the directions at the top of the page, making sure to change the text of “hitpicking_plate_barcode” to the name of the intended plate. Keep “hitpicking_quadrant” as 4. Change “qPCR_file_quadrant” to the quadrant of the samples of the intended plate. Change “qPCR_file_name” to the name of the file with all the raw data.

4. Runtime>Run All.

5. Check back in the Online Drive>Production>QC>Production QC-qPCR to make sure the file is in the folder and all R\hat{ }2 values are above 0.9, all graphs have a linear best fit line, and all controls are in expected ranges (B2 and P22 are positive controls and P24 is negative control).

### Calculations

| | One Echo | Two Echo | Four Echo |
|---|---|---|---|
| | Well | Wells | Wells |
| | Volumes (μL) of 100 μM Solution to Add | | |
| nCOV_N1-F (100 uM) | 10.5 | 21 | 42 |
| nCOV_N1-R (100 uM) | 10.5 | 21 | 42 |
| nCOV_N1-P (100 uM) | 2.625 | 5.25 | 10.5 |
| nCOV_N2-F (100 uM) | 6.75 | 13.5 | 27 |
| nCOV_N2-R (100 uM) | 6.75 | 13.5 | 27 |
| nCOV_N2-P (100 uM) | 1.6875 | 3.375 | 6.75 |
| RP-F (100 uM) | 3 | 6 | 12 |
| RP-R (100 uM) | 3 | 6 | 12 |
| RP-P (100 uM) | 0.75 | 1.5 | 3 |
| Nuclease Free H2O | 4.4375 | 8.875 | 17.75 |
| Total | 50 | 100 | 200 |

*NOTE:
Add Probes at the end, as they are sensitive to light.

### Trouble Shooting

### Exception Reports

1. From the Echo Exception report, copy the columns “Source Well,” “Destination Well,” and “Transfer Volume.”
a. If the Echo did not dispense any of the Transfer Volume, you can leave the column as is.
b. If the Echo dispensed some of the Transfer Volume but not all of it, you will need to subtract the volume it dispensed from the total transfer volume and use this volume as the number in the “Transfer Volume” column for the Exceptions Excel File you will be creating.

2. Open a new Excel File.

3. If there is more than one sheet in the Excel File, delete the ones you will not be using. Otherwise, Excel will not let you save it as a csv file.

4. Paste the Source Well, Destination Well, and Transfer Volume columns into the new Excel File.

5. Save the file as something you recognize in the folder “Echo exceptions” as a csv file.

6. Open the Echo Plate Reformat program. Open a new Echo file. Click the “Custom” option on the bottom of the pop up window. Then click “ok.”
a. For source plate: make sure “384PP” is selected as the plate type and “384_PP_AQ_BP” is selected as the liquid class.
b. For the destination plate: make sure “Roche384” is selected as the plate type.”

7. Go to File-->Import Region Definitions and click on the csv file you created with the source well, destination well, and transfer volume information you copied from the exceptions report.

8. Click ok.

9. The Echo will not let you dispense without saving the protocol. Save the protocol in the “Echo exceptions” folder as a name you will recognize.

10. Run the protocol as you would any other protocol.
a. To try and help prevent further exceptions, make sure there is enough volume from the wells you are trying to dispense from (between 15 and 60 l) and make sure the plate was spun down at 2500 rpm for 3 minutes.

### Hitpick Graph not Linear

1. Most common mistake is choosing data from an incorrect hitpick file or quadrant from qPCR plate.

2. Erase all data and restart from beginning.

### Example 27: Standard Operating Procedure for Tagmentation

The purpose of this protocol is to fragment A1200 amplicons from the previous RT-PCR step into 350-400 bp fragments, using the Tn5 Transposase, in preparation for barcoding PCR. The Tn5 Transposase uses enzymatic-based “shearing” and will simultaneously introduce P5 and P7 Illumina sequencing adapters.

### Quality Control:

Qiaxcel, E-Gels, or Fragment Analyzer may be used to ensure presence of 1200 bp band after RT-PCR. (A fragment analyzer or gel may be used again after tagmentation to check if DNA fragments are smaller, but not recommended since tagmentation reaction volume is only 1.5 l and all of it is needed as input for the barcoding reaction.)

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to the lab bench.

All sharps that have been exposed to biological materials, such as pipettes and Eppendorf tips, should be discarded into the sharps container next to the lab bench.

All materials that have not been exposed to biological material, such as pipette covers and packaging, should be discarded into the normal trash can located next to the lab bench.

### Equipment—Hardware & Software

(1) AnalyticJena FeliX
(2) Formulatrix Mantis
(3) 384-well thermocycler

### Materials, Reagents and Forms

| | Catalog No. | Supplier | Storage | |
|---|---|---|---|---|
| Materials | | | | |
| MANTIS Chip - Silicone, LV | 233581 | Formulatrix | °25 | C. |
| (0.1 μL & 0.5 μL), | | | | |
| RFID, PI. Pack of 6 | | | | |
| 384/60 μl or 96/60 μl | | AnalyticJena | °25 | C. |
| filtered CyBio RoboTip Trays | | | | |
| 384-deep well plate | | NEST or | °25 | C. |
| | | GBO | | |
| Armadillo 384 PCR plate | | Thermo | °25 | C. |
| | | Fisher | | |
| Reagents | | | | |
| Illumina Tagmentation Kit | 20034198 | Illumina | −20° | C. |
| Nuclease-free Water | | Invitrogen | 4° | C. |

### Procedure

1. Take Tagmentation DNA enzyme aliquot and Tagmentation buffer out of the freezer and let thaw on ice for about 15-20 minutes.

2. Make the required amount of Tagmentation Mastermix using Tn5 enzyme and Homebrew Tagmentation buffer (in a 1.5 mL Eppendorf Tube) according to the Table 5 below.

3. Keep the Tagmentation Mastermix on Ice until ready to use.

TABLE 5
Tagmentation Mastermix

| | | | 1 quadrant | | 2 quadrants | | Full 384 |
|---|---|---|---|---|---|---|---|
| | | | (110 | | (220 | | plate (384 |
| | 1 reaction | | Reactions) | | Reactions) | | reactions) |
| Tagmentation | 1.25 | μl | 137.5 | μl | 275 | μl | 550 μl |
| buffer | | | | | | | |
| Tn5 enzyme | 0.25 | μl | 27.5 | μl | 55 | μl | 110 μl |
| Tagmentation | 1.5 | μl | 165 | μl | 330 | μl | 660 μl |
| master mix | | | | | | | |

NOTE:
The above calculations account for an added 15% excess for 1 Quadrant, 2 Quadrants, and Full Plate calculations.
NOTE:
The Tagmentation Buffer is made in-house at a 1X concentration. The final concentration of the Tagmentation Buffer in the reaction is 0.5X.

4. Setting up the Formulatrix Mantis:
a. If the Mantis is turned off, turn it ON using the Power Switch located in the back of the instrument.
b. On the attached computer, launch the Mantis Control Software.
c. Before using each chip, a test dispense must be done to ensure accurate volumes are being dispensed. Load the required High Volume (HV) or Low Volume (LV) chip in the Chip Position and attach a P1000 tip with at least 100 μl of NF-Water.
d. Enter the desired volumes to be test dispensed, into the software, and designate where on the deck, the Mantis should dispense this volume.
e. After test dispensing, use a pipette to check the dispensed volume. If the dispensed volume matches the input volume, proceed to step “f” below.
f. Take the plastic deck off the center of the mantis and replace it with an aluminum 384 cold block (stored in the refrigerator).

NOTE: The Mantis nozzle height is adjusted to accommodate this cold block under the Armadillio 384 PCR plate.

5. Place a new empty 384-well PCR plate on the cold block (positioned on the Mantis deck).

6. Load up to 660 μL of the mastermix with a p1000 tip and use Mantis to dispense 1.5 μL into each well of a new 384-well PCR plate. This will be the “tag plate.”

7. Keep this plate on ice until ready to use.

8. In a new 384-deep well plate, using a p200 Multichannel pipette, dispense 120 μL of Nuclease-Free Water into each well. Label this plate “Dilution Plate.”

NOTE: Multichannel tips can be re-used as this is a new blank plate.

NOTE: For processing line samples (hitpicked at Location 1 with the Tecan), 120 μL water/well was used for samples with Ct≤28.5. Wells containing samples with Ct>28.5 will get 60 μL water/well.

9. Start “tag” protocol on thermocycler to preheat the block to 55° C.
a. Cycling conditions:
   (1) 55° C. PAUSE
   (2) 55° C. for 10 minutes
   (3) 10° C. HOLD

10. Setup Felix according to the diagram below.

| | | |
|---|---|---|
| Top deck of FeliX | | |
| position 7: P1 | position 8: | 9 |
| | Tag MMX plate | |
| | (384 Armadillo PCR plate) | |
| position 10: P2 | position 11: | 12 |
| | Dilution plate | |
| | (384 Deep-Well Plate - | |
| | contains 120 μL NF H2O) | |
| Bottom deck of FeliX | | |
| 1 | 2 | 3 |
| position 4: | 5 | 6 |
| 384 60 μl filter tips | | |

11. Launch the Felix control software and start “insert Felix protocol name here” protocol.

12. This Felix protocol will first mix the P1 and P2 RT-PCR products in the “Dilution plate,” and then transfer 1 μL of the diluted sample to the 384-well plate containing the Tagmentation Mastermix.

13. After the Felix protocol is completed, recover the 384-Well plate now containing the Tagmentation Mastermix and samples.

14. Seal with MicroSeal B Clear PCR Seal, and spin down at 2500 rpm for 30 seconds.

15. Transfer this plate to the thermocycler with the paused “Tag” protocol.

16. Press “start” to begin the tagmentation process. (Protocol name: tag)
a. Cycling conditions:
   (1) 55° C. PAUSE <<< should be held at 55° C. when loading plates
   (2) 55° C. for 10 minutes
   (3) 10° C. HOLD

NOTE: Samples may be held at 10° C. or stored on ice during the day, but must proceed to barcoding before the end of the day. Tagmented reactions should not be stored overnight without barcoding to avoid the possibility of over-fragmenting.

### Calculations

For tagmentation master mix:

| | | | | | | | Full 384 plate |
|---|---|---|---|---|---|---|---|
| | 1 reaction | | 1 quadrant | | 2 quadrants | | (440 reactions) |
| Tagmentation | 1.25 | μl | 137.5 | μl | 275 | μl | 550 μl |
| buffer | | | | | | | |
| Tn5 enzyme | 0.25 | μl | 27.5 | μl | 55 | μl | 110 μl |
| Tagmentation | 1.5 | μl | 165 | μl | 330 | μl | 660 μl |
| master mix | | | | | | | |

### Example 28: Standard Operating Procedure for Barcoding

The purpose of this protocol is to attach unique barcodes to samples before sequencing.

### Quality Control:

Negative control: Nuclease free water in well P24 of every plate.

Positive controls: Dilution of Twist Synthetic SARS-COV-2 RNA in wells B2, P22.

Echo reports are checked after each dispense to ensure the Echo dispensed correctly and to re-dispense any wells the Echo missed.

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to the lab bench.

All sharps that have been exposed to biological materials, such as pipettes and Eppendorf tips, should be discarded into the sharps container next to the lab bench.

All materials that have not been exposed to biological material, such as pipette covers and packaging, should be discarded into the normal trash can located next to the lab bench.

### Equipment—Hardware & Software

(1) Mantis—Hardware & Software
(2) Echo—Hardware & Software

### Materials, Reagents and Forms

| | Catalog No. | Supplier | Storage | |
|---|---|---|---|---|
| Materials | | | | |
| MANTIS Chip - Silicone, HV | 233580 | Formulatrix | 25° | C. |
| (1 μL & 5 μL), | | | | |
| RFID, PI. Pack of 6 | | | | |
| MANTIS Chip - Silicone, LV | 233581 | Formulatrix | 25° | C. |
| (0.1 μL & 0.5 μL), | | | | |
| RFID, PI. Pack of 6 | | | | |
| Reagents | | | | |
| Barcodes | | | 4° | C. |
| KAPA HiFi HotStart ReadyMix | KK2602 | Kapa | −20° | C. |
| | | Biosystems | | |
| Nuclease Free Water | | Invitrogen | 25° | C. |

### Procedure

### Mantis

1. Remove Kapa HiFi from the −20° C. freezer and thaw on ice.

2. Assemble the mastermix of Kapa HiFi and water in a 5 ml tube according to the table under “Calculations.”

3. Take out the 384 well cold block and place on ice.

4. For dispensing, make sure to choose “384_on_coldblock” as the plate type.

5. The mantis chips should be tested for the correct volume before use. For the Mantis HV chip, set the prime and pre-dispense volumes to 10 μL and 15 μL, respectively. For the Mantis LV chip, set both the prime and pre-dispense to 5 μL.

6. Load the tips with some water and have the chips dispense 3 different spots. For the HV chip, dispense 7 μL and for the LV chip, dispense 0.3 μL.

7. Use a pipette tip to measure the volumes of the dispenses to make sure the chips are dispensing correctly. If the volumes are correct, continue with the protocol. If not, try to dispense again. If the volumes are still off, discard the chip and use a new one.

8. Place your sample plate on the cold block and on the Mantis Deck. Load 730 μL into the tip of a p1000 and dispense 7 μL of Kapa Hifi master mix into each well, but dispensing a quarter of the plate at a time. Then, load the LV chip with 140 μL of master mix, set prime and pre-dispense both to 5 μL, and use the Mantis LV chip to dispense 0.3 μL to the entire plate.

NOTE: Make sure you do not place the cold block on the clear mantis stage, it should go directly on the mantis.

9. Seal the plate and spin down.

10. Put the cold block back on ice to let it get cold for the next sample plate.

11. When the chips are done for the day, clean the chips by setting the pre-dispense on each to 500 μL and load them with water. When the chips are done, rinse them with NF—H2O water bottle in the hood and store with the top (dispensing part) face up, in its labelled petri dish.

### Echo

1. Remove n5xx and n7xx barcoding plates from the 4° C. fridge and spin down at 2500 rpm for 3 minutes.

2. Open the “Echo plate reformat” program. The barcoding protocols are in the “0 SEQ STAT Production” Folder.

3. Use the Echo to ping 100 nL of both 10 μM n5xx and 10 μM n7xx barcodes. Make note of the barcoding strategy on the Sequencing Pipeline Tracker and on the plate itself (write the n5xx strategy used). Each plate will get the “384PP_n7xx_to_384PCRall” but a different “384PP_n5xx” protocol.
a. Make sure to use the appropriate barcodes per plate. If plates are going on the same sequencing run, they should get different n5xx barcodes.
b. Make sure to use the n7xx plate for the “384PP_n7xx_to_384PCRall” protocol and the n5xx plate for the “384PP_n5xx” protocol.

4. Seal the plate with a clear Thermo Fisher seal and spin it down. Insert the plate with a metal lid on it into the thermocycler. Run the “tagamp” protocol.
a. Cycling conditions:
   (1) 72° C. for 5 minutes
   (2) 98° C. for 5 minutes
   (3) 98° C. for 10 seconds
   (4) 61° C. for 30 seconds
   (5) 72° C. for 30 seconds
   (6) Repeat steps (3)-(5) for a total of 13 cycles
   (7) 72° C. for 5 minutes
   (8) 10° C. HOLD

### Calculations

For assembling Mastermix (2.5 μM combinatorial barcodes):

| | | | | | One 384-well plate | |
|---|---|---|---|---|---|---|
| | 1 quadrant | | 2 quadrants | | (440 reactions) | |
| 2x Kapa HiFi | 550 | μl | 1100 | μl | 2200 | μl |
| Water | 247.5 | μl | 495 | μl | 990 | μl |
| Total | 797.5 | μl | 1595 | μl | 3190 | μl |

### Volume Per Reaction:

| | Volume (μL) |
|---|---|
| 2x Kapa HiFi | 5 |
| Water | 2.25 |
| 2.5 μM n5xx barcode | 0.100 |
| 2.5 μM n5xx barcode | 0.100 |
| Tagmented input | 2.5 |
| Total | 9.95 |

For assembling Mastermix (2.5 μM Unique Dual Indexes or UDI barcodes):

| | Volume (μL) |
|---|---|
| 2x Kapa HiFi | 5 |
| Water | 1.65 |
| 2.5 μM UDI barcode | 0.800 |
| Tagmented input | 2.5 |
| Total | 9.95 |

### Trouble Shooting

1. From the Echo Exception report, copy the columns “Source Well,” “Destination Well,” and “Transfer Volume.
a. If the Echo did not dispense any of the Transfer Volume, you can leave the column as is.
b. If the Echo dispensed some of the Transfer Volume but not all of it, you will need to subtract the volume it dispensed from the total transfer volume and use this volume as the number in the “Transfer Volume” column for the Exceptions Excel File you will be creating.

2. Open a new Excel File.

3. If there is more than one sheet in the Excel File, delete the ones you will not be using. Otherwise, Excel will not let you save it as a csv file.

4. Paste the Source Well, Destination Well, and Transfer Volume columns into the new Excel File.

5. Save the file as something you recognize in the folder “Echo exceptions” as a csv file.

6. Open the Echo Plate Reformat program. Open a new Echo file. Click the “Custom” option on the bottom of the pop up window. Then click “ok.”
a. For source plate: make sure “384PP” is selected as the plate type and “384_PP_AQ_BP” is selected as the liquid class.
b. For the destination plate: make sure “Roche384” is selected as the plate type”

7. Go to File->Import Region Definitions and click on the csv file you created with the source well, destination well, and transfer volume information you copied from the exceptions report.

8. Click ok.

9. The Echo will not let you dispense without saving the protocol. Save the protocol in the “Echo exceptions” folder as a name you will recognize.

10. Run the protocol as you would any other protocol.
a. To try and help prevent further exceptions, make sure there is enough volume from the wells you are trying to dispense from (between 15 and 60 μL) and make sure the plate was spun down at 2500 rpm for 3 minutes.

### Example 29: Standard Operating Procedure for Sequencing

The purpose of this protocol is to pool individual barcoded samples together, and then prepare the library for sequencing.

### Quality Control:

Measure amount of pooled library, it should be 2× more than the number of samples being loaded onto the sequencer. If less, one should restart pooling. Cleaned libraries are also measured via Qubit (or qPCR) to make sure the correct ratio and concentration of libraries are loaded.

### Safety Precautions & Procedures

Personal Protective Equipment (PPE) required for this procedure: Lab coat and gloves

### Waste Disposal:

All materials that have been exposed to biological material, such as conical tubes, used tissue and gloves, should be discarded into the biohazard trash can next to the lab bench.

All sharps that have been exposed to biological materials, such as pipettes and Eppendorf tips, should be discarded into the sharps container next to the lab bench.

All materials that have not been exposed to biological material, such as pipette covers and packaging, should be discarded into the normal trash can located next to the lab bench.

The purged reagents contain formamide and need to be disposed of in a clearly labeled non-hazardous formamide container.

A small amount of formamide is left over in NextSeq cartridge post-sequencing run. Pour out any remaining liquid into the formamide receptacle and dispose of the used cartridge in the biological waste bin.

### Equipment—Hardware & Software

(1) MiSeq or NextSeq 500/500/2000
(2) BioTek plate reader
(3) Mini-centrifuge
(4) p-10 multichannel pipette

### Materials, Reagents and Forms

### Procedure

Important: Before beginning to prepare your library for sequencing by diluting and denaturing it, you must make sure that all Illumina consumables are available and being prepared for sequencing.

Before beginning:

1. Have a sample sheet generated specific to the barcoding primers used to generate the library.

2. Remove the appropriate sequencing cartridge 1 hour before running the sequencer. Place the cartridge in an autoclave bin or other large vessel and add water up to less than half the height of the cartridge to thaw the reagents (maximum to the notch at one corner of the cartridge).
a. Alternatively, the reagent cartridge can be thawed overnight at 4° C. The cartridge will be OK to use for up to one week after being thawed this way.

3. Also remove an appropriate flow cell from the 4° C. and allow it to come up to room temperature.

Note: After thawing in a water bath for 1 hour, place both the reagent cartridge and flow cell back at 4° C. or keep on ice.

Pooling the library

1. Remove the plate of barcoding reactions from the thermal cycler and briefly spin down.

2. Set up a 12-strip in a PCR rack and place on ice along with plates containing all reactions to be pooled.

3. Using a 12-channel P10 multichannel pipette, draw up 2 μL of each sample and transfer to the 12-strip. It is not necessary to change pipette tips between samples, since all samples are already barcoded.

4. When 2 μL of each sample has been transferred to the 12-strip, draw up all volume from each tube of the 12-strip and transfer to a single 1.5 mL centrifuge tube. The centrifuge tube should be labeled “Pooled LIBXXXX”, where XXXX corresponds to the next unused value of the library_id column of the Sequencing Pipeline Tracking spreadsheet.

5. Transfer 100 μL of the pooled library to a fresh 1.5 mL centrifuge tube. Use a Zymo PCR Clean and Concentrator tube to clean up the pooled library.
a. Zymo Kit instructions
   i. Add 1:4 ratio of sample to buffer, mix well in an Eppendorf tube. Transfer to a spin column.
   ii. Centrifuge for 30 seconds at 16,000 G.
   iii. Remove supernatant.
   iv. First wash: Add 200 μl of wash buffer, spin for 30 seconds at 16,000 G
   v. Remove supernatant.
   vi. Second wash: Add 200 μl of wash buffer, spin for 30 seconds at 16,000 G.
   vii. Dry: Spin for 2 minutes at 21,000 G. This step removes any residual ethanol left in the column.
   viii. Elution: Add 50 μL of Zymo kit buffer directly to the column and let sit at room temperature for at least one minute. This gives the DNA enough time to solubilize. Insert the column into a fresh 1.5 mL centrifuge tube labeled “Clean LIBXXXX.” Spin down at 10,000 G for 1 minute to elute the DNA. This library is now clean and ready to be prepared for loading on the sequencer.

6. Measure the DNA concentration using the Qubit Master Mix following the Library Quantification (qPCR and Qubit) Protocol. The expected DNA concentration is generally around 30-80 ng/μL based on the current protocol, but may vary a good deal in this range. Values under 10 ng/μL are suspect based on the current protocol. The Basic Qubit Spreadsheet will automatically calculate the molarity of your sample based on an average fragment size of 300 bp, and the required volume to dilute 5 μL of your library to 4 nanomolar. Instructions are in the “Calculations” section.

### Qubit

For the protocol, prepare:

1. The library to measure.

2. Qubit standards 1 (0 ng/μL) and 2 (10 ng/μL).

3. Black 364 well plate.

Assemble the standard curve:

1. Leave the leftmost well of a single row empty. In the next 7-11 adjacent wells in the row, pipette 10 μL of Qubit Standard 1. These volumes will be used for serial dilution.

2. In the leftmost well, pipette 10 μL of Standard 2. This well now contains 100 ng of DNA.

3. Draw up another 10 μL of Standard 2 from the bottle. Pipette into well 2 of the row and pipette up and down to mix. Discard this pipette tip.

4. With a new tip, draw up 10 μL of volume from well 2 and transfer it to well 3. Pipette up and down to mix. Proceed in this way, serially diluting the sample using a new tip each time to ensure accuracy of the standard curve.

5. When you reach the second to last well of the dilution series, draw up 10 μL of this mix and discard the tip without transferring the volume to the next well. The rightmost well should contain 0 ng/μL of DNA.

6. Pipette 90 μL of Qubit Master Mix into each well of the standard curve, pipetting up and down to mix. Use a new pipette tip for each well.

7. Allow to sit several minutes before measuring.

Mix the Library with Qubit Master Mix

1. To a well to the right of the standard curve, add one microliter of the library to be measured. One microliter of each library is measured so that, when fitted to the standard curve, the value obtained is the ng/μL of the library. Pipette each library to be measured in its own well.

2. Add 99 μL of Qubit Master Mix to each library to be measured, and pipette up and down to mix.

3. Allow to sit for several minutes before measuring.

### Measure the Library

1. Take the plate to the plate reader and open the Gen5 3.10 software. Select “Create New . . . ” and choose Standard Protocol.

2. Click the “Procedure” Icon on the task bar.

3. Click “Read” and choose Detection Method: Fluorescence Intensity. Click OK, and click OK again to accept the default excitation and emission wavelengths of 485 and 528 nm.

4. On the Procedure window, choose Plate Type: 384 WELL PLATE, deselect “Use Lid”, and choose Select wells: At runtime.

5. Click OK.

6. Click the “Create Experiment and Read Now” Icon on the task bar.

7. Select the wells containing the standard curve and samples and click OK.

8. The plate reader will open. Place the plate with well A1 oriented toward the back right corner of the machine.

9. Click OK to start the run.

10. After the plate runs, the software will prompt you to save your protocol. This is optional.

11. Select the wells containing the data to be exported.

12. Right click and choose “Copy to Clipboard.”

13. Here, you can paste your data into the Basic Qubit Spreadsheet to quickly calculate the concentration of your sample.

Loading the sequencer

1. When your cartridge is thawed, invert several times to mix buffers. Tap against the bench top to make sure all buffers are settled.

2. Press the “Sequence” button on the NextSeq.

3. Perform the run manually (do not use Local Run Manager) and use Basespace for data storage and run monitoring. Select Next.

4. Log in to your account.

5. Choose the sequence hub and select Next.

6. Enter a run name including the date and the sequencing platform (e.g. 210201_nextseq).

7. Enter the library ID (e.g. LIB0015).

8. Select the read type, “paired-end.”

9. Select the read length (76) and index length (8).

10. Select the appropriate sample sheet. This should be placed in the sample sheets folder on the Desktop. Be sure the date is correct in the name of the sample sheet and in the body of the sample sheet as well.

11. Select Next.

12. The door to the flow cell should automatically open at this time. Remove the old flow cell. Remove the new flow cell from its packaging and wipe the surface clean first with an alcohol pad, then dry Kim wipe. Make sure there aren't any smudges or lint on the surface. Load the new flow cell and click Load.

13. Replace the buffer container and empty old buffer waste.

14. You'll be prompted to empty the spent reagents container. Remove the container from the NextSeq, open the valve, and empty it into the formamide waste container below the sequencers. Load the spent reagents container back into the machine.

15. Remove the old buffer cartridge and throw it in the biohazard waste container. Open a new buffer cartridge and load it on the machine. Close the door and click Next.

16. Open the door on the left of the machine. Remove the old cartridge. Press down on the clear plastic tab and push towards the left to eject the reservoir. Empty this in the formamide waste. Dispose of the cartridge and the reservoir in the biohazard waste.

17. Pierce the foil on the appropriate well and follow the instructions in the denature and dilute libraries guide to load the correct volume of your sample into the well.

18. Load the new cartridge into the NextSeq.

19. Close the door and click Load.

20. Click Next.

21. When everything is loaded, review the parameters of the run. When you are satisfied everything is correct, begin the run.

### Calculations

### Convert Your Sample to nM

1. Go to the NEBio Calculator (https://nebiocalculator.neb.com/#!/dsdnaamt). Choose Mass-->Moles.

2. Enter a DNA length of 300 bp. This should be the average size of the library. Enter the mass of your sample in ng/μL, and change the value of this to “mg” (ng/μL=mg/L). The NEBio calculator will now display the number of moles in the sample.

3. Since we have essentially converted our ng/μL to mg/L, this is the hypothetical number of moles in a liter of our sample. Therefore, our sample is at a concentration of 312.2 nanomolar.

### Dilute You Sample to 4 Nanomolar (nM)

1. Add 5 μL of your sample to a new tube labeled “4 nM LIBXXXX”. 5 μL is used to ensure accurate pipetting.

2. To calculate the volume to dilute with to achieve 4 nM use the following equation:

$$Volume = ( ( [ Sample conc ] / 4 nM ) * 5 µL ) - 5 µL$$

In this example, Volume=(([312.1]/4)*5)−5=385.1 μL. Or, just use the Basic Qubit Spreadsheet to automatically calculate this.

3. Add this volume of 10 mM Tris-HCl+0.05% Tween20 to your sample.

### Dilute and Denature Your Library

1. Proceed with library preparation as described in appropriate denature and dilute libraries guide, pages 4-6 for the NextSeq or the MiSeq. These can also be found in our online drive here.

2. The current SOP (06_08) for loading the NextSeq entails diluting to 1.49 pM for the NextSeq High output and 1.42 pM for the NextSeq mid output based on consistent overclustering when loading at the recommended concentrations. This entails combining 97 μL of denatured library to 1203 μL of HT1 buffer for the 1.49 pM high output library, or 92 μL of denatured library to 1208 μL of HT1 buffer for the 1.42 pM mid output library.

### Example 30: Barcoding Experiment

The purpose of this protocol is to identify ideal amount of barcoding primers to sue for sequencing.

1. Select one or two low Ct samples.

2. From amplicon dilution plate, tagment as normal.

3. Dispense Kapa hifi master mix (use usual amount of water per reaction, do not change to compensate for more or less primer used).

4. Shoot barcode primers with Echo.
a. Make a custom Echo protocol to only shoot into specified wells.

5. Repeat steps 2-4, but modify primer volume to 25 nL, 50 nL, 75 nL, 100 nL, and 125 nL.

### Example 31: UDI Barcoding Experiment (Using Patient Sample)

### Part I

### Day 1

1. Check concentration of positive control cDNA.
a. Day 1—combine control of p1 and p2 of hitpick RT-PCR and check concentration.
b. Figure out dilution volume (target concentration is 0.6 ng/μL).
c. Figure out how many samples needed to set up to get 1152 μL of cDNA input.

### RT-PCR

a. Using Twist control, set up 1-3 reactions (1 μL Twist control RNA+5 μL Takara)
b. Dilute 10\hat{ }6 stock Twist RNA to 10\hat{ }3 by mixing 5 μL of RNA with 4995 μL of nuclease free H₂O. Aliquot into strip tubes and store at −80° C.
c. Using the 10 mM A1200, make 8 RT-PCR reactions worth following:

| | | |
|---|---|---|
| Takara | 5 | μl |
| A1200 | 0.5 | μl |
| H₂O | 3.5 | μl |
| RNA | 1 | μl |
| Total Volume for one reaction: | 10 | μl |

d. Thoroughly mix all together in a 2 mL tube. Divide into 8 strip-tubes and transfer to a 96 well Eppendorf plate. Spin down and fun TAKARA GENOME protocol on thermocycler.

### Day 2

3. Qubit
a. Find concentration.
b. Dilute to 0.5 ng/μL (approximately 100 fold)
c. Combine cDNA of all four tubes (A, B, C, D) into one 5 mL tube.

4. Tagmentation
a. Make enough tagmentation master mix for 3 full tag plates.
b. Need at least 1152 μL of cDNA input+1728 μL of tag master mix.

5. Multichannel on top of three 384-well plates.

6. Dispense Kapa master mix with mantis or multichannel.

7. Barcode with UDI plates 1, 2, 3.

8. Pool and sequence.

### Part II

1. Make an Echo plate with specific amplicons (see pool 2 amplicon mix of 11 individuals, and three sets that will stay separate).
a. Pool 1: P11, P19, P23
   i. Choose these amplicons based on their representation on 2021_0720_91 stat run.
   ii. Goal: P2 primer scheme so each 384 well gets different combination of 5 different P2 amplicon->Echo protocol has information.
   iii. Each UDI pair plate gets this P2 combination+different P1 amplicon.
b. Pool 2: see above
   i. Need to dilute these 5-fold.

2. Take 5 μL of patient cDNA that has reconstructed TRUE by combining 30 samples from the dilution plate.
a. Need: 2 mL cDNA for full 384 well P2 plate and 1.5 mL cDNA for P1 plate.
b. Pool enough for 600 reactions, only need 528 reactions.

3. Mix 3 mL of pooled cDNA from amplicon dilution plate samples, mix with 3 mL Takara.

4. Each reaction got 10 μL of the cDNA+Takara mixture.

5. P2: Full 384 well plate.
a. P1: rows A+B for P11 amplicon, C+D for P19 amplicon, E+F for P23 amplicon
b. Run Takara RT-PCR.

6. Quantify with Qubit: if samples are too concentrated to read, dilute each 1:10.
a. P11: 22.3 ng/μL
b. P19: 36.9 ng/μL
c. P23: 42.6 ng/μL
d. Well A1 on P2 plate: 43.6 ng/μL
e. Well H1 on P2 plate: 31.2 ng/μL

7. Dilute P1 amplicons and P2 amplicons 1:10.
a. Each P1 amplicon on a different 384 PCR plate.

8. Combine 2 μL from P1, 2 μL from P2, and 120 μL H₂O for dilution for tagmentation.
a. Each UDI barcode pair get a different P1 amplicon plate, but the same P2 amplicon plate
b. 2021_0805_00_BC1: P11 amplicon+P2 plate+UDI barcode pair 1
c. 2021_0805_00_BC2: P19 amplicon+P2 plate+UDI barcode pair 2
d. 2021_0805_00_BC3: P23 amplicon+P2 plate+UDI barcode pair 3

### Example 32: UDI Barcoding Experiment (Using Positive Sample)

### Part I

### Day 1

1. RT-PCR
a. Using Twist control, set up a full 384 plate of reactions (1 μL Twist control RNA+8.5 μL Takara-MMX)
b. Dilute 10\hat{ }6 stock Twist RNA to 10\hat{ }4 by mixing 5 μL of RNA with 495 μL of nuclease free H₂O. Aliquot any excess into strip tubes of 40 μL and store at −80° C.
c. Take an aliquot of the amplicons listed below. Put 35 μL of said amplicon into appropriate wells of the echo plate.

| | | | for 440 reaction | |
|---|---|---|---|---|
| | for one reaction | | (for 384-well plate) | |
| Takara | 5 | μl | 2200 | μl |
| A1200 | 0.5 | μl | — | |
| H₂O | 3.5 | μl | 1540 | μl |
| RNA | 1 | μl | 440 | μl |
| Total Volume for one reaction: | 10 | μl | | |

d. Thoroughly mix the 10\hat{ }4 diluted Twist control/Takara/H₂O mix together in a 5 mL tube. Use a multichannel to add 9.5 μL into each well of a 384 well plate. Spin down and use the echo to shoot the amplicons using “UDITest_p2_ALL” (located in the documents folder on the Echo PC). Spin down and run TAKARA GENOME protocol on thermocycler.

### Day 2

2. Qubit
a. Use the Felix the mix 4 μL from the amplicon plate into 120 μL of an amplicon dilution plate.
   i. Dilute to 0.5 ng/μL (approximately 100 fold).
b. Use the Felix to dispense 1 μL from the amplicon dilution plate into the tagmentation plate.

3. Tagmentation
a. Make enough tagmentation master mix for 3 full tag plates.
b. Need at least 1152 μL of cDNA input+1728 μL of tag master mix.

4. Multichannel on top of three 384-well plates.

5. Dispense Kapa master mix with mantis or multichannel.

6. Barcode with UDI plates 1, 2, 3.

7. Pool and sequence.

### Part II

1. Make an Echo plate with specific amplicons (see pool 2 amplicon mix of 11 individuals, and three sets that will stay separate).
a. Pool 1: P11, P19, P23
   i. Choose amplicons based on their representation on 2021_0720_91 stat run.
   ii. Goal: P2 primer scheme so each 384 well gets different combination of 5 different P2 amplicon->Echo protocol has information.
   iii. Each UDI pair plate gets this P2 combination+different P1 amplicon.
b. Pool 2: see above.
   i. Need to dilute these 5-fold.

2. Take 5 μL of patient cDNA that has reconstructed TRUE by combining 30 samples from the dilution plate.
a. Need: 2 mL cDNA for full 384 well P2 plate and 1.5 mL cDNA for P1 plate.
b. Pool enough for 600 reactions, only need 528 reactions.

3. Mix 3 mL of pooled cDNA from amplicon dilution plate samples, mix with 3 mL Takara.

4. Each reaction got 10 μL of the cDNA+Takara mixture.

5. P2: Full 384 well plate.
a. P1: rows A+B for P11 amplicon, C+D for P19 amplicon, E+F for P23 amplicon.
b. Run Takara RT-PCR.

6. Quantify with Qubit: if samples are too concentrated to read, dilute each 1:10.
a. P11: 22.3 ng/μL
b. P19: 36.9 ng/μL
c. P23: 42.6 ng/μL
d. Well A1 on P2 plate: 43.6 ng/μL
e. Well H1 on P2 plate: 31.2 ng/μL

7. Dilute P1 amplicons and P2 amplicons 1:10.
a. Each P1 amplicon on a different 384 PCR plate.

8. Combine 2 μL from P1, 2 μL from P2, and 120 μL H₂O for dilution for tagmentation.
a. Each UDI barcode pair get a different P1 amplicon plate, but the same P2 amplicon plate.
b. 2021_0805_00_BC1: P11 amplicon+P2 plate+UDI barcode pair 1
c. 2021_0805_00_BC2: P19 amplicon+P2 plate+UDI barcode pair 2
d. 2021_0805_00_BC3: P23 amplicon+P2 plate+UDI barcode pair 3

### Example 33: Standard Operating Procedure for Sequencing

The purpose of this protocol is to describe the standard procedures sequencing.

### Loading

### Sequencing Consumables

| Consumable | Quantity | Storage | |
|---|---|---|---|
| Cartridge | 1 | −20° | C. |
| Flow cell | 1 | 4° | C. |
| RSB with Tween 20 | 1 | −20° | C. |

Note: The label on the cartridge indicates how many cycles are analyzed, not how many cycles are performed. All 100-cycle and 200-cycle cartridges include an extra 38 cycles. The flow cell is compatible with any number of cycles.

### Sample Sheet

1. Open sample sheet template “template_NextSeq2K”
a. “Save as” to rename file
b. Rename the file according to the run name—ex: 210731_NextSeq2k
c. Sample sheets should be saved in (insert sample sheet location)

2. Modify line 3 (run name)
a. Ex: 210731_NextSeq2k

3. Modify from line 21 and down

4. Open sample sheet builder “REVCOM_samplesheetbuilder_miseq/2k”
a. Use builder to align sample names with indexes based on well location
b. Copy
c. Paste into line 21
d. Remove index names, keep only sequences for index (i7) and index2 (i5)

5. Save and upload onto Basespace

### Thawing the Cartridge

Note: There is no easy way to determine if the cartridge is fully thawed; so it is important to follow these guidelines as well as plan ahead. Thawing times are long.

1. Remove cartridge from −20° C., make sure it has the correct number of cycles.

2. Recycle outer cardboard container.

3. Do not open the foil container. Keep in its foil container and thaw at room temperature (20° C. to 25° C.) for a minimum of 9-16 hours. Keep upright according to package instructions.

4. Transfer cartridge to 4° C. after thawing, cartridge is stable for 72 hours at 4° C.

5. Cartridges stored at 4° C. will need to be brought to room temperature for 15 minutes to 1 hour prior to run start.

### Preparing and Diluting the Library

1. (Steps for pooling samples)

2. (Steps for cleaning with Zymo kit or other method)

3. (Steps for quantifying library using Qubit 1s ds DNA kit and H1 plate reader)

4. Using RSB with Tween 20 as a diluent, dilute library to 2 nM. You will need at least 24 μL.

5. Vortex library briefly, then centrifuge at 280×g for 1 minute.

6. Dilute the library to loading concentration, this varies by library type: 650 μM loading concentration:

| 2 nM library (μL) | RSB with Tween 20 (μL) | Total Volume (μL) |
|---|---|---|
| 7.8 | 16.2 | 24 |

7. Vortex library briefly, then centrifuge at 280×g for 1 minute.

8. The loading volume is 20 μL.

9. Keep on ice until ready to load.

### Setting Up a Run on BaseSpace

1. Navigate to BaseSpace Sequence Hub.

2. Log in with email address and password.

3. Make sure to select ReopenLabs account, not your personal account.

4. Select “Runs.”

5. Select “New Runs.”

6. Select “Instrument run set up.”

7. It will bring you to a new screen to enter specific run information.

8. Select “NextSeq 1000/2000.”

9. Enter Run name.
a. This should match with the sample sheet
b. Ex: “210731 NextSeq2k”

10. Analysis location: BaseSpace.

11. Type of Analysis: Illumina DRAGEN FASTQ Generation—3.8.4.

12. Library PrepKit: Nextera XT DNA.

13. Index Reads: 2 Indexes

14. Read Type:

### Combinatorial

| Read 1 | Index 1 | Read 2 | Index 2 |
|---|---|---|---|
| 61 | 8 | 61 | 8 |

### UDI

| Read 1 | Index 1 | Read 2 | Index 2 |
|---|---|---|---|
| 59 | 10 | 59 | 10 |

### Loading Consumables

Note: Take flow cell and cartridge from 4° C. to room temp for 15 minutes before loading to avoid condensation.

1. Open foil cartridge package.

2. Invert 10 times to mix reagents. Internal components may rattle, this is normal.

3. Open the foil flow cell package.

4. Pull the flow cell out of the bag, keep the bag just in case you need to return the flow cell to storage.

5. Hold the flow cell by the gray tab with the label facing up.

6. Push to insert flow cell into the slot into the front of the cartridge.

7. An audible click indicates that the flow cell is in place. When properly loaded, the gray tab protrudes from the cartridge.

8. Pull back and remove the gray tab to expose the flow cell.
a. Do not wipe the flow cell with lens paper.

9. Using a new P1000 pipette tip, pierce the library reservoir and push the foil to the edges to enlarge the hole.

10. Discard the pipette tip.

11. Add 20 μL of diluted library to the BOTTOM of the reservoir, avoid touching the foil.

### Initiating a Cloud Run

1. “Start.”

2. Log with basespace account

3. Select the planned run that you previously set up in BaseSpace.

4. Check run information on the screen.

5. Press sequence.

6. Machine will initiate instrument checks. Instrument checks will take a long time.
a. Dry checks warning—the machine will show a prompt that the output folder cannot be accessed. Press ok.

7. Fluidics checks will start, this also takes a long time.

8. The run will start automatically but do not leave until the estimated completion time is shown on the screen.

The examples and embodiments described herein are for illustrative purposes only and various modifications or changes suggested to persons skilled in the art are to be included within the spirit and purview of this application and scope of the appended claims.

1. A method for reconstructing a genome of a pathogen, comprising:
determining an amplification cycle threshold (Ct) value for a plurality of ribonucleic acid (RNA) molecules isolated from a biological sample;
transporting the plurality of RNA molecules at a temperature no greater than −20° C. from a first location to a second location;
performing a reverse transcription polymerase chain reaction (RT-PCR) protocol on at least a portion of the plurality of RNA molecules to generate a plurality of complementary deoxyribonucleic acid (cDNA) molecules; and
sequencing all or a portion of the plurality of cDNA molecules at the second location to determine nucleotide sequences of the all or the portion of the plurality of cDNA molecules.

2. The method of claim 1, wherein the plurality of cDNA molecules comprises a modified nucleic acid.

3. The method of claim 2, wherein the sequencing comprises sequencing the modified nucleic acid at the second location to determine the nucleotide sequences.

4. The method of claim 1, wherein performing the RT-PCR protocol comprises a one-step RT-PCR protocol.

5. The method of claim 4, wherein an input volume of RNA for the one-step RT-PCR protocol is at least about 5 microliters.

6. The method of claim 4, wherein an elongation temperature of the one-step RT-PCR protocol is about 60.5° C.

7. The method of claim 4, wherein an elongation time of the one-step RT-PCR protocol is about 3 minutes.

8. The method of claim 4, wherein performing the one-step RT-PCR protocol comprises use of an RMv1 or RMv2 primer set.

9. The method of claim 4, wherein performing the one-step RT-PCR protocol comprises use of a primer, the primer comprising a 5′ end modification.

10. The method of claim 9, wherein the primer is biotinylated at the 5′ end.

11. The method of claim 1, further comprising tagmenting at least a portion of the plurality of cDNA molecules.

12. The method of claim 11, wherein each cDNA molecule of the plurality of cDNA molecules is tagmented with polyethylene glycol (PEG).

13. The method of claim 11, further comprising performing hybrid capture on at least a portion of the tagmented cDNA molecules.

14. The method of claim 11, further comprising performing hybrid capture on at least a portion of the tagmented cDNA molecules derived from RNA molecules with a Ct value greater than about 27, as determined using a real-time quantitative polymerase chain reaction (RT-qPCR) assay.

15. The method of claim 1, wherein performing the RT-PCR protocol comprises use of an exonuclease with a processivity of at least about 60 nucleotides per second.

16. The method of claim 1, wherein performing the RT-PCR protocol comprises use of Taq polymerase, a tiling primer that is longer than an A400 primer, an A1200 primer, or combinations thereof.

17. (canceled)

18. (canceled)

19. The method of claim 1, wherein the biological sample comprises a saliva sample, a blood sample, a urine sample, a cell lysate, or a tissue biopsy sample, and wherein the biological sample is obtained or derived from a subject or a patient.

20. (canceled)

21. The method of claim 1, further comprising storing data generated by the method in a database accessible through a communication medium, wherein the communication medium comprises a network connection, a wireless connection, an intranet connection, or an internet connection.

22. The method of claim 21, wherein the data comprises the Ct values for the plurality of RNA molecules isolated from the biological sample or the nucleotide sequences.

23. (canceled)

24. A system for high-throughput nucleic acid sequencing and analysis, the system comprising:
a first analysis module configured to determine a plurality of respective amplification cycle threshold (Ct) values for each of a first plurality of nucleic acid populations;
a second analysis module configured to perform a one-step amplification protocol on a subset of the first plurality of nucleic acid populations;
a third analysis module comprising a molecular sequencer configured to determine a first plurality of nucleic acid sequences corresponding to each population of the subset of the first plurality of nucleic acid populations; and
a computer system configured to determine a rate of mutation in a pathogen based at least in part on the first plurality of nucleic acid sequences.

25.-94. (canceled)
